(19) United States
(12) Patent Application Publication
(10) Pub. No.: US 2024/0156954 A1
(43) Pub. Date: May 16, 2024

(54) VACCINE COMPOSITIONS AND METHODS FOR TREATING HSV

(71) Applicants: Redbiotec AG, Schlieren (CH); Eurocine Vaccines AB, Solna (SE)

(72) Inventors: Marina TAMBASCO STUDART, Schlieren (CH); Christian SCHAUB, Schlieren (CH); Corinne JOHN, Schlieren (CH); Martin BÜHLMANN, Schlieren (CH); Martyna WROBLEWSKA, Schlieren (CH); David WILSON, Schlieren (CH); Karl LJUNGBERG, Solna (SE); Hans ARWIDSSON, Solna (SE)

(21) Appl. No.: 18/515,010

(22) Filed: Nov. 20, 2023

Related U.S. Application Data
(63) Continuation-in-part of application No. 18/280,908, filed on Sep. 7, 2023.

(30) Foreign Application Priority Data
Mar. 11, 2021 (EP) 21162170.1

(51) Int. Cl. A61K 39/245; A61K 39/00; A61P 31/22; A61P 37/04

(52) U.S. Cl. CPC A61K 39/245; A61P 31/22; A61P 37/04; A61K 2039/53; A61K 2039/54; A61K 2039/55555

(57) ABSTRACT

The present invention relates to a vaccine composition comprising one or more mRNAs encoding a Herpes Simplex Virus (HSV) structural protein or an immunogenic fragment thereof for the treatment or vaccination against HSV.

### PRIORITY CLAIM

This application is a continuation-in-part of U.S. patent application Ser. No. 18/280,908, filed Sep. 7, 2023, which claims priority to International Application No. PCT/EP2022/056345, filed Mar. 11, 2022, which in turn claims priority to European Patent Application No. 21162170.1, filed Mar. 11, 2021, wherein the entire contents of said applications are incorporated herein by reference in their entireties.

### REFERENCE TO SEQUENCE LISTING

This application contains a Sequence Listing in computer readable form, submitted via USPTO Patent Center. The entire contents of the .xml file entitled “AWA0151US2_Sequence_Listing.xml” created on Nov. 20, 2023, and having a size of 154,693 bytes, is incorporated herein by reference.

### FIELD OF INVENTION

The present invention relates to a vaccine composition comprising one or more mRNAs encoding Herpes Simplex Virus (HSV) structural proteins or an immunogenic fragment thereof for the treatment of or vaccination against HSV.

### BACKGROUND

Herpes simplex virus is a viral genus of the viral family known as Herpesviridae. The species that infect humans are commonly known as Herpes simplex virus 1 (HSV-1) and Herpes simplex virus 2 (HSV-2), wherein their formal names are Human herpesvirus 1 (HHV-1) and Human herpesvirus 2 (HHV-2), respectively. The initial infection with HSV-1 typically occurs during childhood or adolescence and persists lifelong. Infection rates with HSV-1 are between 40% and 80% worldwide, being higher among people of lower socialeconomic status. In many cases people exposed to HSV-1 demonstrate asymptomatic seroconversion. However, initial infection can also be severe, causing widespread 1 to 2 mm blisters associated with severe discomfort that interferes with eating and drinking to the point of dehydration, last 10 to 14 days, and occur 1 to 26 days after inoculation. Recurrent labial herpes affects roughly one third of the US population, and these patients typically experience 1 to 6 episodes per year. Papules on an erythematous base become vesicles within hours and subsequently progress through ulcerated, crusted, and healing stages within 72 to 96 hours (Cernik et al., 2008, Arch Intern Med., vol. 168, pp. 1137-1144). Global estimates in 2003 assume that 16.2% of the population are infected with HSV-2, being the major cause of genital herpes. The ability of the virus to successfully avoid clearance by the immune system by entering a non-replicating state known as latency leads to lifelong infection. Periodic reactivation from latency is possible and leads to viral shedding from the site of the initial infection. Genital lesions due to herpes are often very painful, and can lead to substantial psychological morbidity. The virus can also be passed from mother to child during birth. Without treatment, 80% of infants with disseminated disease die, and those who do survive are often brain damaged. In addition, genital herpes is associated with an increased risk of HIV acquisition by two- to threefold, HIV transmission on a per-sexual act basis by up to fivefold, and may account for 40-60% of new HIV infections in high HSV-2 prevalence populations (Looker et al., 2008, Bulletin of the World Health Organization, vol. 86, pp. 805-812).

Currently, acyclovir, a synthetic acyclic purine-nucleoside analogue, is the standard therapy for HSV infections and has greatly helped control symptoms. Precursor drugs, valacyclovir (converted to acyclovir) and famciclovir (converted to penciclovir), have been licensed and have better oral bioavailability than acyclovir and penciclovir, respectively. The available drugs have an excellent margin of safety because they are converted by viral thymidine kinase to the active drug only inside virally infected cells. However, HSV can develop resistance to acyclovir through mutations in the viral gene that encodes thymidine kinase by generation of thymidine-kinase-deficient mutants or by selection of mutants with a thymidine kinase unable to phosphorylate acyclovir. Most clinical HSV isolates resistant to acyclovir are deficient in thymidine kinase, although altered DNA polymerase has been detected in some. As HSV can lie latent in neurons for months or years before becoming active, such a therapy may be used to treat symptoms caused by HSV but cannot avoid the periodic reactivation of the virus. Accordingly, the most effective and economical way to fight HSV would be a vaccine preventing initial infection and/or periodic reactivation of the virus. A lot of effort has been put in the development of such a vaccine in the past several decades. However, so far attempts to develop a potent HSV vaccine have focused on a limited number of antigens that have shown poor performance in clinical trials. Accordingly, there is an urgent need for a vaccine against HSV. Recent attempts have been made to develop an HSV vaccine based on nucleoside modified mRNAs of HSV glycoproteins (US2020/0276300), however these are still in an early stage of development. There remains a need for further HSV vaccinations.

### SUMMARY OF THE INVENTION

It is an object of the present disclosure to provide immunogenic compositions capable of eliciting an HSV-2-specific immune response in a subject when administered to said subject, which overcome, or at least partially overcome, the disadvantages of the prior art, for example in terms of the elicited immune response.

It is an object of the present disclosure to provide effective HSV-2 vaccine compositions, which overcome, or at least partially overcome, the disadvantages of the prior art, for example in terms of elicited protection.

It is an object of the present disclosure to provide related immunogenic compositions and/or vaccine compositions for use as medicament.

It is an object of the present disclosure to provide medical uses of the immunogenic compositions and/or vaccine compositions for the therapeutic treatment and/or prophylactic treatment of an HSV-1 or HSV-2 infection, such as an HSV-2 infection, such as for the prophylactic treatment of an HSV-1 or HSV-2 infection, such as an HSV-2 infection. Also provided are related treatment methods.

One or more of these objects, and other objects that are apparent to the skilled person from reading the entire disclosure, are met by the various aspects disclosed.

The present inventors have identified a subset of a nucleic acids encoding HSV-2 tegument proteins which are particularly useful in the context of vaccines against an HSV-2 infection. As explained below and as shown in the appended Examples, the inventors have surprisingly found that an immune response elicited by the subset of nucleic acids is particularly beneficial in comparison to an immune response elicited by the tegument proteins encoded by said nucleic acids. Preferably, each of the nucleic acids is capable of eliciting an immune response when administered to a subject.

The term “immunogenic”, as used herein, refers to the properties of an immunogen, which is an entity capable of eliciting humoral and/or cell-mediated immune response. As used herein, the term “fragment” of a protein, such as an immunogenic fragment, is meant to refer to a portion of the amino acid sequence of the full-length polypeptide. In the context of the present disclosure, “an immunogenic fragment” as defined in any one of the herein discussed embodiments, refers to a fragment of an immunogenic protein which retains the same or a similar degree of immunogenicity as the immunogenicity of said protein.

The present invention addresses this need and provides novel immunogenic compositions and vaccine compositions comprising one or more mRNAs, wherein each of said mRNAs encodes a Herpes Simplex Virus (HSV) structural protein or an immunogenic fragment thereof selected from the group consisting of UL48; UL48 and UL49; UL11, UL16 and UL21; or UL31 and UL34. Specifically, in the vaccine composition of the invention, the mRNA encodes UL48 having an amino acid sequence which is 80% or more identical to the amino acid sequence of SEQ ID NO:6, UL49 having an amino acid sequence which is 62% or more identical to the amino acid sequence of SEQ ID NO:7, UL11 having an amino acid sequence which is 75% or more identical to the amino acid sequence of SEQ ID NO:1, UL16 having an amino acid sequence which is 72% or more identical to the amino acid sequence of SEQ ID NO:2, UL21 having an amino acid sequence which is 80% or more identical to the amino acid sequence of SEQ ID NO:3, UL31 having an amino acid sequence which is 85% or more identical to the amino acid sequence of SEQ ID NO:8, and UL34 having an amino acid sequence which is 70% or more identical to the amino acid sequence of SEQ ID NO:8.

In particular, the present invention relates to an immunogenic composition capable of eliciting an HSV-2-specific immune response in a subject when administered to said subject, wherein said immunogenic composition comprises at least one, such as at least two, such as all three nucleic acid(s) selected from the group consisting of:
(i) a nucleic acid encoding a UL11 protein of HSV-2 or an immunogenic fragment thereof, (ii) a nucleic acid encoding a UL16 protein of HSV-2 or an immunogenic fragment thereof, and (iii) a nucleic acid encoding a UL21 protein of HSV-2 or an immunogenic fragment thereof, wherein said UL11 protein comprises an amino acid sequence having at least 80% identity to SEQ ID NO:1, said UL16 protein comprises an amino acid sequence having at least 80% identity to SEQ ID NO:2, and said UL21 protein comprises an amino acid sequence having at least 80% identity to SEQ ID NO:3. In particular, provide are immunogenic composition comprising all three nucleic acids are defined in i), ii) and iii).

Vaccine compositions comprising said immunogenic composition and a pharmaceutically acceptable carrier or excipient are also provided herein.

Preferably, each of the HSV mRNAs in the vaccine composition of the invention is capable of eliciting an immune response when administered in the form of a vaccine composition to a subject.

In addition, the vaccine composition of the invention may further comprise one or more mRNAs encoding a Herpes Simplex Virus (HSV) glycoprotein selected from the group consisting of a) an HSV glycoprotein D (gD) or an immunogenic fragment thereof having an amino acid sequence which is 70% or more identical to the amino acid sequence of SEQ ID NO:11, b) an HSV glycoprotein B (gB) or an immunogenic fragment thereof having an amino acid sequence which is 70% or more identical to the amino acid sequence of SEQ ID NO:10, and c) an HSV glycoprotein E (gE) or an immunogenic fragment thereof having an amino acid sequence which is 70% or more identical to the amino acid sequence of SEQ ID NO:4 or 80% or more identical to the amino acid sequence of SEQ ID NO:5, or any combination thereof.

Specific preferred vaccine compositions comprise structural protein UL48 together with glycoproteins gD and/or gB; structural proteins UL 48 and UL49 together with glycoprotein gE, structural proteins UL11, UL16, and UL21 together with glycoproteins gE, gD, and/or gB, and structural proteins UL31 and UL34 together with glycoproteins gD and/or gB.

The vaccine compositions of the invention can optionally comprise mRNAs encoding Herpes Simplex Virus (HSV) glycoproteins that are nucleoside modified mRNAs comprising one or more pseudouridine residues, preferably where the one or more pseudouridine residues comprise m1ψ (1-methylpseudouridine); m1acp3ψ (1-methyl-3-(3-amino-5-carboxypropyl)pseudouridine, Wm (2′-0-methylpseudouridine), m5D (5-methyldihydrouridine), m3ψ (3-methylpseudouridine), or any combination thereof.

In these specific embodiments of the vaccine composition, the nucleoside modified mRNAs encoding said immunogenic fragments of glycoproteins are selected from the group consisting of:
(1) HSV gD comprises amino acids 26-331 from HSV-2 strain 333, or a homologous sequence from another HSV strain, preferably wherein the nucleic acid sequence of said nucleoside modified mRNA is as set forth in SEQ ID NO:12; and
(2 HSV gE comprises amino acids 24-405 from HSV-2 strain 2.12, or a homologous sequence from another HSV strain, preferably wherein the nucleic acid sequence of said nucleoside modified mRNA is as set forth in SEQ ID NO:13.

Further, the mRNAs in the vaccine compositions of the invention may encode HSV 1 polypeptides, HSV-2 polypeptides or a mixture thereof.

In addition, each of the mRNAs in the vaccine composition may further comprise a poly-A tail, an m7GpppG cap, 3′-0-methyl-m7GpppG cap, or anti-reverse cap analog, a cap-independent translational enhancer, and/or 5′ and 3′ untranslated regions that enhance translation and/or be codon-optimized (e.g., SEQ ID NOs: 25-30).

Furthermore, in the vaccine compositions of the invention, the mRNAs may be encapsulated in a nanoparticle, lipid, polymer, cholesterol, or cell penetrating peptide, preferably in a liposomal nanoparticle.

The vaccine compositions of the invention may be used in treating or preventing a Herpes Simplex Virus (HSV) infection in a subject. Said HSV infection may be selected from the group consisting of an HSV-1 infection, an HSV-2 infection, a primary HSV infection, a flare, recurrence, or HSV labialis following a primary HSV infection, a reactivation of a latent HSV infection, an HSV encephalitis, an HSV neonatal infection, a genital HSV infection, or an oral HSV infection. In particular it is envisioned that the immunogenic composition comprising nucleic acid(s) as defined in i), ii) and iii) and related vaccine compositions are particularly useful for treatment, including therapeutic treatment and/or preventive treatment of disease/initial infection, as well as provide improved immunological control of HSV-2 infection in already infected patients. Without being bound by theory, it is envisioned that this effect is associated with the surprisingly strong induction of antigen-specific T cells, including high secretion of IFN-γ and large numbers of polyfunctional T cells, observed upon administration of the immunogenic composition comprising nucleic acid(s) as defined in i), ii) and iii) as shown in the appended Examples.

The vaccine composition of the invention may be formulated for intramuscular administration, subcutaneous administration, intradermal administration, intranasal, intravaginal, intrarectal administration, or topical administration, preferably wherein the composition is a vaccine for injection, optionally comprising a pharmaceutically acceptable carrier or adjuvant for injection. The vaccine composition of the invention may be used as a medicament and/or for therapy. The vaccine composition of the invention may be used in a method for treating and/or preventing a Herpes Simplex Virus (HSV) infection.

For clarity, in the present disclosure the terms UL11 and RBT26.1 are used interchangeably; the terms UL16 and RBT26.2 are used interchangeably; the terms UL21 and RBT26.3 are used interchangeably. The term RBT26 refers to a composition comprising all three of RBT26.1, RBT26.2 and RBT26.3. Thus, an mRNA RBT26 composition, such as a mRNA RBT26 vaccine composition, comprises RBT26.1 mRNA, RBT26.2 mRNA and RBT26.3 mRNA and a protein RBT26 composition, such as a protein RBT26 vaccine composition, comprises RBT26.1 protein, RBT26.2 protein and RBT26.3 protein.

### OVERVIEW OF THE SEQUENCE LISTING

SEQ ID NO:1 is an exemplary amino acid sequence of UL11 protein of HSV-2.

SEQ ID NO:2 is an exemplary amino acid sequence of UL16 protein of HSV-2.

SEQ ID NO:3 is an exemplary amino acid sequence of UL21 protein of HSV-2.

SEQ ID NO:4 is an exemplary amino acid sequence of gE protein of HSV-2.

SEQ ID NO:5 is an exemplary amino acid sequence of cytoplasmic tail of gE protein of HSV-2.

SEQ ID NO:6 is an exemplary amino acid sequence of UL48 protein of HSV-2.

SEQ ID NO:7 is an exemplary amino acid sequence of UL49 protein of HSV-2.

SEQ ID NO:8 is an exemplary amino acid sequence of UL31 protein of HSV-2.

SEQ ID NO:9 is an exemplary amino acid sequence of UL34 protein of HSV-2.

SEQ ID NO:10 is an exemplary amino acid sequence of gB protein of HSV-2.

SEQ ID NO:11 is an exemplary amino acid sequence of gD protein of HSV-2.

SEQ ID NO:12 is an exemplary gD RNA nucleotide sequence fragment of HSV-2 nucleoside modified (all uridine residues are 1-methyl-pseudouridine).

SEQ ID NO:13 is an exemplary gE RNA nucleotide sequence fragment of HSV-2 nucleoside modified (all uridine residues are 1-methyl-pseudouridine).

SEQ ID NO:14 is an exemplary UL48 of HSV-2 RNA sequence.

SEQ ID NO:15 is an exemplary UL49 of HSV-2 RNA sequence.

SEQ ID NO:16 is an exemplary UL11 of HSV-2 RNA sequence.

SEQ ID NO:17 is an exemplary UL16 of HSV-2 RNA sequence.

SEQ ID NO:18 is an exemplary UL21 of HSV-2 RNA sequence.

SEQ ID NO:19 is an exemplary UL31 of HSV-2 RNA sequence.

SEQ ID NO:20 is an exemplary UL34 of HSV-2 RNA sequence.

SEQ ID NO:21 is an exemplary cytoplasmic tail of gE protein of HSV-2 RNA sequence.

SEQ ID NO:22 is an exemplary gD of HSV-2 RNA sequence.

SEQ ID NO:23 is an exemplary gB of HSV-2 RNA sequence.

SEQ ID NO:24 is an exemplary gE of HSV-2 RNA sequence.

SEQ ID NO:25 is an exemplary codon-optimized UL48 of HSV-2 RNA sequence including exemplary UTRs and exemplary polyA tail, all uridine residues are 1-methyl-pseudouridine. SEQ ID NO:26 is an exemplary codon-optimized UL11 of HSV-2 RNA sequence including exemplary UTRs and exemplary polyA tail, all uridine residues are 1-methyl-pseudouridine.

SEQ ID NO:27 is an exemplary modified (1-methyl-pseudouridine) codon-optimized UL16 of HSV-2 RNA sequence including exemplary UTRs and exemplary polyA tail, all uridine residues are 1-methyl-pseudouridine.

SEQ ID NO:28 is an exemplary modified (1-methyl-pseudouridine) codon-optimized UL21 of HSV-2 RNA sequence including exemplary UTRs and exemplary polyA tail, all uridine residues are 1-methyl-pseudouridine.

SEQ ID NO:29 is an exemplary modified (1-methyl-pseudouridine) codon-optimized gD of HSV-2 RNA sequence including exemplary UTRs and exemplary polyA tail, all uridine residues are 1-methyl-pseudouridine.

SEQ ID NO:30 is an exemplary modified (1-methyl-pseudouridine) codon-optimized ICP4 of HSV-2 RNA sequence including exemplary UTRs and exemplary polyA tail, all uridine residues are 1-methyl-pseudouridine.

SEQ ID NO:31 is an exemplary amino acid sequence of ICP4 protein of HSV-2 (GenBank Accession Number QIH12398.1).

SEQ ID NO:32-34 are exemplary codon-optimized sequences of SEQ ID NO:16-18, respectively.

SEQ ID NO:35-51 are predicted MHC class I and II binding peptides derived from UL11, UL16 and UL21 of HSV-2 as used in the present Examples.

SEQ ID NO:52-54 and 56-57 disclose sequences for CpG oligonucleotide and primers as described in the appended Examples.

SEQ ID NO:55 and 85 refer to the probe with reporter dye used in the Examples, wherein the reporter dye is defined by a DNA sequence region according to SEQ ID NO:55 and an amino acid sequence region according to SEQ ID NO:85.

SEQ ID NO:58-60 are exemplary polyA and 5′UTR and 3′UTR sequences.

SEQ ID NO: 61, 62 and 63 are exemplary codon-optimized sequences of SEQ ID NO:16-18, respectively, each comprising exemplary UTRs and exemplary polyA tail.

SEQ ID NO: 64-66 are codon-optimized gD, gD and gD of HSV-2 RNA sequences including exemplary UTRs and exemplary polyA tail and SEQ ID NO:67-69 are codon-optimized gD, gD and gD of HSV-2 RNA sequences.

SEQ ID NO: 70-72 are exemplary DNA sequence encoding UL11, UL16 and UL21 of HSV-2. SEQ ID NO:73-77 are exemplary DNA sequences encoding the extracellular domain of gE, full length gB, full length gD, full length gE and the cytoplasmic tail of gE of HSV-2, respectively.

SEQ ID NO:78 is an exemplary RNA sequence encoding the cytoplasmic tail of gE of HSV-2.

SEQ ID NO:79 is exemplary codon-optimized RNA sequences encoding the cytoplasmic tail of gE of HSV-2.

SEQ ID NO:80 is exemplary codon-optimized RNA sequences encoding the cytoplasmic tail of gE of HSV-2. comprising exemplary UTRs and exemplary polyA tail.

SEQ ID NO:81 is an exemplary codon-optimized RNA sequence encoding the extracellular domain of gE of HSV-2.

SEQ ID NO:82 is an exemplary codon-optimized RNA sequence encoding the extracellular domain of gE of HSV-2. comprising exemplary UTRs and exemplary polyA tail.

SEQ ID NO:83 is the amino acid sequence of the extracellular domain of gE of HSV-2.

SEQ ID NO:84 is an exemplary RNA sequence encoding the gE of HSV-2.

SEQ ID NO: 86, 87 and 88 are modified (1-methyl-pseudouridine) codon-optimized variants of SEQ ID NO:16, 17 and 18, respectively.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE DRAWINGS

FIG. 1 shows exemplary amino acid sequences with corresponding SEQ ID NOs of the following proteins: UL11 protein of HSV-2, UL16 protein of HSV-2, UL21 protein of HSV-2, gE protein of HSV-2, cytoplasmic tail of gE protein of HSV-2, UL48 protein of HSV-2, UL49 protein of HSV-2, UL31 protein of HSV-2, UL34 protein of HSV-2, gB protein of HSV-2, gD protein of HSV-2.

FIG. 2 shows exemplary nucleotide sequences with corresponding SEQ ID NOs of the following nucleotides: gD RNA nucleotide sequence fragment of HSV-2 nucleoside modified, gE RNA nucleotide sequence fragment of HSV-2 nucleoside modified, UL48 of HSV-2 RNA sequence, UL49 of HSV-2 RNA sequence, UL11 of HSV-2 RNA sequence, UL16 of HSV-2 RNA sequence, UL21 of HSV-2 RNA sequence, UL31 of HSV-2 RNA sequence, UL34 of HSV-2 RNA sequence, Cytoplasmic tail of gE protein of HSV-2 RNA sequence, gD of HSV-2 RNA sequence, gB of HSV-2 RNA sequence, gE of HSV-2 RNA sequence, Codon-optimized UL48 of HSV-2 RNA sequence including UTRs and polyA, Codon-optimized UL11 of HSV-2 RNA sequence including UTRs and polyA, Codon-optimized UL16 of HSV-2 RNA sequence including UTRs and polyA, Codon-optimized UL21 of HSV-2 RNA sequence including UTRs and polyA, Codon-optimized gD of HSV-2 RNA sequence including UTRs and polyA, Codon-optimized ICP4 of HSV-2 RNA sequence including UTRs and polyA.

FIG. 3 shows a Western Blot analysis of an exemplary protein derived from the mRNA UL48 (SEQ ID NO:25). Western blot layout (L-R): Ladder, Positive control, Negative controls (Collected day 2 and 6), Samples from cells transfected with UL48 Pseudouridine (1-methyl-pseudouridine) mRNA (collected days 1, 2, and 6), Samples from cells transfected with UL48 Uridine mRNA (collected days 1, 2, and 6).

FIG. 4 shows a Western Blot analysis of exemplary proteins derived from the mRNAs UL11, UL16 and UL21 (SEQ ID NOs: 26, 27 and 28). Western blot layout (L-R): Positive control, Negative controls (Collected day 2 and 6), Samples from cells transfected with UL11, UL16, and UL21 Pseudouridine (1-methyl-pseudouridine) mRNA (collected days 1 and 2).

FIG. 5 shows a measurement of IFNγ release using ELISA upon incubation with an exemplary UL48 modified mRNA (SEQ ID NO:25). OD450 readings from IFNγ ELISA. Graph layout (Top-Bottom): Blank reading from no-sample ELISA, Negative control sample (non-transfected PBMCs), Samples from PBMCs transfected with UL48 Pseudouridine (1-methyl-pseudouridine) mRNA (collected day 3). Error bars indicate Standard Deviation.

FIG. 6 shows a measurement of IFNγ release using ELISA upon incubation with exemplary ICP4 and gD modified mRNAs (SEQ ID NOs: 30 and 29). OD450 readings from IFNγ ELISA. Graph layout (Top-Bottom): Blank reading from no-sample ELISA, Negative control sample (non-transfected PBMCs), Empty Transfection (PBMCs transfected with no mRNA), Samples from PBMCs transfected with ICP4 Pseudouridine mRNA (collected day 3), Samples from PBMCs transfected with gD Pseudouridine (1-methyl-pseudouridine) mRNA (collected day 3). Error bars indicate Standard Deviation.

FIG. 7 (a) is an image of RBT26.1 (lane 4 and 5), RBT26.2 (lane 8) and RBT26.3 (lane 9) (corresponding to SEQ ID NOs: 26, 27 and 28) mRNA on non-denaturing agarose gel electrophoresis. Lanes 1 and 7 show the ladder (cat no N0362S, New England Biolabs). FIG. 7 (b) shows the results from SDS-PAGE gel electrophoretic analysis. Lane 1: MW marker (Cat no 161-0363, BioRad Switzerland), following lanes are different fractions collected from purification. Lanes 13 and 14 are positive controls from two independent batches. FIG. 7 (c) shows an example of the Western Blot performed using a polyclonal anti-RBT26 serum from previous animal studies to detect protein UL11, UL16 and UL21 (also referred to herein as protein RBT26.1, RBT26.2 and RBT26.3, respectively). Lane 1 is a MW marker (Cat no 161-0385, BioRad, Switzerland) and lanes 2 and 3 show purified protein at two different concentrations.

FIG. 8 (a) shows the immunization schedule and FIG. 8 (b) and (c) show graphs of absolute (b) and relative (c) body weight development during in-life phase of the study of animals administered RBT26 mRNA vaccine, RBT26 protein vaccine and naïve animals as indicated in the Figure. Each dot indicates the geometric mean of the group, and error bars indicate the 95% confidence interval. (n=8 animals per group, n=5 for the naïve control group). One asterisk (*) indicates p<0.05 and **** indicate p<0.0001.

FIG. 9 shows graphs representing serum IgG responses after the prime (a) and boost (b) immunization as assessed by ELISA. The line indicates the geometric mean of each group (n=8 animals per group, n=5 for the naïve control group). Statistical analysis was performed using the non-parametric Kruskal-Wallis followed by Dunn's test for multiple comparisons. One asterisk (*) indicates statistical differences of p<0.05, ** indicate p<0.01, *** indicate p<0.001 and **** indicate p<0.0001.

FIG. 10 shows graphs representing secretion of IFN-γ (a), IL-2 (b), and TNF-α (c) from splenocytes restimulated with the pool of RBT26-derived peptides as determined by Fluorospot analysis from mice immunized with 2, 5 or 15 μg RBT26 mRNA formulated in LNPs or with 1 or 10 g of RBT26 protein together with CpG 1826 and alum, respectively, as well as naïve controls. Statistical analysis was performed using the non-parametric Kruskal-Wallis followed by Dunn's test for multiple comparisons. One asterisk (*) indicates statistical differences of p<0.05, ** indicate p<0.01, *** indicate p<0.001 and **** indicate p<0.0001.

FIG. 11 shows graphs representing secretion of IFN-γ/IL-2 (a), IFN-γ/TNF-α (b), and IL-2/TNF-α (c) from splenocytes restimulated with the pool of RBT26-derived peptides as determined by Fluorospot analysis from mice immunized with 2, 5 or 15 μg RBT26 mRNA formulated in LNPs or with 1 or 10 g of RBT26 protein together with CpG 1826 and alum, respectively, as well as naïve controls. Statistical analysis was performed using the non-parametric Kruskal-Wallis followed by Dunn's test for multiple comparisons. One asterisk (*) indicates statistical differences of p<0.05, ** indicate p<0.01, *** indicate p<0.001 and **** indicate p<0.0001.

FIG. 12 shows graphs representing secretion of IFN-γ/IL-2/TNF-α from splenocytes restimulated with the pool of RBT26-derived peptides as determined by Fluorospot analysis from mice immunized with 2, 5 or 15 μg RBT26 mRNA formulated in LNPs or with 1 or 10 g of RBT26 protein together with CpG 1826 and alum, respectively, as well as naïve controls. Statistical analysis was performed using the non-parametric Kruskal-Wallis followed by Dunn's test for multiple comparisons. One asterisk (*) indicates statistical differences of p<0.05, ** indicate p<0.01, *** indicate p<0.001 and **** indicate p<0.0001.

FIG. 13 is a bargraph representing the total accumulated antigen-specific T cells determined in groups of mice immunized with 2, 5 or 15 μg RBT26 mRNA formulated in LNPs or with 1 or 10 g of RBT26 protein together with CpG 1826 and alum, respectively, as well as naïve controls. Statistical analysis was performed using the non-parametric Kruskal-Wallis followed by Dunn's test for multiple comparisons. One asterisk (*) indicates statistical differences of p<0.05, ** indicate p<0.01, *** indicate p<0.001 and **** indicate p<0.0001.

FIG. 14 shows graphs representing the results obtained from intracellular staining of T cells for IFN-γ and shows the fraction of CD4+(a) and CD8+(b) T cells that produce IFN-γ after restimulation, as determined for groups of mice immunized with 2, 5 or 15 μg RBT26 mRNA formulated in LNPs or with 1 or 10 g of RBT26 protein together with CpG 1826 and alum, respectively, as well as naïve controls. * sign indicates p<0.05, ** indicates a p<0.01, *** indicates a p<0.001 as determined by Kruskal-Wallis analyses followed by Dunn's post hoc analysis for multiple comparisons.

FIG. 15 shows the shows the challenge and two dose administration schedule used in the challenge study described in Example 10.

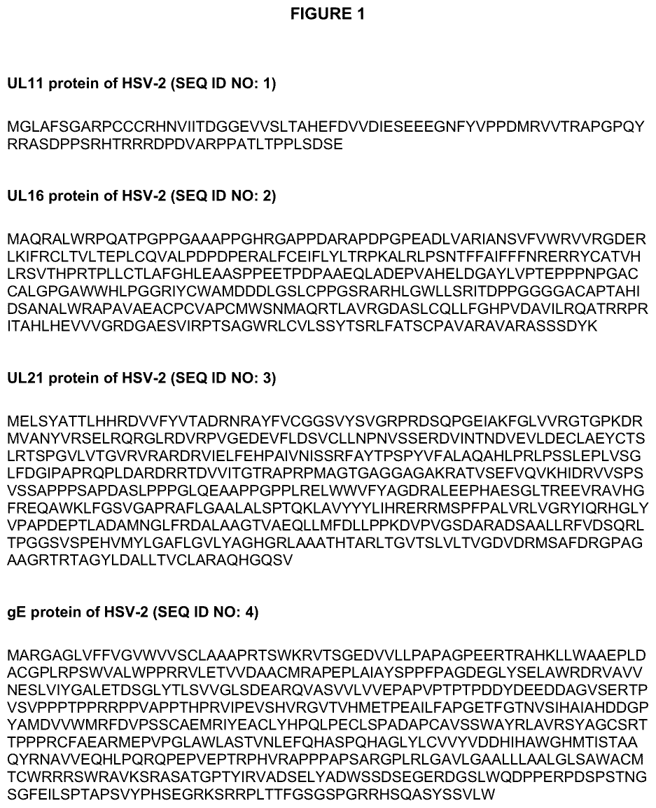

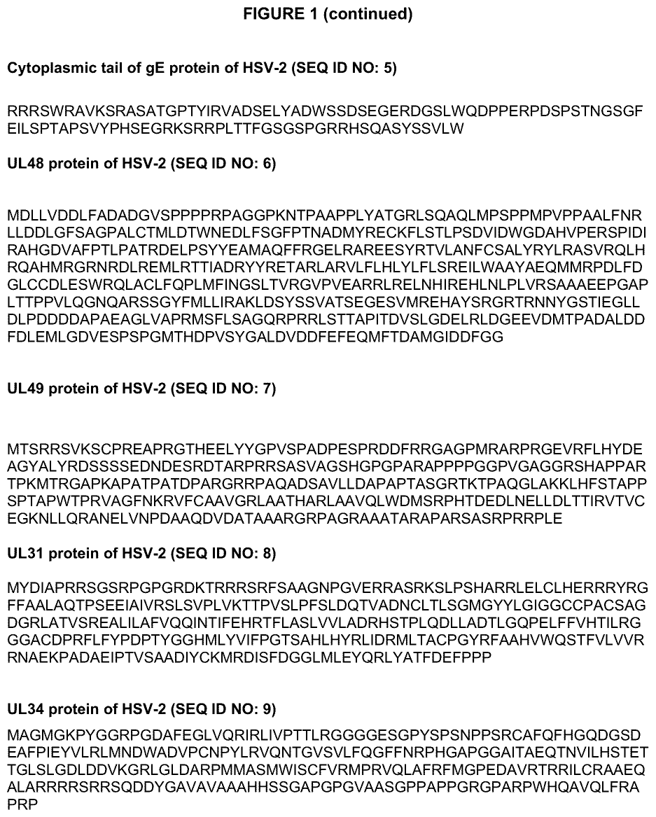

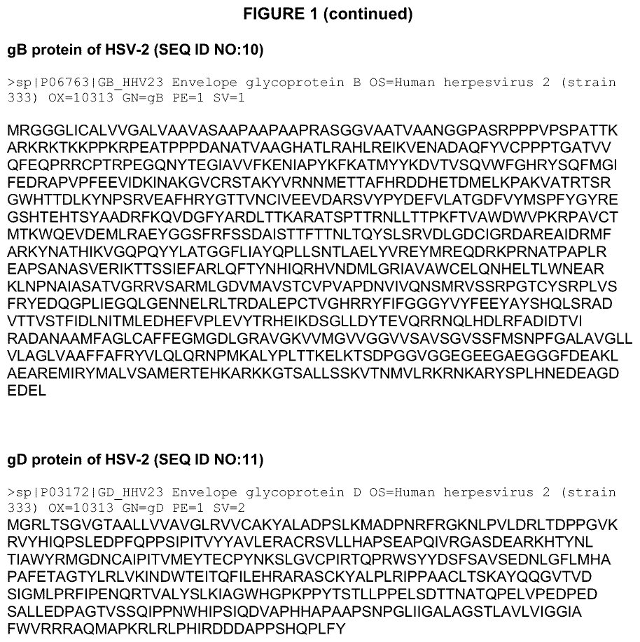

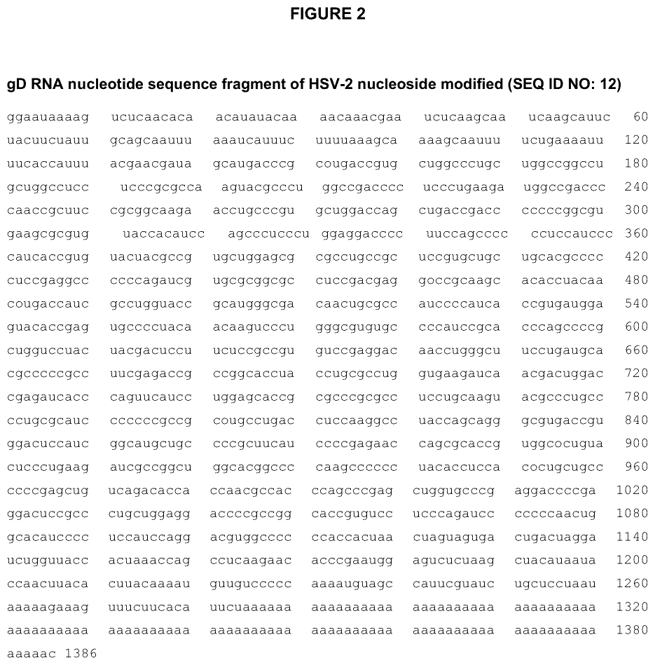

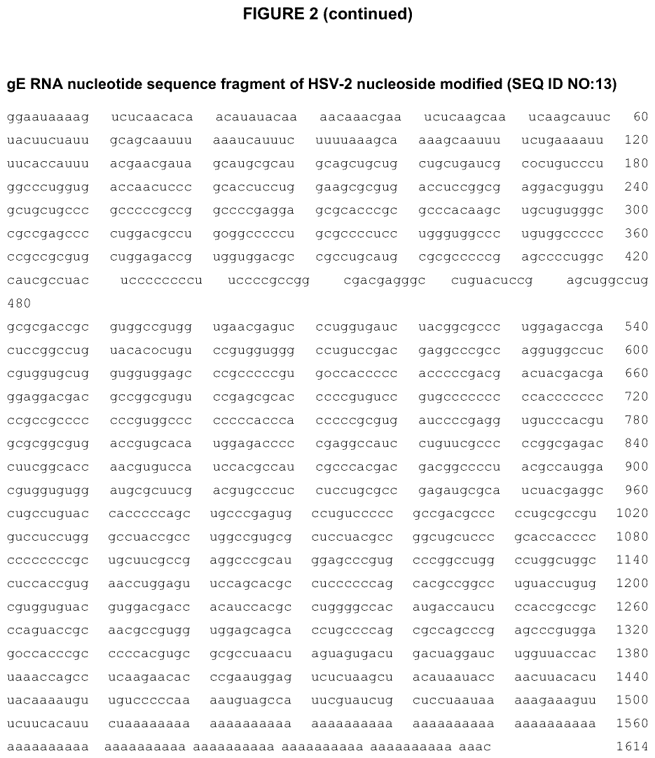

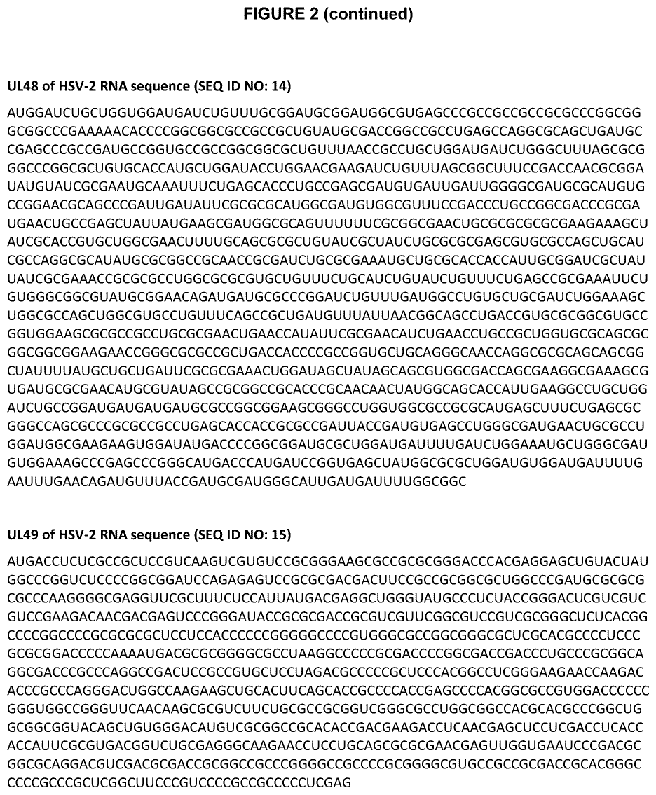

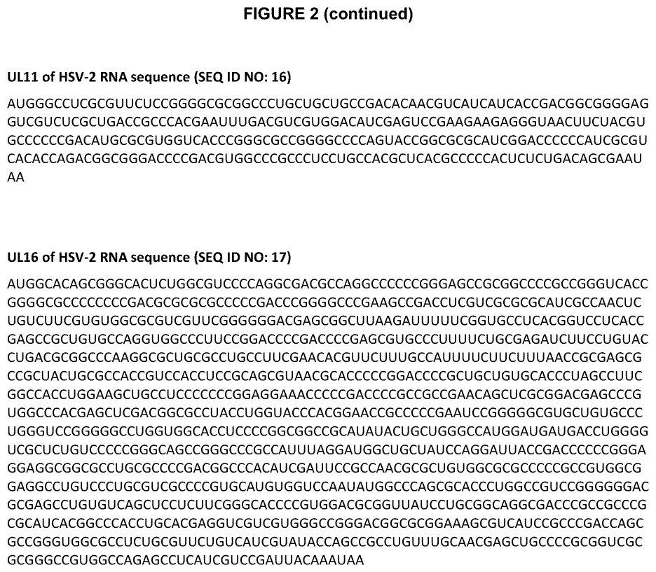

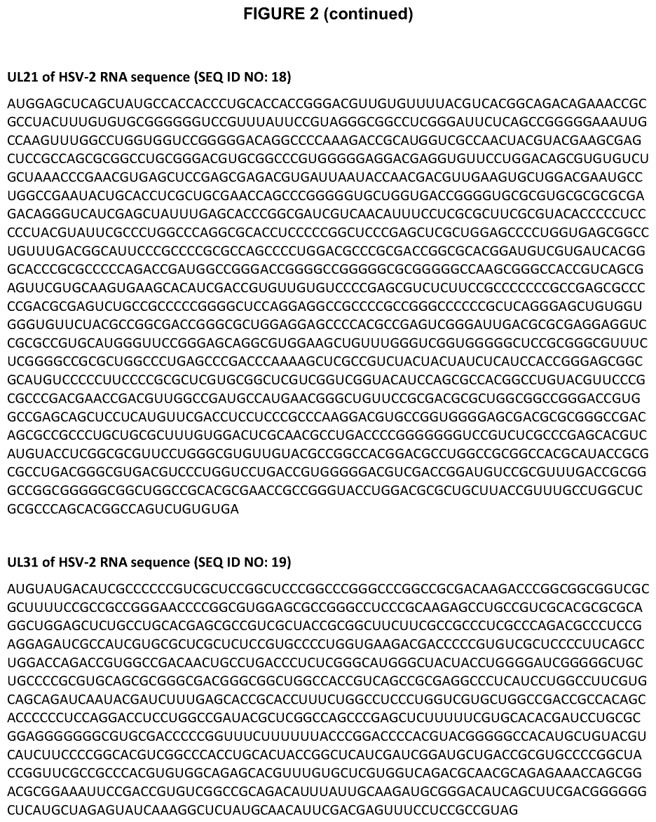

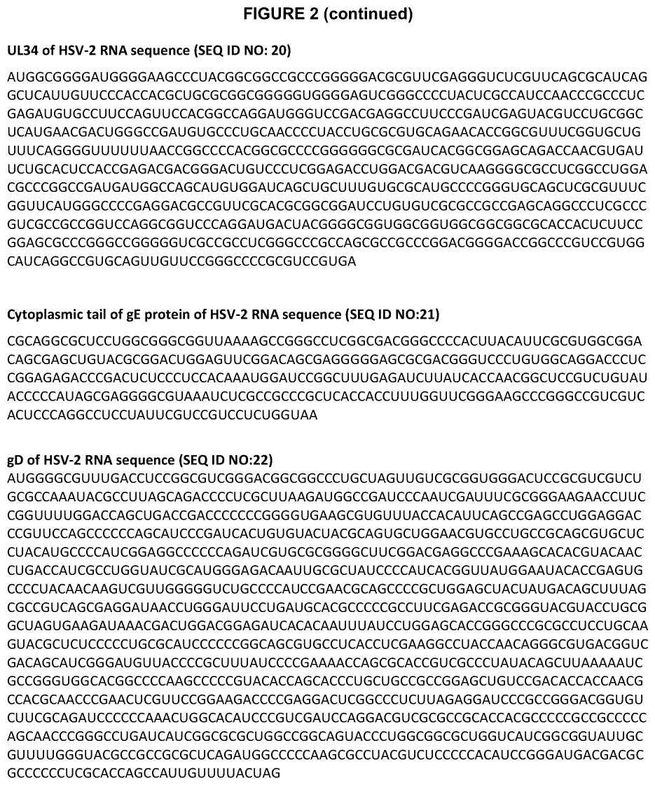

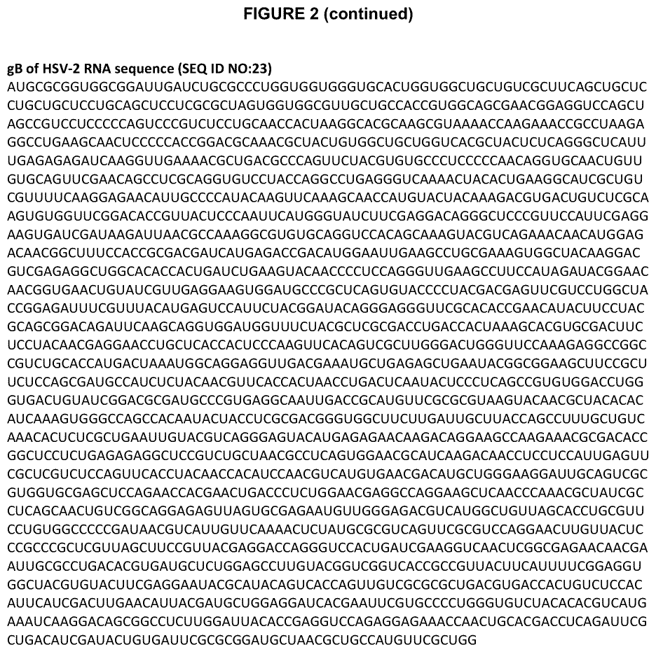

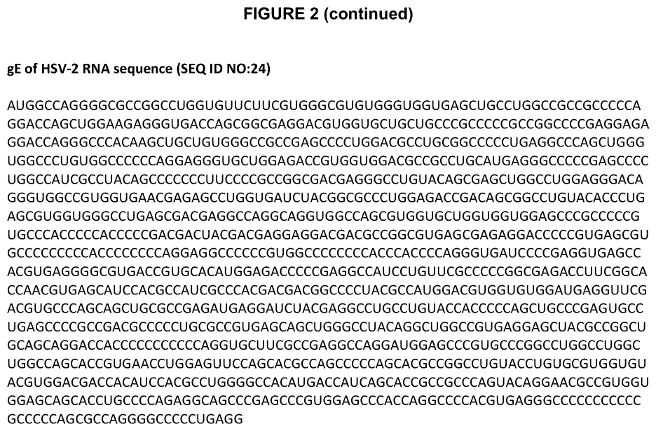

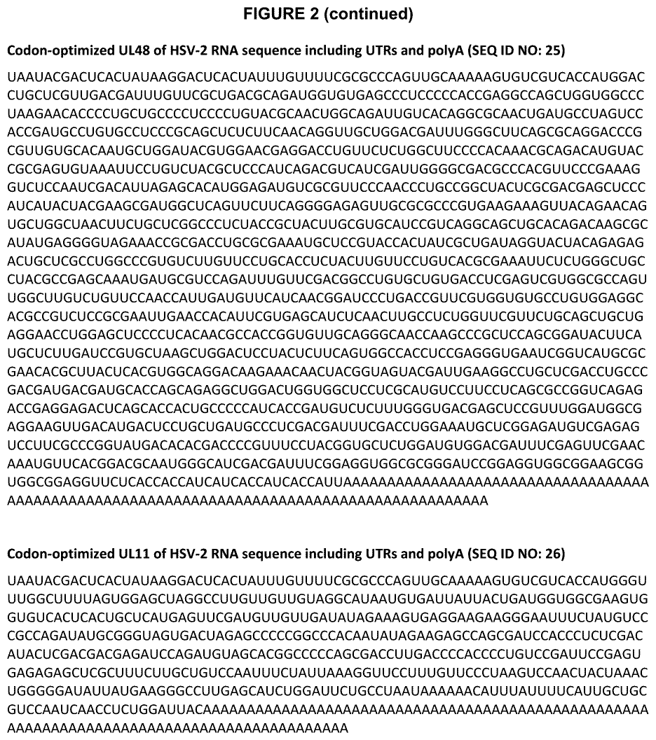

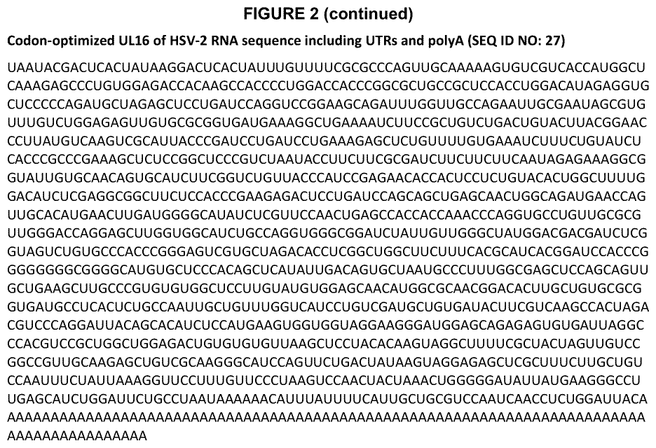

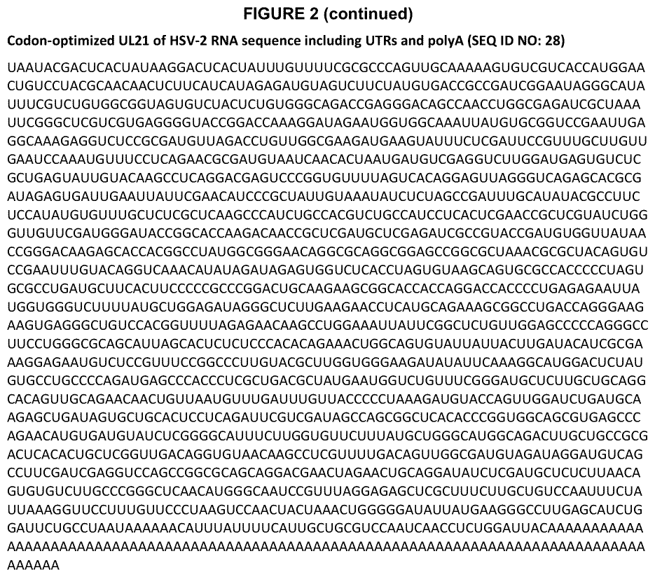

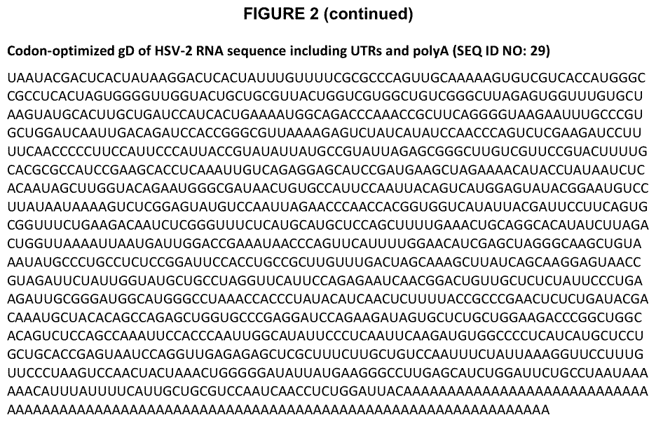

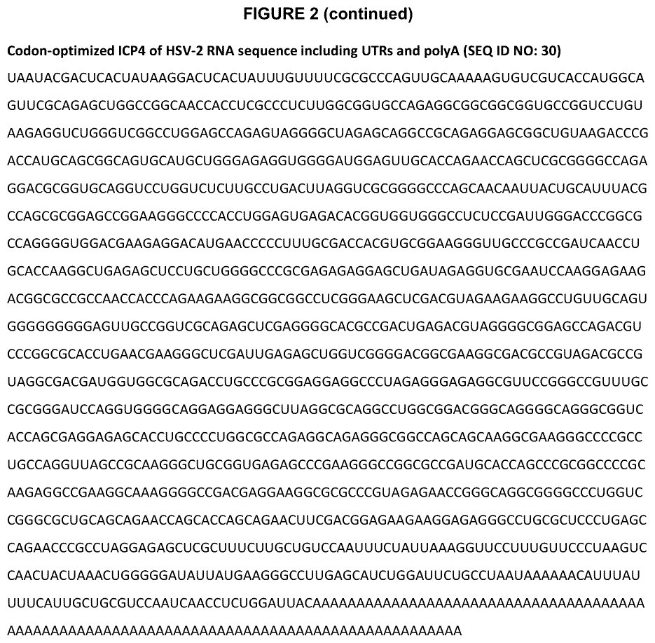

### DETAILED DESCRIPTION

As mentioned above, the present invention provides novel immunogenic compositions and vaccine compositions comprising one or more mRNAs, wherein each of said mRNAs encodes a Herpes Simplex Virus (HSV) structural protein or an immunogenic fragment thereof selected from the group consisting of UL48; UL48 and UL49; UL11, UL16 and UL21; or UL31 and UL34.

While research has focused on using glycoproteins such as gE, gC and gD as antigens (see US2020/0276300, e.g., SEQ ID NOs:4 and 16 therein corresponding to SEQ ID NO:12 and 13 herein), the inventors surprisingly found that immune reactions to mRNA encoding structural HSV proteins are comparably strong. In addition, as structural proteins are generally not glycosylated, it was not necessary to modify the nucleosides in the mRNAs used.

As mentioned above, the present inventors have found that the immunogenic composition which comprises at least one, at least two, and in particular all three nucleic acids as defined in i), ii) and iii) is particularly advantageous in the present context. Thus, in one embodiment, the immunogenic composition comprises all three nucleic acid(s) selected from the group consisting of (i) a nucleic acid encoding a UL11 protein of HSV-2 or an immunogenic fragment thereof, (ii) a nucleic acid encoding a UL16 protein of HSV-2 or an immunogenic fragment thereof, and
(iii) a nucleic acid encoding a UL21 protein of HSV-2 or an immunogenic fragment thereof.

In this context said UL11 protein comprises an amino acid sequence having at least 80% identity to SEQ ID NO:1, said UL16 protein comprises an amino acid sequence having at least 80% identity to SEQ ID NO:2, and said UL21 protein comprises an amino acid sequence having at least 80% identity to SEQ ID NO:3. In particular, provide are immunogenic composition comprising all three nucleic acids are defined in i), ii) and iii). In one embodiment, the immunogenic composition comprises all three nucleic acid(s) selected from the group consisting of (i) a nucleic acid encoding a UL11 protein of HSV-2, (ii) a nucleic acid encoding a UL16 protein of HSV-2, and (iii) a nucleic acid encoding a UL21 protein of HSV-2.

As the skilled person will realize, the properties of a polypeptide, such as the immunogenicity of the polypeptides of the present disclosure, may be dependent on the sequence structure of the polypeptide and the presence and accessibility of immunogenic regions within said polypeptide. It is therefore possible to make minor changes to the sequence of amino acids in a polypeptide without affecting the function thereof. Thus, the disclosure encompasses uses of nucleic acids encoding modified variants of the immunogenic polypeptide as described herein, which are such that the immunogenic characteristics are retained.

For example, it is possible that one or several amino acid residues belonging to a certain functional grouping of amino acid residues (e.g. hydrophobic, hydrophilic, polar etc.) could be exchanged for another amino acid residue from the same functional group. It is also possible, that one or several amino acid residues are exchanged for one or several amino acid residues that belong to a different functional group, provided that the resulting polypeptide retains its immunogenic properties.

Thus, in one embodiment, the UL11 protein of HSV-2 comprises or consists of an amino acid sequence selected from a group consisting of SEQ ID NO:1 and any amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, identity to SEQ ID NO:1. In a particular embodiment, said UL11 protein of HSV-2 comprises or consists of an amino acid sequence according to SEQ ID NO:1.

In one embodiment, the UL16 protein of HSV-2 comprises or consists of an amino acid sequence selected from a group consisting of SEQ ID NO:2 and any amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, identity to SEQ ID NO:2. In a particular embodiment, said UL16 protein of HSV-2 comprises or consists of an amino acid sequence according to SEQ ID NO:2.

In one embodiment, the UL21 protein of HSV-2 comprises or consists of an amino acid sequence selected from a group consisting of SEQ ID NO:3 and any amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, identity to SEQ ID NO:3. In a particular embodiment, said UL21 protein of HSV-2 comprises or consists of an amino acid sequence according to SEQ ID NO:3.

In one embodiment, each nucleic acid as defined in i), ii) and/or iii) is present in an individual nucleic acid construct or wherein at least two of said nucleic acid as defined in i), ii) and/or iii) are present in the same nucleic acid construct, such as wherein all three of said nucleic acid as defined in i), ii) and/or iii) are present in the same nucleic acid construct. In one embodiment, at least two of said nucleic acid as defined in i), ii) and/or iii) are present in the same nucleic acid construct. In one embodiment, all three of said nucleic acid as defined in i), ii) and/or iii) are present in the same nucleic acid construct. In one embodiment, each nucleic acid as defined in i), ii) and/or iii) is present in an individual nucleic acid construct.

In one embodiment, the nucleic acid as defined in i), ii) and/or iii) is selected from the group consisting of DNA and RNA. In one embodiment, the nucleic acid as defined in i), ii) and/or iii) is DNA.

As used herein, the term “DNA” refers to a deoxyribonucleic acid that carries genetic information for one or more proteins in the context of the present disclosure. Generally, such a DNA encodes a polypeptide and is transcribed into a messenger RNA (mRNA) which in turn is translated into the encoded protein in the target cell.

In one embodiment, the nucleic acid as defined in i) comprises a nucleic acid sequence according to SEQ ID NO:69 or any sequence having at least 80%, such as at least 85%, such as at least 90%, such as at least 95%, such as at least 98%, such as at least 99% identity to SEQ ID NO.69. In one embodiment, the nucleic acid as defined in ii) comprises a nucleic acid sequence according to SEQ ID NO:70 or any sequence having at least 80%, such as at least 85%, such as at least 90%, such as at least 95%, such as at least 98%, such as at least 99% identity to SEQ ID NO.70. In one embodiment, the nucleic acid as defined in iii) comprises a nucleic acid sequence according to SEQ ID NO:71 or any sequence having at least 80%, such as at least 85%, such as at least 90%, such as at least 95%, such as at least 98%, such as at least 99% identity to SEQ ID NO.71.

In one embodiment, the nucleic acid as defined in i), ii) and/or iii) is inserted into a plasmid or a vector. In one embodiment, said nucleic acid as defined in i), ii) and/or iii) is inserted into a plasmid. In one embodiment, the nucleic acid as defined in i), ii) and/or iii) is inserted into a vector. Said vector may be a viral vector. Said viral vector may be an adenoviral vector.

In another embodiment, the nucleic acid as defined in i), ii) and/or iii) is RNA. In one particular embodiment, the RNA is messenger RNA (mRNA), such as codon-optimized mRNA. Said RNA may be obtained by an RNA manufacturing method, such as by in vitro transcription.

As used herein the term “mRNA” refers to a messenger ribonucleic acid. Generally, such an mRNA encodes a polypeptide and is translated into the protein it encodes in the target cell. To enhance such translation, the mRNA may further comprise a poly-A tail, an m7GpppG cap, 3′-0-methyl-m7GpppG cap, or anti-reverse cap analog, a cap-independent translational enhancer, and/or 5′ and 3′ untranslated regions that enhance translation (e.g., as shown in SEQ ID NOs: 25-30 herein). As appreciated by those skilled in the art, codon optimality is one feature that contributes greatly to mRNA stability and is this is discussed further below. In one embodiment wherein the nucleic acid is RNA, the nucleic acid as defined in i) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:16 and any codon-optimized version thereof, such as a group consisting of SEQ ID NO:16 and any codon-optimized version thereof. In one embodiment, the nucleic acid as defined in i) comprises a nucleic acid sequence selected from a group consisting of SEQ ID NO:16 and SEQ ID NO:32. In one embodiment, the nucleic acid as defined in i) comprises a nucleic acid sequence selected from any codon-optimized version of SEQ ID NO:16.

In one particular embodiment, the nucleic acid as defined in i) comprises a nucleic acid sequence according to SEQ ID NO:32.

In one embodiment wherein the nucleic acid is RNA, the nucleic acid as defined in ii) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:17 and any codon-optimized version thereof, such as a group consisting of SEQ ID NO:17 and any codon-optimized version thereof. In one embodiment, the nucleic acid as defined in i) comprises a nucleic acid sequence selected from a group consisting of SEQ ID NO:17 and SEQ ID NO:33. In one embodiment, the nucleic acid as defined in ii) comprises a nucleic acid sequence selected from any codon-optimized version of SEQ ID NO:17. In one particular embodiment, the nucleic acid as defined in ii) comprises a nucleic acid sequence according to SEQ ID NO:33.

In one embodiment wherein the nucleic acid is RNA, the nucleic acid as defined in iii) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:18 and any codon-optimized version thereof, such as a group consisting of SEQ ID NO:18 and any codon-optimized version thereof. In one embodiment, the nucleic acid as defined in iii) comprises a nucleic acid sequence selected from a group consisting of SEQ ID NO:18 and SEQ ID NO:34. In one embodiment, the nucleic acid as defined in iii) comprises a nucleic acid sequence selected from any codon-optimized version of SEQ ID NO:18. In one particular embodiment, the nucleic acid as defined in iii) comprises a nucleic acid sequence according to SEQ ID NO:34.

Polyadenylation is the addition of a poly-A tail (i.e. polyadenylated 3′-ends) to an RNA transcript, typically an mRNA. A poly-A tail consists of multiple adenosine monophosphates—in other words, it is a stretch of RNA that has only adenine bases. The poly-A tail is known to be advantageous for the nuclear export, translation and/or stability of mRNA. The tail is shortened over time in the cells and when it is short enough, the mRNA is enzymatically degraded.

Without being bound by theory, such poly-A tail may thus be beneficial in the context of the present disclosure. Accordingly, in one embodiment, the nucleic acid as defined in i), ii) and/or iii) comprise(s) a poly-A tail. In one particular embodiment, said poly-A tail comprises a nucleic acid sequence according to SEQ ID NO:58.

In molecular genetics, an untranslated region (or UTR) refers to either of two sections, one on each side of a coding sequence on a strand of mRNA. If it is found on the 5′ side, it is called the 5′ UTR (also known as leader sequence) or if it is found on the 3′ side, it is called the 3′ UTR (also known as trailer sequence). Within the 5′ UTR, which is thus upstream from the coding sequence, a sequence region is recognized by the ribosome which allows the ribosome to bind and initiate translation. The 3′ UTR, which is thus found immediately following the translation stop codon, plays a critical role in translation termination as well as post-transcriptional modifications. These regions are thus involved in translation regulation and maintaining mRNA stability. Without being bound by theory, the present inventors envision that such 5′ UTR and/or 3′ UTR region(s) may be beneficial in the context of the present disclosure. Thus, in one embodiment, the nucleic acid as defined in i), ii) and/or iii) comprise(s) a 5′ untranslated region, such as wherein said 5′ untranslated region enhances translation. In one embodiment, the 5′ untranslated region comprises a nucleic acid sequence according to SEQ ID NO:59. In another embodiment, the nucleic acid as defined in i), ii) and/or iii) comprise(s) a 3′ untranslated region, such as wherein said 3′ untranslated region enhances translation. In one embodiment, the 3′ untranslated region comprises a nucleic acid sequence according to SEQ ID NO:60. In a particular embodiment, the nucleic acid as defined in i), ii) and/or iii) comprise(s) both a 5′ untranslated region and a 3′ untranslated region. In another particular embodiment, the nucleic acid as defined in i), ii) and/or iii) comprise(s) both a 5′ untranslated region comprising a nucleic acid sequence according to SEQ ID NO:59 and a 3′ untranslated region comprising a nucleic acid sequence according to SEQ ID NO:60.

A “polypeptide” refers to a molecule comprising a polymer of amino acids linked together by peptide bonds. Said term is not meant herein to refer to a specific length of the molecule and is therefore herein interchangeably used with the term “protein”. When used herein, the term “polypeptide” or “protein” also includes a “polypeptide of interest” or “protein of interest” which is expressed by the expression cassettes or vectors or can be isolated from the host cells of the invention. A polypeptide comprises an amino acid sequence, and, thus, sometimes a polypeptide comprising an amino acid sequence is referred to herein as a “polypeptide comprising a polypeptide sequence”. Thus, herein the term “polypeptide sequence” is interchangeably used with the term “amino acid sequence”.

The term “amino acid” or “aa” herein refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

The term “Herpes Simplex Virus” and “HSV” are used interchangeably herein and refer generally to the viruses of the herpesviral Genus Simplexvirus, i.e. Ateline herpesvirus 1, Bovine herpesvirus 2, Cercopithecine herpesvirus 1, Cercopithecine herpesvirus 2, Cercopithecine herpesvirus 16, Human herpesvirus 1, Human herpesvirus 2, Macropodid herpesvirus 1, Macropodid herpesvirus 2, Saimiriine herpesvirus 1. Preferred viral species of the Genus Simplex virus are viruses infecting humans. Even more preferred viral species are Herpes simplex virus 1 (HSV-1) and Herpes simplex virus 2 (HSV-2) which are also known as human herpesvirus 1 and 2 (HHV-1 and HHV-2), respectively. Even more preferred viral species is HSV-2.

In particular, the present inventors have found that the immunogenic composition comprising at least one, such as at least two, such as all three nucleic acid(s) selected from the group consisting of: (i) a nucleic acid encoding a UL11 protein of HSV-2 or an immunogenic fragment thereof, (ii) a nucleic acid encoding a UL16 protein of HSV-2 or an immunogenic fragment thereof, and (iii) a nucleic acid encoding a UL21 protein of HSV-2 or an immunogenic fragment thereof, as defined herein, is particularly useful for the therapeutic treatment and/or prophylactic treatment of an HSV-2 infection as it is capable of eliciting surprisingly beneficial HSV-2-specific immune response in a subject when administered to said subject as shown in the appended Examples.

The term “vaccine composition” as used herein relates to a composition comprising the mRNAs of the present invention which can be used to prevent or treat a pathological condition associated with HSV in a subject. The “vaccine composition” may or may not include one or more additional components that enhance the immunological activity of the active component or such as buffers, reducing agents, stabilizing agents, chelating agents, bulking agents, osmotic balancing agents (tonicity agents); surfactants, polyols, anti-oxidants; lyoprotectants; anti-foaming agents; preservatives; and colorants, detergents, sodium salts, and/or antimicrobials etc. The vaccine composition may additionally comprise further components typical to pharmaceutical compositions. The vaccine of the present invention is, preferably, for human and/or veterinary use. The vaccine composition may be sterile and/or pyrogen-free. The vaccine composition may be isotonic with respect to humans.

The vaccine composition preferably comprises a therapeutically effective amount of the mRNAs of the invention.

It will be appreciated by the skilled person that the present invention differs from naturally occurring phenomena and therefore is part of patent eligible subject matter according to US patent practise. In particular, the present immunogenic composition differs from naturally occurring virus as it does not comprise a viral envelope and/or comprises non-naturally occurring substitution modification or non-naturally occurring nucleoside modification in comparison to the naturally occurring nucleic acids which encode for the UL11, UL16 and U21 proteins of HSV-2. Thus, in one embodiment of the first aspect, there is provided an immunogenic composition as defined herein, wherein the composition does not comprise a viral envelope. In one embodiment, the immunogenic composition as defined herein does not comprise a viral capsid. In other embodiments, the immunogenic composition does not comprise any HSV-2 glycoproteins or does not comprise any nucleic acids encoding such any HSV-2 glycoproteins. In one embodiment, said immunogenic composition does not comprise any gE protein (SEQ ID NO:4) or fragment thereof. In one embodiment, said immunogenic composition does not comprise any nucleic acid encoding gE protein (SEQ ID NO:4) or fragment thereof.

In one embodiment, said immunogenic composition does not comprise any gB protein (SEQ ID NO:10) or fragment thereof. In one embodiment, said immunogenic composition does not comprise any nucleic acid encoding gB protein (SEQ ID NO:10) or fragment thereof. In one embodiment, said immunogenic composition does not comprise any gD protein (SEQ ID NO:11) or fragment thereof. In one embodiment, said immunogenic composition does not comprise any nucleic acid encoding gD protein (SEQ ID NO:11) or fragment thereof.

In one embodiment, said nucleic acid as defined in i), ii) and/or iii) is selected from the group consisting of DNA and RNA, such as wherein said DNA or RNA comprises at least one non-naturally occurring substitution modification or non-naturally occurring nucleoside modification in comparison to the naturally occurring nucleic acids which encode for the UL11, UL16 and U21 proteins of HSV-2, respectively. In one particular embodiment, said nucleic acid as defined in i) is RNA selected from the group consisting of nucleic acid sequenced that have at least 80%, such as at least 85%, such as at least 90%, such as at least 95%, such as at least 98%, such as at least 99% identity to SEQ ID NO:16 and contain at least one non-naturally occurring nucleoside modification relative to SEQ ID NO:16; said nucleic acid as defined in ii) is RNA selected from the group consisting of nucleic acid sequences that have at least 80%, such as at least 85%, such as at least 90%, such as at least 95%, such as at least 98%, such as at least 99% identity to SEQ ID NO:17 and contain at least one non-naturally occurring nucleoside modification relative to SEQ ID NO:17; and/or said nucleic acid as defined in iii) is RNA selected from the group consisting of nucleic acid sequences that have at least 80%, such as at least 85%, such as at least 90%, such as at least 95%, such as at least 98%, such as at least 99% identity to SEQ ID NO:18 and contain at least one non-naturally occurring nucleoside modification relative to SEQ ID NO:18.

In one particular embodiment, said nucleic acid as defined in i) is RNA selected from the group consisting of nucleic acid sequences that have at least 80%, such as at least 85%, such as at least 90%, such as at least 95%, such as at least 98%, such as at least 99% identity to SEQ ID NO:16 and contain at least one non-naturally occurring substitution modification relative to SEQ ID NO:16; said nucleic acid as defined in ii) is RNA selected from the group consisting of nucleic acid sequences that have at least 80%, such as at least 85%, such as at least 90%, such as at least 95%, such as at least 98%, such as at least 99% identity to SEQ ID NO:17 and contain at least one non-naturally occurring substitution modification relative to SEQ ID NO:17; and/or said nucleic acid as defined in iii) is RNA selected from the group consisting of nucleic acid sequences that have at least 80%, such as at least 85%, such as at least 90%, such as at least 95%, such as at least 98%, such as at least 99% identity to SEQ ID NO:18 and contain at least one non-naturally occurring substitution modification relative to SEQ ID NO:18.

As appreciated by those skilled in the art, codon optimality is one feature that contributes greatly to mRNA stability. Stable mRNAs are enriched in codons designated optimal, whereas unstable mRNAs contain predominately non-optimal codons. Substitution of optimal codons with synonymous, non-optimal codons results in dramatic mRNA destabilization, while the converse substitution significantly increases stability. Codon optimality impacts ribosome translocation, connecting the processes of translation elongation and decay through codon optimality (Presnyak et al, Cell, 160(6):1111-24 (2015)). The skilled person is familiar with the concept of codon optimization and knows that it refers to experimental approaches designed to improve the codon composition of a recombinant gene based on various criteria without altering the amino acid sequence encoded by the nucleic acid. In the present context, the term “codon-optimized” refers to nucleic acid sequences comprising modified codons compared to the native naturally occurring sequences. As demonstrated in the appended Examples, codon optimized versions of the herein disclosed mRNAs are useful in the immunogenic composition as disclosed herein. The skilled person is aware of that several different approaches and algorithms for codon optimization are known in the art and appreciates the present disclosure presents non-limiting examples of codon-optimized sequences, such as sequences comprising SEQ ID NO: 32, 33 and 34. In other words, the skilled person appreciates that any codon-optimized sequence of SEQ ID NO:16, 17 and 18 may be used in the context of the present invention.

In one embodiment, there is provided a immunogenic composition as disclosed herein, wherein the nucleic acid as defined in i) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:16 and any codon-optimized version thereof, such as a group consisting of SEQ ID NO:16 and any codon-optimized version thereof, such as wherein the nucleic acid as defined in i) comprises a nucleic acid sequence selected from a group consisting of SEQ ID NO:16 and SEQ ID NO:32. In one embodiment, said nucleic acid as defined in i) comprises a nucleic acid sequence selected from any codon-optimized version of SEQ ID NO:16. In one embodiment, said nucleic acid as defined in i) comprises a nucleic acid sequence selected from a group consisting a sequence having at least 80%, such as at least 85%, such as at least 90%, such as at least 95%, such as at least 98%, such as at least 99% identity with SEQ ID NO:32. In one embodiment, said nucleic acid as defined in i) comprises a nucleic acid sequence according to SEQ ID NO:32.

Thus, in one embodiment, the nucleic acid as defined in i) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:16 and any codon-optimized or nucleoside modified version of SEQ ID NO:16, such as a group consisting of SEQ ID NO:16 and any codon-optimized or nucleoside modified version thereof. In one embodiment, the nucleic acid as defined in i) comprises a nucleic acid sequence selected from a group consisting of SEQ ID NO:16 and any codon-optimized and/or nucleoside modified version thereof, such as wherein the nucleic acid as defined in i) comprises a nucleic acid sequence selected from a group consisting of SEQ ID NO:16 and SEQ ID NO:32. In one embodiment, the nucleic acid as defined in i) comprises a nucleic acid sequence selected from any codon-optimized or nucleoside modified version of SEQ ID NO:16, such as any codon-optimized and nucleoside modified version of SEQ ID NO:16, such as any nucleoside modified version of SEQ ID NO:32. In one embodiment, the nucleic acid as defined in i) comprises a nucleic acid sequence according to SEQ ID NO:32 or SEQ ID NO:86. In one embodiment, the nucleic acid as defined in i) comprises a nucleic acid sequence according SEQ ID NO:86 or any nucleic acid sequence having at least 80%, such as at least 85%, such as at least 90%, such as at least 95%, such as at least 98%, such as at least 99% identity to SEQ ID NO:86.

In one embodiment, there is provided a immunogenic composition as disclosed herein, wherein the nucleic acid as defined in ii) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:17 and any codon-optimized version thereof, such as a group consisting of SEQ ID NO:17 and any codon-optimized version thereof, such as wherein the nucleic acid as defined in ii) comprises a nucleic acid sequence selected from a group consisting of SEQ ID NO:17 and SEQ ID NO:33. In one embodiment, said nucleic acid as defined in ii) comprises a nucleic acid sequence selected from any codon-optimized version of SEQ ID NO:17. In one embodiment, said nucleic acid as defined in ii) comprises a nucleic acid sequence selected from a group consisting a sequence having at least 80%, such as at least 85%, such as at least 90%, such as at least 95%, such as at least 98%, such as at least 99% identity with SEQ ID NO:33. In one embodiment, said nucleic acid as defined in ii) comprises a nucleic acid sequence according to SEQ ID NO:33.

Thus, in one embodiment, the nucleic acid as defined in ii) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:17 and any codon-optimized or nucleoside modified version of SEQ ID NO:17, such as a group consisting of SEQ ID NO:17 and any codon-optimized or nucleoside modified version thereof. In one embodiment, said nucleic acid as defined in ii) comprises a nucleic acid sequence selected from a group consisting of SEQ ID NO:17 and any codon-optimized and/or nucleoside modified version thereof, such as wherein the nucleic acid as defined in ii) comprises a nucleic acid sequence selected from a group consisting of SEQ ID NO:17 and SEQ ID NO:33. In one embodiment, said nucleic acid as defined in ii) comprises a nucleic acid sequence selected from any codon-optimized or nucleoside modified version of SEQ ID NO:17, such as any codon-optimized and nucleoside modified version of SEQ ID NO:17, such as nucleoside modified version of SEQ ID NO:33. In one embodiment, said nucleic acid as defined in ii) comprises a nucleic acid sequence according to SEQ ID NO:33 or SEQ ID NO:87. In one embodiment, the nucleic acid as defined in ii) comprises a nucleic acid sequence according SEQ ID NO:87 or any nucleic acid sequence having at least 80%, such as at least 85%, such as at least 90%, such as at least 95%, such as at least 98%, such as at least 99% identity to SEQ ID NO:87.

In one embodiment, there is provided a immunogenic composition as disclosed herein, wherein the nucleic acid as defined in iii) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:18 and any codon-optimized version thereof, such as a group consisting of SEQ ID NO:18 and any codon-optimized version thereof, such as wherein the nucleic acid as defined in iii) comprises a nucleic acid sequence selected from a group consisting of SEQ ID NO:18 and SEQ ID NO:34. In one embodiment, said nucleic acid as defined in i) comprises a nucleic acid sequence selected from any codon-optimized version of SEQ ID NO:18. In one embodiment, said nucleic acid as defined in iii) comprises a nucleic acid sequence selected from a group consisting a sequence having at least 80%, such as at least 85%, such as at least 90%, such as at least 95%, such as at least 98%, such as at least 99% identity with SEQ ID NO:34. In one embodiment, said nucleic acid as defined in i) comprises a nucleic acid sequence according to SEQ ID NO:34. Thus, in one embodiment, the nucleic acid as defined in iii) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:18 and any codon-optimized or nucleoside modified version of SEQ ID NO:18, such as a group consisting of SEQ ID NO:18 and any codon-optimized or nucleoside modified version thereof. In one embodiment, the nucleic acid as defined in iii) comprises a nucleic acid sequence selected from a group consisting of SEQ ID NO:18 and any codon-optimized and/or nucleoside modified version thereof, such as wherein the nucleic acid as defined in iii) comprises a nucleic acid sequence selected from a group consisting of SEQ ID NO:18 and SEQ ID NO:34. In one embodiment, said nucleic acid as defined in iii) comprises a nucleic acid sequence selected from any codon-optimized or nucleoside modified version of SEQ ID NO:18, such as any codon-optimized and nucleoside modified version of SEQ ID NO:18, such as nucleoside modified version of SEQ ID NO:34. In one embodiment, said nucleic acid as defined in iii) comprises a nucleic acid sequence according to SEQ ID NO:34 or SEQ ID NO.88. In one embodiment, the nucleic acid as defined in iii) comprises a nucleic acid sequence according SEQ ID NO:88 or any nucleic acid sequence having at least 80%, such as at least 85%, such as at least 90%, such as at least 95%, such as at least 98%, such as at least 99% identity to SEQ ID NO:88.

In one particular embodiment of the immunogenic composition as defined herein, the nucleic acid as defined in i) comprises a nucleic acid sequence selected from a group consisting of SEQ ID NO:16 and any codon-optimized version thereof; the nucleic acid as defined in ii) comprises a nucleic acid sequence selected from a group consisting of SEQ ID NO:17 and any codon-optimized version thereof; and wherein the nucleic acid as defined in iii) comprises a nucleic acid sequence selected from a group consisting of SEQ ID NO:18 and any codon-optimized version thereof and optionally wherein the composition does not comprise a viral envelope. In one embodiment, said immunogenic composition comprises a nucleic acid sequence selected from a group consisting of any codon-optimized version of SEQ ID NO:16; a nucleic acid as defined in ii) comprises a nucleic acid sequence selected from a group consisting of any codon-optimized version of SEQ ID NO:17; and wherein a nucleic acid as defined in iii) comprises a nucleic acid sequence selected from a group consisting of any codon-optimized version of SEQ ID NO:18. In one embodiment, said immunogenic composition comprises a nucleic acid comprising a sequence selected from a group consisting of any codon-optimized version of SEQ ID NO:16; a nucleic acid comprising a sequence selected from a group consisting of any codon-optimized version of SEQ ID NO:17; and a nucleic acid comprising a sequence selected from a group consisting of any codon-optimized version of SEQ ID NO:18. In one embodiment, said immunogenic composition comprises SEQ ID NO:32, SEQ ID NO:33 and SEQ ID NO:34. In one embodiment, said immunogenic composition comprises a nucleic acid comprising a sequence selected from a group consisting of any codon-optimized and nucleoside modified version of SEQ ID NO:16; a nucleic acid comprising a sequence selected from a group consisting of any codon-optimized and nucleoside modified version of SEQ ID NO:17; and a nucleic acid comprising a sequence selected from a group consisting of any codon-optimized and nucleoside modified version of SEQ ID NO:18. In one embodiment, said immunogenic composition comprises a nucleic acid comprising SEQ ID NO:86, a nucleic acid comprising SEQ ID NO:87 and a nucleic acid comprising SEQ ID NO:88.

In addition, the immunogenic composition of the disclosure may further comprise one or more nucleic acids encoding an HSV glycoprotein. Accordingly, in one embodiment, said one or more nucleic acid(s) encoding an HSV glycoprotein is/are selected from a group consisting of a) a nucleic acid encoding an HSV glycoprotein D (gD) or an immunogenic fragment thereof, b) a nucleic acid encoding an HSV glycoprotein B (gB) or an immunogenic fragment thereof, and c) a nucleic acid encoding an HSV glycoprotein E (gE) or an immunogenic fragment thereof.

In one embodiment, said HSV gD, said HSV gB and/or said HSV gE is/are an HSV-2 glycoprotein(s). In one embodiment, said HSV gD, said HSV gB and said HSV gE are HSV-2 glycoproteins.

In one embodiment, said one or more nucleic acid(s) encoding an HSV glycoprotein is/are selected from a group consisting of a) a nucleic acid encoding an HSV glycoprotein D (gD) or an immunogenic fragment thereof comprising or consisting of an amino acid sequence which is at least 70%, such as at least 80%, or more identical to the amino acid sequence of SEQ ID NO:11,b) a nucleic acid encoding an HSV glycoprotein B (gB) or an immunogenic fragment thereof comprising or consisting of an amino acid sequence which is at least 70%, such as at least 80%, or more identical to the amino acid sequence of SEQ ID NO:10, and c) a nucleic acid encoding an HSV glycoprotein E (gE) or an immunogenic fragment thereof comprising or consisting of an amino acid sequence selected from a group consisting of SEQ ID NO:4, SEQ ID NO:83, SEQ ID NO:5, SEQ ID NO:21 and any amino acid sequence having at least 70%, such as at least 80%, or more identity to SEQ ID NO:4, SEQ ID NO:83, SEQ ID NO:5 and/or SEQ ID NO:21.

In one embodiment, said nucleic acid as defined in c) encodes an amino acid sequence selected from a group consisting of SEQ ID NO:4, SEQ ID NO:83 and any amino acid sequence having at least 70%, such as at least 80%, or more identity to SEQ ID NO:4 and/or SEQ ID NO:83. In one embodiment, said nucleic acid as defined in c) encodes an amino acid sequence selected from a group consisting of SEQ ID NO:4, SEQ ID NO:5 and any amino acid sequence having at least 70%, such as at least 80%, or more identity to SEQ ID NO:4 and/or SEQ ID NO:5. In one embodiment, said nucleic acid as defined in c) comprises or consist of an amino acid sequence selected from a group consisting of SEQ ID NO:4 and any amino acid sequence having at least 70%, such as at least 80%, or more identity to SEQ ID NO:4.

In one embodiment, said immunogenic fragment of gD as defined in a) encodes an amino acid sequence according to positions 26 to 331 of the amino acid sequence according to SEQ ID NO:11 or an amino acid sequence having at least 70%, such as at least 80%, or more identity to the amino acid sequence according to SEQ ID NO:11. In one embodiment, said immunogenic fragment of gD as defined in a) encodes an amino acid sequence according to positions 26 to 331 of the amino acid sequence according to SEQ ID NO:11.

In one embodiment, the nucleic acid as defined in a), b) and/or c) is selected from the group consisting of DNA and RNA.

In one embodiment, the nucleic acid as defined in a), b) and/or c) is DNA.

In one embodiment, the nucleic acid as defined in a) comprises a nucleic acid sequence according to SEQ ID NO:75 or any sequence having at least 80%, such as at least 85%, such as at least 90%, such as at least 95%, such as at least 98%, such as at least 99% identity to SEQ ID NO:75. In one embodiment, the nucleic acid as defined in b) comprises a nucleic acid sequence according to SEQ ID NO:74 or any sequence having at least 80%, such as at least 85%, such as at least 90%, such as at least 95%, such as at least 98%, such as at least 99% identity to SEQ ID NO:74. In one embodiment, the nucleic acid as defined in c) comprises a nucleic acid sequence selected from a group consisting of SEQ ID NO:73, SEQ ID NO:57, SEQ ID NO:58 or any sequence having at least 80%, such as at least 85%, such as at least 90%, such as at least 95%, such as at least 98%, such as at least 99% identity to SEQ ID NO:54, SEQ ID NO:76 and/or SEQ ID NO:77. In one embodiment, the nucleic acid as defined in a) comprises a nucleic acid sequence according to SEQ ID NO:75 or any sequence having at least 80%, such as at least 85%, such as at least 90%, such as at least 95%, such as at least 98%, such as at least 99% identity to SEQ ID NO:75. In one embodiment, the nucleic acid as defined in b) comprises a nucleic acid sequence according to SEQ ID NO:74 or any sequence having at least 80%, such as at least 85%, such as at least 90%, such as at least 95%, such as at least 98%, such as at least 99% identity to SEQ ID NO:74. In one embodiment, the nucleic acid as defined in c) comprises a nucleic acid sequence selected from a group consisting of SEQ ID NO:73, SEQ ID NO:77 or any sequence having at least 80%, such as at least 85%, such as at least 90%, such as at least 95%, such as at least 98%, such as at least 99% identity to SEQ ID NO:73 and/or SEQ ID NO:77. In one embodiment, the nucleic acid as defined in a) comprises a nucleic acid sequence according to SEQ ID NO:75 or any sequence having at least 80%, such as at least 85%, such as at least 90%, such as at least 95%, such as at least 98%, such as at least 99% identity to SEQ ID NO:75. In one embodiment, the nucleic acid as defined in b) comprises a nucleic acid sequence according to SEQ ID NO:74 or any sequence having at least 80%, such as at least 85%, such as at least 90%, such as at least 95%, such as at least 98%, such as at least 99% identity to SEQ ID NO:74. In one embodiment, the nucleic acid as defined in c) comprises a nucleic acid sequence selected from a group consisting of SEQ ID NO:57, SEQ ID NO:77 or any sequence having at least 80%, such as at least 85%, such as at least 90%, such as at least 95%, such as at least 98%, such as at least 99% identity to SEQ ID NO:57 and/or SEQ ID NO:77. In one embodiment, the nucleic acid as defined in a) comprises a nucleic acid sequence according to SEQ ID NO:75. In one embodiment, the nucleic acid as defined in b) comprises a nucleic acid sequence according to SEQ ID NO:74. In one embodiment, the nucleic acid as defined in c) comprises a nucleic acid sequence according to SEQ ID NO:73, SEQ ID NO:76 or SEQ ID NO:77. In one embodiment, the nucleic acid as defined in c) comprises a nucleic acid sequence according to SEQ ID NO:73 or SEQ ID NO:76. In one embodiment, the nucleic acid as defined in c) comprises a nucleic acid sequence according to SEQ ID NO:76 or SEQ ID NO:77. In one embodiment, the nucleic acid as defined in c) comprises a nucleic acid sequence according to SEQ ID NO:76. In one embodiment, the nucleic acid as defined in a) comprises a nucleic acid sequence according to SEQ ID NO:75. In one embodiment, the nucleic acid as defined in b) comprises a nucleic acid sequence according to SEQ ID NO:74. In one embodiment, the nucleic acid as defined in c) comprises a nucleic acid sequence according to SEQ ID NO:73, SEQ ID NO:76 or SEQ ID NO:77.

In one embodiment, the nucleic acid as defined in c) comprises a nucleic acid sequence according to SEQ ID NO:73 or SEQ ID NO:76. In one embodiment, the nucleic acid as defined in c) comprises a nucleic acid sequence according to SEQ ID NO:76 or SEQ ID NO:77. In one embodiment, the nucleic acid as defined in c) comprises a nucleic acid sequence according to SEQ ID NO:76.

In one embodiment, the nucleic acid as defined in a), b) and/or c) is RNA. In one particular embodiment, the RNA is a messenger RNA (mRNA). Said RNA may be obtained by an RNA manufacturing method, such as by in vitro transcription.

In one embodiment, the nucleic acid as defined in a) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:22 and any codon-optimized version thereof, such as a group consisting of SEQ ID NO:22 and any codon-optimized version thereof. In one embodiment, the nucleic acid as defined in a) comprises a nucleic acid sequence selected from a group consisting of SEQ ID NO:22 and SEQ ID NO:69. In one embodiment, the nucleic acid as defined in a) comprises a nucleic acid sequence selected from any codon-optimized version of SEQ ID NO:22. In one particular embodiment, the nucleic acid as defined in a) comprises a nucleic acid sequence according to SEQ ID NO:69. In one embodiment, the nucleic acid as defined in a) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:12 and any codon-optimized version thereof, such as a group consisting of SEQ ID NO:12 and any codon-optimized version thereof.

In one embodiment, the nucleic acid as defined in b) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:23 and any codon-optimized version thereof, such as a group consisting of SEQ ID NO:23 and any codon-optimized version thereof. In one embodiment, the nucleic acid as defined in b) comprises a nucleic acid sequence selected from a group consisting of SEQ ID NO:23 and SEQ ID NO:68. In one embodiment, the nucleic acid as defined in b) comprises a nucleic acid sequence selected from any codon-optimized version of SEQ ID NO:23. In one particular embodiment, the nucleic acid as defined in b) comprises a nucleic acid sequence according to SEQ ID NO:68.

In one embodiment, the nucleic acid as defined in c) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:24, SEQ ID NO:78, SEQ ID NO:84 and any codon-optimized version thereof, such as a group consisting of SEQ ID NO:24, SEQ ID NO:78, SEQ ID NO:84 and any codon-optimized version thereof. In one embodiment, the nucleic acid as defined in c) comprises a nucleic acid sequence selected from a group consisting of SEQ ID NO:24, SEQ ID NO:78, SEQ ID NO:84, SEQ ID NO:67, SEQ ID NO:79 and SEQ ID NO:81. In one embodiment, the nucleic acid as defined in c) comprises a nucleic acid sequence selected from any codon-optimized version of SEQ ID NO:24, SEQ ID NO:78 and/or SEQ ID NO:84. In one particular embodiment, the nucleic acid as defined in c) comprises a nucleic acid sequence according to SEQ ID NO:67, SEQ ID NO:79 and/or SEQ ID NO:81.

In one embodiment, the nucleic acid as defined in c) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:24, SEQ ID NO:84 and any codon-optimized version thereof, such as a group consisting of SEQ ID NO:24, SEQ ID NO:84 and any codon-optimized version thereof. In one embodiment, the nucleic acid as defined in c) comprises a nucleic acid sequence selected from a group consisting of SEQ ID NO:24, SEQ ID NO:85, SEQ ID NO:67 and SEQ ID NO:81. In one embodiment, the nucleic acid as defined in c) comprises a nucleic acid sequence selected from any codon-optimized version of SEQ ID NO:24 and/or SEQ ID NO:84. In one particular embodiment, the nucleic acid as defined in c) comprises a nucleic acid sequence according to SEQ ID NO:67 and/or SEQ ID NO:81.

In one embodiment, the nucleic acid as defined in c) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:78, SEQ ID NO:84 and any codon-optimized version thereof, such as a group consisting of SEQ ID NO:78, SEQ ID NO:84 and any codon-optimized version thereof. In one embodiment, the nucleic acid as defined in c) comprises a nucleic acid sequence selected from a group consisting of SEQ ID NO:78, SEQ ID NO:84, SEQ ID NO:67 and SEQ ID NO:79. In one embodiment, the nucleic acid as defined in c) comprises a nucleic acid sequence selected from any codon-optimized version of SEQ ID NO:78 and/or SEQ ID NO:84. In one particular embodiment, the nucleic acid as defined in c) comprises a nucleic acid sequence according to SEQ ID NO:67 and/or SEQ ID NO:79.

In one embodiment, the nucleic acid as defined in c) comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:84 and any codon-optimized version thereof, such as a group consisting of SEQ ID NO:84 and any codon-optimized version thereof. In one embodiment, the nucleic acid as defined in c) comprises a nucleic acid sequence selected from a group consisting of SEQ ID NO:84 and SEQ ID NO:67. In one embodiment, the nucleic acid as defined in c) comprises a nucleic acid sequence selected from any codon-optimized version of SEQ ID NO:84. In one particular embodiment, the nucleic acid as defined in c) comprises a nucleic acid sequence according to SEQ ID NO:67.

The term “gD” when used herein may sometimes be referred to as “glycoprotein D”. SEQ ID NO:41 depicts an exemplarily an amino acid sequence of HSV-2 gD. However the term “gD” also encompasses gD polypeptides having an amino acid sequence which shares a certain degree of identity with the amino acid sequence shown in SEQ ID NO:11 and also encompasses polypeptides having mutations relative to the reference sequence shown in SEQ ID NO:11 as described herein. In one embodiment, the gD protein and/or said immunogenic fragment thereof translated from the nucleic acid sequence as defined in a) is able to form a complex with one or more protein selected from a group consisting of UL11, UL16 and UL21. In one embodiment, said complex is a protein complex comprising or consisting of the gD protein or said immunogenic fragment thereof, UL11, UL16 and UL21.

The term “gB” when used herein may sometimes be referred to as “glycoprotein B”. SEQ ID NO:40 depicts an exemplarily amino acid sequence of HSV-2 gB. However the term “gB” also encompasses gB polypeptides having an amino acid sequence which shares a certain degree of identity with the amino acid sequence shown in SEQ ID NO:10 and also encompasses polypeptides having mutations relative to the reference sequence shown in SEQ ID NO:10 as described herein. In one embodiment, the gB protein and/or said immunogenic fragment thereof translated from the nucleic acid sequence as defined in b) is able to form a complex with one or more protein selected from a group consisting of UL11, UL16 and UL21. In one embodiment, said complex is a protein complex comprising or consisting of the gB protein or said immunogenic fragment thereof, UL11, UL16 and UL21.

The term “gE” when used herein may sometimes be referred to as “glycoprotein E”. SEQ ID NO:39 depicts an exemplarily an amino acid sequence of HSV-2 gE, also deposited with NCBI GenBank under accession number AHG54732.1. However the term “gE” also encompasses gE polypeptides having an amino acid sequence which shares a certain degree of identity with the amino acid sequence shown in SEQ ID NO:4 and also encompasses polypeptides having mutations relative to the reference sequence shown in SEQ ID NO:4 as described herein. In one embodiment, the gE protein and/or said immunogenic fragment thereof translated from the nucleic acid sequence as defined in c) is able to bind UL11. In one embodiment, the binding of the gE protein and/or said immunogenic fragment thereof to UL11 facilitates binding of the gE protein and/or said immunogenic fragment to UL16. In one embodiment, the gE protein and/or said immunogenic fragment thereof is able to form a protein complex comprising or consisting of the gE protein and/or said immunogenic fragment thereof, UL11 and UL16. In one embodiment, the gE protein and/or said immunogenic fragment thereof is able to form a protein complex with UL16 and UL21 comprising or consisting of the gE protein and/or said immunogenic fragment thereof, UL16 and UL21. In one embodiment, the gE protein and/or said immunogenic fragment thereof is able to form a protein complex comprising or consisting of the gE protein and/or said immunogenic fragment thereof, UL11, UL16 and UL21.

In one embodiment, said immunogenic fragment of gE according to c) comprises or consists of the cytoplasmic domain (i.e. cytoplasmic tail) of an HSV, such as an HSV-2, gE polypeptide. In one embodiment, said cytoplasmic domain of the gE polypeptide comprises or consists of an amino acid sequence selected from a group consisting of SEQ ID NO:5 and any amino acid sequence having at least 70%, such as at least 80%, or more identity to SEQ ID NO:5.

In one embodiment, the nucleic acid as defined in c) encodes said cytoplasmic domain of the gE polypeptide and comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:78, SEQ ID NO:21 and any codon-optimized version thereof, such as a group consisting of SEQ ID NO:78, SEQ ID NO:21 and any codon-optimized version thereof. In one embodiment, the nucleic acid as defined in c) comprises a nucleic acid sequence selected from a group consisting of SEQ ID NO:78 and SEQ ID NO:79. In one embodiment, the nucleic acid as defined in c) comprises a nucleic acid sequence selected from any codon-optimized version of SEQ ID NO:78. In one embodiment, the nucleic acid as defined in c) comprises a nucleic acid sequence selected from any codon-optimized version of SEQ ID NO:21. In one particular embodiment, the nucleic acid as defined in c) comprises a nucleic acid sequence according to SEQ ID NO:79. In one embodiment, said immunogenic fragment of gE according to c) comprises or consists of the extracellular domain of an HSV, such as an HSV-2, gE polypeptide. In one embodiment, said cytoplasmic domain of the gE polypeptide comprises or consists of an amino acid sequence selected from a group consisting of SEQ ID NO:83 and any amino acid sequence having at least 70%, such as at least 80%, or more identity to SEQ ID NO:83.

In one embodiment, the nucleic acid as defined in c) encodes said extracellular domain of the gE polypeptide and comprises a nucleic acid sequence selected from a group consisting of any nucleic acid sequence having at least 80%, such as at least 85%, such as at least 90%, such as at least 95%, such as at least 98%, such as at least 99% identity to SEQ ID NO:24 and any codon-optimized version thereof, such as a group consisting of SEQ ID NO:24 and any codon-optimized version thereof. In one embodiment, the nucleic acid as defined in c) comprises a nucleic acid sequence selected from a group consisting of SEQ ID NO:24 and SEQ ID NO:81. In one embodiment, the nucleic acid as defined in c) comprises a nucleic acid sequence selected from any codon-optimized version of SEQ ID NO:24. In one particular embodiment, the nucleic acid as defined in c) comprises a nucleic acid sequence according to SEQ ID NO:81.

As appreciated by those skilled in art, one or more nucleoside(s) may be replaced by other naturally modified nucleosides or by synthetic nucleoside analogues during RNA, such as mRNA, manufacturing. Nucleoside modified mRNAs encode the same protein as the protein encoded by the non-nucleoside modified mRNA variant. Certain modified nucleosides are considered to enhance the stability of the mRNA and/or increase the efficiency of its translation which in turn enhances the production of the desired protein in the cell comprising the mRNA. Certain nucleoside modifications are considered to alter the immunogenicity profile of vaccines which comprise such nucleoside modified mRNA, and can be particularly useful to reduce the innate immune response often elicited by non-nucleoside modified mRNA variants. This may be achieved by decreasing the recognition of the mRNA as foreign by pattern recognition receptors which would normally result in the activation of innate immunity. Commonly employed such nucleoside modifications include for example 5-methoxyuridine, which is a modified nucleoside triphosphate (NTP) for incorporation into mRNA using T7 RNA polymerase. Incorporation of 5-methoxyuridine can reduce the immunogenicity of the resulting mRNA. 5-methylcytidine (m5C) is also one example of such nucleoside modifications that involves the addition of a methyl group to cytidine. Additional non-limiting examples are N6-methyladenosine (m6A), which involves the addition of a methyl group to adenosine, 5-methyluridine (m5U), which involves the addition of a methyl group to uridine, 2-thiouridine (s2U), which contains a sulphur group and is known to increase mRNA stability, 2′-0-methylguanosine (m2,2G), which involves the addition of a methyl group to the 2′-oxygen of guanosine, 2′-0-methylcytidine (m2,2C), which involves the addition of a methyl group to the 2′-oxygen of cytidine, 2′-0-methyluridine (m2,2U), which involves the addition of a methyl group to uridine at the 2′-oxygen, and 5-methoxycytidine (m5oC), which contains a methoxy group. Pseudouridine (abbreviated by the Greek letter psi, ψ) is an isomer of the nucleoside uridine in which the uracil is attached via a carbon-carbon instead of a nitrogen-carbon glycosidic bond. Pseudouridine, which is the most abundant RNA modification in cellular RNA, can regulate RNA expression post-transcriptionally and plays a variety of roles in the cell including translation, localization and stabilization of RNA. Without being bound by theory, the present inventors envision that modified nucleosides which have one or more of the above discussed beneficial effects on mRNA stability, translation efficiency and/or immunogenicity profile are advantageous in the context of the present disclosure. As used herein, the term “nucleoside modified” in relation to a nucleic acid sequence is meant to be understood that said nucleic acid sequence comprises at least one nucleoside modification as discussed above.

In one non-limiting example, at least one uridine in a RNA sequence is replaced by pseudouridine. In another non-limiting example, all uridines in a RNA sequence are replaced by pseudouridines. Thus, in one embodiment the nucleic acid as defined in i), ii) and/or iii) and/or the nucleic acid sequence as defined in a), b) and/or c) is a nucleoside modified mRNA comprising one or more modified nucleoside(s). In one embodiment, the nucleoside modified mRNA comprises one or more pseudouridine residue(s). In one embodiment, said one or more modified nucleoside(s) is/are selected from a group consisting of 5-methoxyuridine, 5-methylcytidine (m5C), N6-methyladenosine (m6A), 5-methyluridine (m5U), 2-thiouridine (s2U), 2′-0-methylguanosine (m2,2G), 2′-0-methylcytidine (m2,2C), 2′-0-methyluridine (m2,2U), 5-methoxycytidine (m5oC), 1-methylpseudouridine (m1ψ), 1-methyl-3-(3-amino-5-carboxypropyl)pseudouridine (m1acp3ψ), 2′-0-methylpseudouridine (Wm), 5-methyldihydrouridine (m5D) and (3-methylpseudouridine (m3ψ). In one embodiment, said one or more modified nucleoside(s) is/are modified uridine residue(s). In certain embodiments, said modified uridine residue(s) is/are selected from a group consisting of 5-methoxyuridine, 5-methyluridine (m5U), 2-thiouridine (s2U) and 2′-0-methyluridine (m2,2U). In one embodiment, said one or more modified nucleoside(s) is/are pseudouridine residue(s). In certain embodiments, said pseudouridine residue(s) is/are selected from a group consisting of 1-methylpseudouridine (m1ψ), 1-methyl-3-(3-amino-5-carboxypropyl)pseudouridine (m1acp3ψ), 2′-0-methylpseudouridine (Wm), 5-methyldihydrouridine (m5D) and (3-methylpseudouridine (m3ψ). In one embodiment, said one or more modified nucleoside(s) is/are modified cytidine residue(s). In certain embodiments, said modified cytidine residue(s) is/are selected from a group consisting of 5-methylcytidine (m5C), 2′-0-methylcytidine (m2,2C) and 5-methoxycytidine (m5oC). In one embodiment, said one or more modified nucleoside(s) is/are modified adenosine residue(s). In certain embodiments, said modified adenosine residue(s) is/are N6-methyladenosine(s) (m6A). In one embodiment, said one or more modified nucleoside(s) is/are modified guanosine residue(s). In certain embodiments, said modified guanosine residue(s) is/are 2′-0-methylguanosine(s) (m2,2G). It will be appreciated by those skilled in the art that nucleoside modifications which are not listed above but are expected to result in the above discussed advantageous characteristics of the nucleoside modified mRNA may also be applied according to the present disclosure. For the sake of illustration non-limiting examples of nucleoside modified nucleic acid sequences as defined in i) are SEQ ID NO:26 and 86; nucleoside modified nucleic acid sequences as defined ii) are SEQ ID NO:27 and 87; nucleoside modified nucleic acid sequences as defined iii) are SEQ ID NO:28 and 88; nucleoside modified nucleic acid sequences as defined in a) are SEQ ID NO:12 and 29; and nucleoside modified nucleic acid sequence as defined in c) is SEQ ID NO:13.

Polyadenylation is the addition of a poly-A tail (i.e. polyadenylated 3′-ends) to an RNA transcript, typically an mRNA. A poly-A tail consists of multiple adenosine monophosphates—in other words, it is a stretch of RNA that has only adenine bases. The poly-A tail is known to be advantageous for the nuclear export, translation and/or stability of mRNA. The tail is shortened over time in the cells and when it is short enough, the mRNA is enzymatically degraded. Without being bound by theory, such poly-A tail may thus be beneficial in the context of the present disclosure. Accordingly, in one embodiment, the nucleic acid as defined in i), ii) and/or iii) comprise(s) a poly-A tail. In one particular embodiment, said poly-A tail comprises a nucleic acid sequence according to SEQ ID NO:58.

In molecular genetics, an untranslated region (or UTR) refers to either of two sections, one on each side of a coding sequence on a strand of mRNA. If it is found on the 5′ side, it is called the 5′ UTR (also known as leader sequence) or if it is found on the 3′ side, it is called the 3′ UTR (also known as trailer sequence). Within the 5′ UTR, which is thus upstream from the coding sequence, a sequence region is recognized by the ribosome which allows the ribosome to bind and initiate translation. The 3′ UTR, which is thus found immediately following the translation stop codon, plays a critical role in translation termination as well as post-transcriptional modifications. These regions are thus involved in translation regulation and maintaining mRNA stability. Without being bound by theory, the present inventors envision that such 5′ UTR and/or 3′ UTR region(s) is/are beneficial in the context of the present disclosure. Thus, in one embodiment, the nucleic acid as defined in i), ii) and/or iii) and/or the nucleic acid sequence as defined in a), b) and/or c) comprise a 5′ UTR, such as wherein said 5′ UTR enhances translation. 31. In one embodiment, the 5′ UTR comprises a nucleic acid sequence according to SEQ ID NO:59. In another embodiment, the nucleic acid as defined in i), ii) and/or iii) and/or the nucleic acid sequence as defined in a), b) and/or c) comprise a 3′ UTR, such as wherein said 3′ UTR enhances translation. In one embodiment, the 3′ UTR comprises a nucleic acid sequence according to SEQ ID NO:60. In a particular embodiment, the nucleic acid as defined in i), ii) and/or iii) and/or the nucleic acid sequence as defined in a), b) and/or c) comprise both a 5′ UTR and a 3′ UTR. In another particular embodiment, the nucleic acid as defined in i), ii) and/or iii) comprise(s) both a 5′ UTR comprising a nucleic acid sequence according to SEQ ID NO:59 and a 3′ UTR comprising a nucleic acid sequence according to SEQ ID NO:60.

In view of the above discussed advantages of incorporating a poly-A tail, a 5′ UTR and/or a 3′ UTR into an mRNA, incorporating each of these into one or more mRNA(s) as disclosed herein, may be particularly beneficial. Thus, in one particular embodiment of the present disclosure, the nucleic acid as defined in i) comprises a nucleic acid sequence according to SEQ ID NO:61.

In another particular embodiment, the nucleic acid as defined in ii) comprises a nucleic acid sequence according to SEQ ID NO:62. In yet another particular embodiment, the nucleic acid as defined in iii) comprises a nucleic acid sequence according to SEQ ID NO:63.

In one particular embodiment of the present disclosure, the nucleic acid as defined in a) comprises a nucleic acid sequence according to SEQ ID NO:66. In one particular embodiment of the present disclosure, the nucleic acid as defined in b) comprises a nucleic acid sequence according to SEQ ID NO:65. In one particular embodiment of the present disclosure, the nucleic acid as defined in c) comprises a nucleic acid sequence selected from a group consisting of SEQ ID NO:64, SEQ ID NO:80 and SEQ ID NO:82, such as wherein said nucleic acid as defined in c) comprises a nucleic acid sequence according to SEQ ID NO:64.

The nucleic acid may be nucleoside modified as described herein, for example pseudouridine modified, for example with 1-methyl-pseudouridine modified. In one particular embodiment of the present disclosure all uridine residues are replaced by pseudouridine residues, such as by 1-methyl-pseudouridine, the nucleic acid as defined in i) comprises a nucleic acid sequence according to SEQ ID NO:26. In another particular embodiment, the nucleic acid as defined in ii) comprises a nucleic acid sequence according to SEQ ID NO:27. In yet another particular embodiment, the nucleic acid as defined in iii) comprises a nucleic acid sequence according to SEQ ID NO:28.

mRNA capping, is highly regulated and vital in the creation of stable and mature mRNA, which is able to undergo translation during protein synthesis. An mRNA cap, also known as a five-prime cap (5′ cap) is a specially altered nucleotide on the 5′ end of the mRNA. The present inventors envision that such mRNA cap is beneficial in the context of the present disclosure, considering its advantageous effects on mRNA stability and maturation. Accordingly, in one embodiment, the nucleic acid as defined in i), ii) and/or iii) and/or the nucleic acid sequence as defined in a), b) and/or c) comprise an mRNA cap. Said mRNA cap may for example be an m7GpppG cap, a 3′-0-methyl-m7GpppG cap or an anti-reverse cap analog.

As discussed above, mRNA modifications or mRNA sequence regions which enhance protein production by altering protein translation dynamics are considered beneficial in the context of the present disclosure. Thus, in one embodiment, the nucleic acid as defined in i), ii) and/or iii) and/or the nucleic acid sequence as defined in a), b) and/or c) comprise a cap-independent translational enhancer.

As mentioned above, the present inventors have surprisingly found that the immunogenic composition as disclosed herein elicits a particularly advantageous HSV-2 specific immune response in a subject when administered to said subject. As demonstrated in the appended Examples, said advantageous HSV-2 specific immune response may be evaluated in an in vivo experimental animal model and in comparison to various types of controls, such as naïve controls and controls which have been administered an alternative HSV-2 immunogenic composition known in the prior art. In the following embodiments, such advantageous effect of the immunogenic composition according to the present disclosure is discussed in detail.

The mRNA of the vaccine composition of the present invention encoding HSV polypeptide UL48 preferably encodes an amino acid sequence which is 80% or more identical to the amino acid sequence of SEQ ID NO:6, wherein said HSV UL48 mRNA is capable of eliciting an immune response when administered in the form of a vaccine composition to a subject. Preferably, the mRNA is at least 80% identical to SEQ ID NO:14 or a fragment thereof that is at least 200 nucleotides long.

The term “UL48” when used herein relates to the tegument protein VP16 of HSV. SEQ ID NO:6 depicts exemplarily an amino acid sequence of HSV-2 UL48, also deposited with NCBI GenBank under accession number AHG54712.1. However, the term “UL48” also encompasses UL48 polypeptides having an amino acid sequence which shares a certain degree of identity with the amino acid sequence shown in SEQ ID NO:6 and also encompasses polypeptides having mutations relative to the reference sequence shown in SEQ ID NO:6 as described herein. Accordingly, the term “UL48” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 79%, 78%, 77%, 76%, 75%, or preferably 80% or more compared to the amino acid sequence of SEQ ID NO:6 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 99, 100, 101, 102, 103, 104, 105, 106, 107, 108, 109, 110, 111, 112, 113, 114, 115, 116, 117, 118, 119, 120, 121, 122, 123 or preferably 98 amino acid substitutions, insertions and/or deletions compared to the amino acid sequence of SEQ ID NO:6. Preferred UL48 proteins translated from mRNA of the invention can form a dimer with UL49 or can form a trimer with UL49 and gE or the cytoplasmic tail of gE.

The mRNA of the vaccine composition of the present invention encoding HSV polypeptide UL49 preferably encodes an amino acid sequence which is 62% or more identical to the amino acid sequence of SEQ ID NO:7, wherein said mRNA encoding HSV polypeptide UL49 is capable of eliciting an immune response when administered in the form of a vaccine composition to a subject. Preferably, the mRNA is at least 80% identical to SEQ ID NO:15 or a fragment thereof that is at least 200 nucleotides long.

The term “UL49” when used herein relates to the tegument protein VP22 of HSV. SEQ ID NO:7 depicts exemplarily an amino acid sequence of HSV-2 UL49, also deposited with NCBI GenBank under accession number AKC42813.1. However the term “UL49” also encompasses UL49 polypeptides having an amino acid sequence which shares a certain degree of identity with the amino acid sequence shown in SEQ ID NO:7 and also encompasses polypeptides having mutations relative to the reference sequence shown in SEQ ID NO:7 as described herein. Accordingly, the term “UL49” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 74%, 73%, 72%, 71%, 69%, 68%, 67%, 66%, 65%, 64%, 63%, 61%, 60%, 59%, 58%, 57% or preferably 62% or more compared to the amino acid sequence of SEQ ID NO:2 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 116, 117, 118, 119, 120, 121, 122, 123, 124, 125, 126, 127, 128, 129, 130 or preferably 115 amino acid substitutions, insertions and/or deletions compared to the amino acid sequence of SEQ ID NO:7. Preferred UL49 proteins translated from the mRNA of the invention can form a complex with UL48 and/or gE or the cytoplasmic tail of gE. Accordingly, preferred UL49 proteins can form a dimer with UL48 or gE or the cytoplasmic tail of gE or can form a trimer with UL48 and gE or the cytoplasmic tail of gE.

In a further preferred embodiment of the present invention mRNA encoding the proteins of the multimeric complex comprising HSV polypeptides UL48, UL49 are comprised in the vaccine composition of the present invention. These may also encode a trimer comprising the cytoplasmic domain of HSV polypeptide gE. In this case the multimeric complex translated from the mRNA of the present invention comprises HSV polypeptides UL48, UL49 and the cytoplasmic domain of gE.

Thus, as indicated above, in one embodiment, the immunogenic composition as disclosed herein elicits an HSV-2-specific antibody response in the subject who has been administered the immunogenic composition. In one embodiment, said HSV-2-specific antibody response is an HSV-2-specific serum IgG response. As demonstrated in Example 6 below, in one embodiment, said serum IgG response is detectable in a serum sample obtained from said subject at a dilution of at least about 103, such as at least about 105, such as at least about 106, optionally as measured by ELISA. In one embodiment, said serum IgG response is measured in comparison to serum IgG levels detectable in a control serum sample. The term “control serum sample”, as used herein, refers to a serum sample which may be obtained from a subject who has not been administered any immunogenic composition and/or vaccine composition against HSV, such as HSV-2, nor has been infected with HSV, such as with HSV-2.

Such subject may thus be a naïve control. Said control serum sample may also be obtained from a subject who has been vaccinated against HSV, such as HSV-2, using a vaccine that does not comprise UL11, UL16 and/or UL21 proteins and/or the immunogenic composition according to the present disclosure. It is appreciated by those skilled in the art, that these subjects are considered naïve with respect to the immunogenic composition according to the present disclosure. In one embodiment, said control serum sample is obtained from a naïve control. In certain embodiments, said control serum sample is obtained from a subject prior to the administration of the immunogenic composition as disclosed herein to the subject. Such control serum sample is also known as a “pre-bleed sample”, which may be used as an internal control for evaluating the effect of administering an immunogenic composition to a subject, and is well known in the art.

The present inventors have surprisingly found that the immunogenic composition comprising at least one, such as at least two, such as all three nucleic acid(s) as defined in (i), (ii) and (iii) as disclosed herein is particularly superior in eliciting an HSV-2 antigen-specific T cell response upon restimulation of said subject with one or more HSV-2-specific antigens. It is to be understood that such superior HSV-2 antigen-specific T cell response is also expected to occur upon restimulation by an HSV-2 infection. Accordingly, in one embodiment, the immunogenic composition according to the present disclosure induces an HSV-2 antigen-specific T cell response upon restimulation of said subject with one or more HSV-2-specific antigens. In one embodiment, said one or more HSV-2-specific antigens comprise one or more overlapping peptides, such as a pool of overlapping peptides, derivable from amino acid sequence(s) of UL11, UL16 and/or UL21 proteins. In one embodiment, said one or more overlapping peptides comprise overlapping peptides from each of said UL11, UL16 and UL21 proteins. In one embodiment, said overlapping peptides are 15-mer peptides. In one embodiment, said 15-mer peptides are overlapping by at least 5 amino acids, such as at least 6 amino acids, such as at least 7 amino acids, such as at least 8 amino acids, such as at least 9 amino acids, such as at least 10 amino acids, such as at least 11 amino acids. In one embodiment, said 15-mer peptides are overlapping by 8 to 11 amino acids. In one embodiment, said 15-mer peptides are overlapping by 8 amino acids. In one embodiment, said 15-mer peptides are overlapping by 11 amino acids. The skilled person will appreciate that said one or more HSV-2 specific antigens may be selected based on the experimental model used for testing said HSV-2 antigen-specific T cell response. The skilled person appreciates that inbred mice, for example as demonstrated in Example 18, share the same MHC allele peptides, therefore said one or more HSV-2-specific antigens may be non-overlapping peptides derivable from amino acid sequence(s) of UL11, UL16 and/or UL21 proteins, such as non-overlapping peptides as defined by amino acid sequences according to SEQ ID NO:35-51. As demonstrated in Example 18, using an in vivo mouse model system, in one embodiment, said one or more HSV-2-specific antigens are selected from a group consisting of peptides comprising the amino acid sequences according to SEQ ID NO:35-51. In one embodiment, said one or more HSV-2-specific antigens comprise each of said peptides comprising the amino acid sequences according to SEQ ID NO:35-51. On the other hand, for example, in an outbred mouse population and/or in human, this is not the case and the skilled person is aware to design overlapping peptides covering the full amino acid sequences according to SEQ ID NO:1-3. The skilled person appreciates the concept and is able to perform the design of suitable overlapping peptides without undue burden for evaluating a human immune response based on SEQ ID NO:1-3. In one embodiment, said one or more HSV-2-specific antigens are selected from a group consisting of overlapping peptides designed for ex vivo evaluation of the immune response elicited by the immunogenic composition in the subject administered said immunogenic composition, such as wherein said subject is a human subject. The skilled person is familiar with the concept of peptide design for stimulating an immune response in a subject and knows that it refers to the use of bioinformatical and experimental approaches for selecting suitable peptide sequences for such purpose. These peptides are known to be designed for efficient binding to MHC class I and/or MHC class II proteins and are known to be preferably designed for covering the entire amino acid sequences of antigenic proteins in question. Thus, in one embodiment, said one or more overlapping peptides are selected from a group consisting of peptides designed for ex vivo evaluation of the immune response elicited by the immunogenic composition in the subject administered said immunogenic composition, wherein said peptides are designed based on one or more amino acid sequences according to SEQ ID NO:1-3. In one embodiment, said overlapping peptides cover all three amino acid sequences according to SEQ ID NO:1-3.

As showcased in the appended Examples 12-19, in one embodiment, the HSV-2 antigen-specific T cell response comprises inducing an increase in the number of INF-γ, IL-2 and/or TNF-α secreting HSV-2 antigen-specific T cells. In one embodiment, the HSV-2 antigen-specific T cell response comprises inducing an increase in the number of INF-γ and/or TNF-α secreting HSV-2 antigen-specific T cells. In one embodiment, the HSV-2 antigen-specific T cell response comprises inducing an increase in the number of INF-γ secreting HSV-2 antigen-specific T cells.

As demonstrated in Example 8, said increase in the number of INF-γ, IL-2 and/or TNF-α secreting HSV-2 antigen-specific T cells may be measured in comparison to various types of controls. The skilled person will appreciate that the degree of said increase may be dependent on the in vitro or in vivo experimental model used for testing said HSV-2 antigen-specific T cell response. Moreover, said increase may be assessed in an ex vivo experimental model, for example measured in a sample obtained by biopsy from the subject, such as wherein said subject is a human subject. For example, while an extreme increase may be observed in a laboratory setting, in a real-life situation, this increase may be substantially lower. The skilled person will appreciate that the degree of said increase may be dependent on the medical history of said subject, such as any earlier exposure to an infection by HSV-2 or another HSV variant and/or any vaccination against HSV-2 or another HSV variant. The inventors envision that the degree of said increase is a clinically significant increase in comparison to any relevant control which has not been administered the immunogenic composition as disclosed herein.

The term “control sample”, as used herein, refers to a sample which may be obtained from a subject who has not been administered any immunogenic composition and/or vaccine composition against HSV, such as HSV-2, nor has been HSV, such as HSV-2, infected. Such subject may thus be a naïve control. Said control sample may also be obtained from a subject who has been vaccinated against HSV, such as HSV-2, using a vaccine that does not comprise UL11, UL16 and/or UL21 proteins and/or the immunogenic composition according to the present disclosure. It is appreciated by those skilled in the art, that these subjects are considered naïve with respect to the immunogenic composition according to the present disclosure. In one embodiment, said control sample is obtained from a naïve control. In certain embodiments, said control sample is obtained from a subject prior to the administration of the immunogenic composition as disclosed herein to the subject. Such control sample is also known as a “blank”, which may be used as an internal control for evaluating the effect of administering an immunogenic composition to a subject, and is well known in the art.

In one embodiment, said increase in the number of INF-γ secreting HSV-2 antigen-specific T cells is at least about 2-fold, such as at least about 2.5-fold, such as at least about 5-fold, such as at least about 7.5-fold, such as at least about 10-fold, such as at least about 20-fold, such as at least about 25-fold, such as at least about 50-fold, such as at least about 75-fold, such as at least about 100-fold, such as at least about 200-fold, such as at least about 250-fold, such as at least about 500-fold, such as at least about 750-fold, such as at least about 1000-fold, such as at least about 1500-fold, such as at least about 2000-fold, such as at least about 2500-fold, such as at least about 3000-fold, such as at least about 3500-fold, such as at least about 4000-fold, such as at least about 5000-fold, such as at least about 6000-fold, such as at least about 7000-fold, as measured in comparison to a control sample. In one embodiment, said increase in the number of IL-2 secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, as measured in comparison to said control sample. In one embodiment, said increase in the number of TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, as measured in comparison to said control sample.

The present inventors have surprisingly found and demonstrated in the appended Examples, that the immunogenic composition as disclosed herein is unexpectedly superior in comparison to a composition comprising UL11, UL16 and UL21 proteins, such a composition comprising a multimeric protein complex comprising UL11, UL16 and UL21 proteins, as known in the prior art. Accordingly, in one embodiment, said increase in the number of INF-γ secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, such as at least about 25-fold, such as at least about 30-fold, such as at least about 35-fold, such as at least about 40-fold, such as at least about 50-fold, such as at least about 60-fold, such as at least about 70-fold, as measured in comparison to a control sample obtainable from a subject administered UL11, UL16 and UL21 proteins, such as administered a multimeric protein complex comprising UL11, UL16 and UL21 proteins. In one embodiment, said increase in the number of IL-2 secreting HSV-2 antigen-specific T cells is at least about 1.5-fold, such as at least about 2-fold, such as at least about 2.5-fold, such as at least about 3-fold, as measured in comparison to said control sample obtainable from a subject administered UL11, UL16 and UL21 proteins, such as administered a multimeric protein complex comprising UL11, UL16 and UL21 proteins. In one embodiment, said increase in the number of TNF-α secreting HSV-2 antigen-specific T cells is at least about 1.5-fold, such as at least about 2-fold, such as at least about 2.5-fold, such as at least about 3-fold, such as at least about 3.5-fold, such as at least about 4-fold, as measured in comparison to said control sample obtainable from a subject administered UL11, UL16 and UL21 proteins, such as administered a multimeric protein complex comprising UL11, UL16 and UL21 proteins. As appreciated by the person skilled in the art, the term “control sample obtainable from a subject administered UL11, UL16 and UL21 proteins” differs from the control sample discussed above in that the subject from which the control sample is obtained has been administered a composition comprising UL11, UL16 and UL21 proteins but not the immunogenic composition according to the present disclosure.

As discussed above, the present inventors have surprisingly found that the immunogenic composition as disclosed herein is unexpectedly superior in terms of the HSV-2 antigen-specific T cell response. In particular and as demonstrated in the appended Examples, this superiority is particularly outstanding with respect to inducing multifunctional HSV-2 antigen-specific T cells, especially wherein said multifunctional HSV-2 antigen-specific T cells are INF-γ/IL-2, INF-γ/TNF-α or INF-γ/IL-2/TNF-α secreting HSV-2 antigen-specific T cells. The increase in the number of multifunctional HSV-2 antigen-specific T cells, as discussed in the embodiments below, may be measured in comparison to various controls, such as the control sample and the control sample obtainable from a subject administered UL11, UL16 and UL21 proteins, as explained above. The present inventors have found that this remarkable superiority of the immunogenic composition as disclosed herein is particularly advantageous with respect to the therapeutic and/or prophylactic treatment of, such as the prevention of, a reactivation of a latent HSV-2 infection. Accordingly, in one particular embodiment, said HSV-2 antigen-specific T cell response comprises inducing multifunctional HSV-2 antigen-specific T cells, such as wherein said HSV-2 antigen-specific T cell response comprises inducing an increase in the number of multifunctional HSV-2 antigen-specific T cells. In one embodiment, said multifunctional HSV-2 antigen-specific T cells are selected from a group consisting of INF-γ/IL-2, IL-2/TNF-α, INF-γ/TNF-α and INF-γ/IL-2/TNF-α secreting HSV-2 antigen-specific T cells. In one particular embodiment, said multifunctional HSV-2 antigen-specific T cells are selected from a group consisting of INF-γ/IL-2, INF-γ/TNF-α and INF-γ/IL-2/TNF-α secreting HSV-2 antigen-specific T cells. In one embodiment, said multifunctional HSV-2 antigen-specific T cells are selected from a group consisting of INF-γ/IL-2 and INF-γ/IL-2/TNF-α secreting HSV-2 antigen-specific T cells. In one embodiment, said multifunctional HSV-2 antigen-specific T cells are selected from a group consisting of INF-γ/TNF-α and INF-γ/IL-2/TNF-α secreting HSV-2 antigen-specific T cells. In one particular embodiment, said multifunctional HSV-2 antigen-specific T cells are INF-γ/IL-2/TNF-α secreting HSV-2 antigen-specific T cells. In one embodiment, said multifunctional HSV-2 antigen-specific T cells are IL-2/TNF-α secreting HSV-2 antigen-specific T cells. In one particular embodiment, said multifunctional HSV-2 antigen-specific T cells are INF-γ/IL-2 secreting HSV-2 antigen-specific T cells. In another particular embodiment, said multifunctional HSV-2 antigen-specific T cells are INF-γ/TNF-α secreting HSV-2 antigen-specific T cells.

In one embodiment, said increase in the number of INF-γ/IL-2 secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 100-fold, such as at least about 150-fold, such as at least about 200-fold, such as at least about 250-fold, such as at least about 300-fold, such as at least about 350-fold, such as at least about 400-fold, such as at least about 450-fold, such as at least about 500-fold, as measured in comparison to said control sample.—In one embodiment, said increase in the number of INF-γ/TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 100-fold, such as at least about 150-fold, such as at least about 200-fold, such as at least about 250-fold, such as at least about 300-fold, such as at least about 350-fold, such as at least about 400-fold, such as at least about 450-fold, such as 500-fold, such as at least about 550-fold, such as at least about 600-fold, such as at least about 650-fold, such as at least about 700-fold, such as at least about 750-fold, such as at least about 800-fold, such as at least about 850-fold, such as at least about 900-fold, such as at least about 950-fold, such as at least about 1000-fold, such as at least about 1100-fold, such as at least about 1200-fold, such as at least about 1300-fold, such as at least about 1400-fold, such as at least about 1500-fold, such as at least about 1600-fold, such as at least about 1700-fold, as measured in comparison to said control sample. In one embodiment, said increase in the number of IL-2/TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, as measured in comparison to said control sample. In one embodiment, said increase in the number of INF-γ/IL-2/TNF-α secreting HSV-2 antigen-specific T cells is at least about 10-fold, such as at least about 20-fold, such as at least about 25-fold, such as at least about 50-fold, such as at least about 75-fold, such as at least about 100-fold, such as at least about 125-fold, such as at least about 150-fold, such as at least about 175-fold, such as at least about 200-fold, such as at least about 300-fold, such as at least about 400-fold, such as at least about 450-fold, such as at least about 500-fold, as measured in comparison to said control sample.

In one embodiment, said increase in the number of INF-γ/IL-2 secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 11-fold, such as at least about 12-fold, such as at least about 13-fold, such as at least about 14-fold, such as at least about 15-fold, such as at least about 16-fold, such as at least about 17-fold, such as at least about 18-fold, such as at least about 19-fold, such as at least about 20-fold, such as at least about 25-fold, such as at least about 30-fold, such as at least about 40-fold, such as at least about 50-fold, as measured in comparison to said control sample obtainable from a subject administered UL11, UL16 and UL21 proteins, such as administered a multimeric protein complex comprising UL11, UL16 and UL21 proteins. In one embodiment, said increase in the number of INF-γ/TNF-α secreting HSV-2 antigen-specific T cells is at least about 10-fold, such as at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 55-fold, such as at least about 60-fold, such as at least about 65-fold, such as at least about 70-fold, such as at least about 75-fold, such as at least about 80-fold, such as at least about 100-fold, such as at least about 150-fold, as measured in comparison to said control sample obtainable from a subject administered UL11, UL16 and UL21 proteins, such as administered a multimeric protein complex comprising UL11, UL16 and UL21 proteins. In one embodiment, said increase in the number of INF-γ/IL-2/TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 10-fold, such as at least about 15-fold, as measured in comparison to said control sample obtainable from a subject administered UL11, UL16 and UL21 proteins, such as administered a multimeric protein complex comprising UL11, UL16 and UL21 proteins.

As demonstrated in Example 19, the present inventors have surprisingly found that the immunogenic composition as disclosed herein is particularly superior in inducing HSV-2 antigen-specific INF-γ producing CD4+ and CD8+ T cells upon restimulation of the subject who has been administered the composition with one or more HSV-2-specific antigens—as discussed above. This surprising effect appears to be particularly outstanding in case of CD8+ T cells. Accordingly, in one embodiment of the present disclosure, the immunogenic composition as disclosed herein induces HSV-2 antigen-specific INF-γ producing CD4+ and/or CD8+ T cells upon restimulation of the subject with one or more HSV-2-specific antigens. As discussed above, the induction of said HSV-2 antigen-specific INF-γ producing CD4+ and/or CD8+ T cells may be measured in comparison to various controls. Using various types of controls, the present inventors have surprisingly found and demonstrated in the appended Examples, that the immunogenic composition as disclosed herein is superior in inducing HSV-2 antigen-specific INF-γ producing CD4+ and/or CD8+ T cells in comparison to both naïve controls as well as controls administered a composition comprising UL11, UL16 and UL21 proteins. Thus, in one embodiment, the immunogenic composition as disclosed herein induces an increase in the number of HSV-2 antigen-specific INF-γ producing CD4+ and/CD8+ T cells upon restimulation of the subject with one or more HSV-2-specific antigens in comparison to the control sample. In another embodiment, the immunogenic composition as disclosed herein induces an increase in the number of HSV-2 antigen-specific INF-γ producing CD4+ and/CD8+ T cells upon restimulation of the subject with one or more HSV-2-specific antigens in comparison to the control sample obtainable from a subject administered UL11, UL16 and UL21 proteins, such as administered a multimeric protein complex comprising UL11, UL16 and UL21 proteins.

In one embodiment, said increase in the number of HSV-2 antigen-specific INF-γ producing CD4+ T cells is at least about 2-fold, such as at least about 3-fold, such as at least about 4-fold in comparison to the control sample. In one embodiment, said increase in the number of HSV-2 antigen-specific INF-γ producing CD8+ T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 11-fold, such as at least about 12-fold in comparison to the control sample. In one embodiment, said increase in the number of HSV-2 antigen-specific INF-γ producing CD8+ T cells is at least about 5-fold, such as at least about 6-fold, such as at least about 7-fold, such as at least about 8-fold, such as at least about 9-fold, such as at least about 10-fold, such as at least about 11-fold, such as at least about 12-fold in comparison to the control sample. In one embodiment, said increase in the number of HSV-2 antigen-specific INF-γ producing CD8+ T cells is at least about 7-fold, such as at least about 8-fold, such as at least about 9-fold, such as at least about 10-fold, such as at least about 11-fold, such as at least about 12-fold in comparison to the control sample.

In one embodiment, said increase in the number of HSV-2 antigen-specific INF-γ producing CD4+ T cells is at least about 1.5-fold, such as at least about 2-fold, such as at least about 2.5-fold in comparison to the control sample obtainable from a subject administered UL11, UL16 and UL21 proteins, such as administered a multimeric protein complex comprising UL11, UL16 and UL21 proteins. In one embodiment, said increase in the number of HSV-2 antigen-specific INF-γ producing CD8+ T cells is at least about 1.5-fold, such as at least about 2-fold, such as at least about 3-fold, such as at least about 4-fold, such as at least about 5-fold, such as at least about 6-fold, such as at least about 7-fold in comparison to the control sample to a control sample obtainable from a subject administered UL11, UL16 and UL21 proteins, such as administered a multimeric protein complex comprising UL11, UL16 and UL21 proteins. In one embodiment, said increase in the number of HSV-2 antigen-specific INF-γ producing CD8+ T cells is at least about 4-fold, such as at least about 5-fold, such as at least about 6-fold, such as at least about 7-fold in comparison to the control sample to a control sample obtainable from a subject administered UL11, UL16 and UL21 proteins, such as administered a multimeric protein complex comprising UL11, UL16 and UL21 proteins. In one embodiment, said increase in the number of HSV-2 antigen-specific INF-γ producing CD8+ T cells is at least about 5-fold, such as at least about 6-fold, such as at least about 7-fold in comparison to the control sample to a control sample obtainable from a subject administered UL11, UL16 and UL21 proteins, such as administered a multimeric protein complex comprising UL11, UL16 and UL21 proteins. In one embodiment, said HSV-2 antigen-specific INF-γ producing CD8+ T cells correspond to at least about 10%, such as at least about 12%, such as at least about 13%, such as at least about 14% of the total pool of CD8+ T cells in the spleen of the subject administered said composition. In one embodiment, said HSV-2 antigen-specific INF-γ producing CD8+ T cells correspond to about 14% of the total pool of CD8+ T cells in the spleen of the subject administered said composition. The skilled person appreciates that the proportion of INF-γ producing CD8+ T cells in the spleen may be seen as representative of the entire pool of INF-γ producing CD8+ T cells in said subject. Similarly, a peripheral blood sample may be used for such analysis. Thus, in one embodiment said HSV-2 antigen-specific INF-γ producing CD8+ T cells correspond to at least about 10%, such as at least about 12%, such as at least about 13%, such as at least about 14% of the total pool of CD8+ T cells in the subject administered said composition. In one embodiment, said HSV-2 antigen-specific INF-γ producing CD8+ T cells correspond to about 14% of the total pool of CD8+ T cells in the subject administered said composition. In one embodiment, said HSV-2 antigen-specific INF-γ producing CD8+ T cells correspond to at least about 10%, such as at least about 12%, such as at least about 13%, such as at least about 14% of the total pool of CD8+ T cells in a peripheral blood sample from the subject administered said composition. In one embodiment, said HSV-2 antigen-specific INF-γ producing CD8+ T cells correspond to about 14% of the total pool of CD8+ T cells in a peripheral blood sample from the subject administered said composition.

As appreciated by those skilled in the art, various routinely applied methods and laboratory techniques in the field of molecular and cell biology can be applied for evaluating the above discussed HSV-2 antigen-specific T cell response. The skilled person is aware of such methods and techniques and is able to employ those in the context discussed herein without undue burden. As described in the appended examples, one suitable approach is using Flurospot analysis. Thus, in one embodiment, said HSV-2 antigen-specific T cell response is measured by a Fluorospot analysis.

As used herein the term “sequence identity” or “% identity” refers to the percentage of residue matches between at least two polypeptide sequences aligned using a standardized algorithm.

Such an algorithm may insert, in a standardized and reproducible way, gaps in the sequences being compared in order to optimize alignment between two sequences, and therefore achieve a more meaningful comparison of the two sequences. For purposes of the present invention, the sequence identity between two amino acid sequences or between two nucleotide sequences is determined using the NCBI BLAST program version 2.3.0 (Jan. 13, 2016) (Altschul et al., Nucleic Acids Res. (1997) 25:3389-3402). Sequence identity of two amino acid sequences can be determined with blastp set at the following parameters: Matrix: BLOSUM62, Word Size: 3; Expect value: 10; Gap cost: Existence=11, Extension=1; Compositional adjustments: Conditional compositional score matrix adjustment.

The term “% identity” may for example be calculated as follows. The query sequence is aligned to the target sequence using the CLUSTAL W algorithm (Thompson et al, Nucleic Acids Research, 22: 4673-4680 (1994)). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared, and the percentage of positions in the query sequence that have identical correspondences in the target sequence is reported as % identity.

The present inventors found that the immunogenic composition as disclosed herein is surprisingly advantageous for a vaccine composition against an HSV-2 infection. Especially, the inventors have found that a vaccine composition comprising the above described immunogenic composition elicits a superior therapeutic effect, such as a superior protective effect, in comparison to known HSV-2 vaccines in the prior art.

The skilled person is aware of the fact that in order to elicit an immune response in a subject, an agent with adjuvant properties may be provided to said subject together with immunogenic compositions as disclosed herein. Thus, in one embodiment, said immunogenic composition as described herein further comprises an agent with adjuvant effect. In particular, the agent with an adjuvant effect may be present in an immune-effective amount. It will be appreciated that the immunogenic compositions as disclosed herein, may be useful as a medicament.

Thus, in another aspect of the present disclosure, there is provided a vaccine composition comprising an immunogenic composition as defined herein and a pharmaceutically acceptable carrier or excipient. The vaccine composition preferably comprises a therapeutically effective amount of the, nucleic acids, such as mRNAs, of the invention as defined in i), ii) and iii). Said vaccine may be used in the therapeutic and/or prophylactic treatment of an HSV-2 infection as described above for the immunogenic composition. In one embodiment, said vaccine composition further comprises an agent with adjuvant effect. In one embodiment, said vaccine composition further comprises an immune-effective amount of an agent with adjuvant effect.

As used herein, the term “immune-effective” refers a sufficient amount of an adjuvant to increase the vaccine's immunogenicity to a level high enough to effectively vaccinate a typical patient. As discussed above, an immunogenic composition as disclosed herein and/or a vaccine composition as disclosed herein may comprise an agent with adjuvant effect in an amount that is immuno-effective. Suitably, said adjuvant stimulates systemic or mucosal immunity. The skilled person is aware of suitable adjuvants. Non-limiting examples of suitable adjuvant in the context of the present disclosure include polymers of acrylic or methacrylic acid, maleic anhydride and alkenyl derivative polymers, immunostimulating sequences (ISS), an oil in water emulsion, cation lipids containing a quaternary ammonium salt, cytokines, aluminum hydroxide or aluminum phosphate, saponin or nanoparticles or any combinations or mixtures thereof.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, in Alfonso R Gennaro, Remington: The Science and Practice of Pharmacy. 20th edition, ISBN: 0683306472).

The inventors envision that there are several manners suitable for the delivery of the vaccine composition as disclosed herein and the vaccine composition may be formulated according to the chosen alternative. The skilled person is aware of potential manners suitable for the delivery of a vaccine composition comprising nucleic acids, for example, based on Hou et al, Nature Review Materials, 6: 1078-1094 (2021), Kowalski et al, Molecular Therapy, 27(4): 710-728 (2019), Kimura et al, Molecular Therapy, 31(8): 2360-2375 (2023) and Pardi et al, Nature Reviews, Drug Discovery, 17(4): 261-279 (2018). Accordingly, in one embodiment of the second aspect of the disclosure, the immunogenic composition is encapsulated, attached to a carrier surface or is formulated for delivery to said subject by electroporation. In one embodiment, said immunogenic composition is encapsulated, such as encapsulated in a particle. In one embodiment, said particle is made of a lipid, a polymer, cholesterol and/or a cell penetrating peptide. In one embodiment, said particle is a lipid particle and/or a liposome. In one embodiment and as demonstrated in the appended examples, said particle is a nanoparticle, such as a lipid nanoparticle.

The vaccine composition of the present disclosure is formulated in a form suitable for physiological administration. In certain embodiments, the vaccine composition is formulated for intramuscular administration, subcutaneous administration, intradermal administration, intranasal administration, intravaginal administration, intrarectal administration or topical administration. In one embodiment, the vaccine composition is formulated for intramuscular administration or subcutaneous administration. In one embodiment, the vaccine composition is formulated for subcutaneous administration. In one particular embodiment, the vaccine composition is formulated for intramuscular administration. In one embodiment, the vaccine composition is formulated for injection.

The vaccine composition as disclosed herein comprises the immunogenic composition in an effective amount and as demonstrated in the appended Examples, is capable of eliciting an HSV-2 specific immune response in the subject when administered to said subject as discussed above. The skilled person will appreciate that the embodiments discussed above in relation to the previously discussed aspect relation to the immunogenic composition of the present disclosure, are equally relevant and applicable to this related aspect disclosed herein. This particularly applies to embodiments relation to the components present in the composition as well as relating to the HSV-2 specific immune response including the HSV-2-specific antibody response, the HSV-2 antigen-specific T cell response upon restimulation of said subject with one or more HSV-2-specific antigens and the induction of multifunctional HSV-2 antigen-specific T cells. For the sake of brevity these will not be repeated in relation to the this related aspect or will only be briefly mentioned.

The term “immune response” refers to the ability to induce a humoral and/or cell mediated immune response, preferably but not only in vivo. A humoral immune response comprises a B-cell mediated antibody response. A cell mediated immune response comprises a T-cell mediated immune response, including but not limited to CD4+ T-cells and CD8+ T-cells. The ability of an antigen to elicit immune responses is called immunogenicity, which can be humoral and/or cell-mediated immune responses. An immune response of the present invention is preferably an immune response against HSV, in particular against HSV-2, and even more preferably an immune response against an HSV infection, in particular against HSV-2, in a subject.

In one embodiment, said subject is human subject.

The ability to induce a humoral and/or cell mediated immune response in vivo can be determined using a guinea pig model of genital HSV-2 infection, which accurately mirrors the disease in humans and represents a system to examine pathogenesis and therapeutic efficacy of candidate antiviral compounds and vaccines. It also serves as an ideal system to address the nature of both genital-resident and neural tissue-resident immune memory. Genital infection of guinea pigs results in a self-limiting vulvovaginitis with neurologic manifestations mirroring those found in human disease. Primary disease in female guinea pigs involves virus replication in genital epithelial cells which is generally limited to eight days. During this time, virus reaches sensory nerve endings and is transported by retrograde transport to cell bodies in the sensory ganglia and autonomic neurons in spinal cords. Following a brief period of acute replication at this site, the immune system usually resolves acute virus replication by day 15 post inoculation and the virus is maintained as a lifelong, latent infection of sensory neurons. Following recovering from primary HSV-2 genital infection guinea pigs experience episodic spontaneous recurrent infection and disease. HSV-2 recurrences may manifest as clinically apparent disease with erythematous and/or vesicular lesions on the perineum or as asymptomatic recurrences characterized by shedding of virus from the genital tract. Vaccine efficacy may for example be assessed using the guinea pig genital infection model. Animals may be infected intravaginally with 5×10¹ PFU, 5×10² PFU, 5×10³ PFU, 5×10⁴ PFU, 5×10⁶ PFU, 5×10⁷ PFU, 5×10⁸ PFU, or 5×10⁹ PFU and preferably 5×10⁵ PFU of HSV-2 (e.g. strain MS). Animals may be immunized prior or post infection one, two, three, four, five or more times. Preferably, at day 15 post infection animals were immunized twice with 15 days interval. In general, any suitable route of administration may be used for immunization. However, animals are preferably immunized intramuscularly. Possible control groups are either mock-immunized with adjuvant-only (e.g. CpG 100 μg/Alum 150 μg) or with PBS (both negative controls), or with the HSV-2 dl5-29 mutant virus strain (positive control). Groups that are immunized with vaccine candidates combined with the adjuvant may receive a dose of 0.1 μg, 0.5 μg, 1 μg, 2 μg, 3 μg, 4 μg, 5 μg, 10 μg, 15 μg, 25 μg, 30 μg, 35 μg, 40 μg, 50 μg, 60 μg, 70 μg, 80 μg, 90 μg, 100 μg, 150 μg, 200 μg and preferably 20 μg of the respective mRNA in each immunization round. As a read out vaginal swabs can be collected for evaluation of the frequency and magnitude of recurrent virus shedding, e.g. from day 0 post infection to day 200, day 1 post infection to day 180, day 3 post infection to day 160, day 5 post infection to day 140, day 7 post infection to day 120, day 10 post infection to day 100, day 12 post infection to day 90. Vaginal swabs can be collected every 1, 2, 3, 4, 5, 6, 7, 8, 9, or 10 days. Preferably, vaginal swabs are collected every day, from day 15 post infection to day 85. In the same time interval the severity (scores 0 to 4) and duration of recurrent genital herpetic lesions are scored daily. Preferably, at the end of study the antibody responses as well as the CD4+ and CD8+ T-cell responses are determined.

A variety of routes are applicable for administration of the vaccine composition of the present invention, including, but not limited to, orally, topically, transdermally, subcutaneously, intravenously, intraperitoneally, intramuscularly or intraocularly. However, any other route may readily be chosen by the person skilled in the art if desired.

The exact dose of the immunogenic composition and/or the vaccine composition of the invention which is administered to a subject may depend on the purpose of the treatment (e.g. treatment of acute disease vs. prophylactic vaccination), route of administration, age, body weight, general health, sex, diet, time of administration, drug interaction and the severity of the condition, and will be ascertainable with routine experimentation by those skilled in the art. The administered dose is preferably an effective dose, i.e. effective to elicit an immune response. In a preferred embodiment, the immunogenic composition and/or vaccine composition is administered in two doses of 50-150 μg, preferably 100 μg each 14-42 days apart, preferably 28 days apart.

The immunogenic composition and/or vaccine composition may be administered at one or several doses, such as two doses, of less than about 200 μg, such as less than about 150 μg, such as less than about 100 μg, such as is between about 10 μg and about 100 μg.

The vaccine composition of the present invention may be administered to the subject one or more times, such as 2, 3, 4, 5, 6, 7, 8, 9, 10, or more times.

The “subject” as used herein relates to an animal, preferably a mammal, which can be, for instance, a mouse, rat, guinea pig, hamster, rabbit, dog, cat, or primate. Preferably, the subject is a human. However, the term “subject” also comprises cells, preferably mammalian cells, even more preferred human cells. Such a cell may be an immune cell, preferably a lymphocyte.

The mRNA of the vaccine composition of the present invention encoding HSV polypeptide UL11 preferably encodes an amino acid sequence which is 75% or more identical to the amino acid sequence of SEQ ID NO:1, wherein said HSV UL11 mRNA is capable of eliciting an immune response when administered in the form of a vaccine composition to a subject. Preferably, the mRNA is at least 80% identical to SEQ ID NO:16 or a fragment thereof that is at least 200 nucleotides long.

The term “UL11” when used herein relates to a tegument protein of HSV. SEQ ID NO:1 depicts exemplarily an amino acid sequence of HSV-2 UL11, also deposited with NCBI GenBank under accession number AHG54674.1. However, the term “UL11” also encompasses UL11 polypeptides having an amino acid sequence which shares a certain degree of identity with the amino acid sequence shown in SEQ ID NO:1 and also encompasses polypeptides having mutations relative to the reference sequence shown in SEQ ID NO:1 as described herein. As further discussed below, the skilled person is aware that certain degree of mismatch between two amino acid sequences has no significant bearing on the structure and function of a protein comprising any of the two amino acid sequences. It will be appreciated that such degree of mismatch is reasonable and may be introduced into the herein disclosed exemplary amino acid sequences without any impairment of the structure and function of proteins encoded by these exemplary amino acid sequences. In one embodiment, the UL11 protein and/or said immunogenic fragment thereof translated from the nucleic acid as defined in i) is able to form a complex with one or more protein(s) selected from a group consisting of UL16, UL21, an HSV glycoprotein, such as an HSV-2 glycoprotein E, and an immunogenic fragment thereof, such as a cytoplasmic tail of gE. In one embodiment, the UL11 protein and/or said immunogenic fragment thereof is able to bind to the gE protein and/or said immunogenic fragment thereof. In one embodiment, the binding of the UL11 protein and/or said immunogenic fragment thereof to the gE protein and/or said immunogenic fragment thereof facilitates the binding of the gE protein and/or said immunogenic fragment thereof to UL16. In one embodiment, the UL11 protein and/or said immunogenic fragment thereof is able to form a protein complex comprising or consisting of UL11, UL16 and UL21 or UL11, UL16 and gE or the immunogenic fragment of gE, such as the cytoplasmic tail of gE. In one embodiment, the UL11 protein and/or said immunogenic fragment thereof is able to form a protein complex comprising or consisting of UL11, UL16, UL21 and gE or the immunogenic fragment of gE, such as the cytoplasmic tail of gE.

Accordingly, the term “UL11” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 74%, 73%, 72%, 71%, 70% or preferably 75% or more compared to the amino acid sequence of SEQ ID NO:1 or polypeptides having up to 1, 2, 3, 4, 5, 6, 7, 8, 9, 10, 11, 12, 13, 14, 15, 16, 17, 18, 19, 20, 21, 22, 23, 25, 26, 27, 28, 29 or preferably 24 amino acid substitutions, insertions and/or deletions compared to the amino acid sequence of SEQ ID NO:1. In a particular embodiment, said UL11 protein of HSV-2 comprises or consists of an amino acid sequence according to SEQ ID NO:1. Preferred UL11 proteins translated from the mRNAs of the invention can form a complex with UL16, UL21 and/or gE or the cytoplasmic tail of gE. Accordingly, preferred UL11 proteins translated from the mRNAs of the invention can form a dimer with UL16 or gE or the cytoplasmic tail of gE, can form a trimer with UL16 and UL21 or with UL16 and gE or the cytoplasmic tail of gE and/or can form a tetramer with UL16, UL21 and gE or the cytoplasmic tail of gE.

The mRNA of the vaccine composition of the present invention encoding HSV polypeptide UL16 preferably encodes an amino acid sequence which is 75% or more identical to the amino acid sequence of SEQ ID NO:2, wherein said mRNA encoding HSV polypeptide UL16 is capable of eliciting an immune response when administered in the form of a vaccine composition to a subject. Preferably, the mRNA is at least 80% identical to SEQ ID NO:17 or a fragment thereof that is at least 200 nucleotides long.

The term “UL16” when used herein relates to a tegument protein of HSV. SEQ ID NO:2 depicts exemplarily an amino acid sequence of HSV-2 UL16, also deposited with NCBI GenBank under accession number AHG54679.1. However, the term “UL16” also encompasses UL16 polypeptides having an amino acid sequence which shares a certain degree of identity with the amino acid sequence shown in SEQ ID NO:2 and also encompasses polypeptides having mutations relative to the reference sequence shown in SEQ ID NO:2 as described herein. In one embodiment, the UL16 protein and/or said immunogenic fragment thereof translated from the nucleic acid as defined in ii) is able to form a complex with one or more protein(s) selected from a group consisting of UL11, UL21, gE and the immunogenic fragment of gE, such as the cytoplasmic tail of gE. In one embodiment, the UL16 protein and/or said immunogenic fragment thereof is able to form a protein complex comprising or consisting of UL16 and gE or the immunogenic fragment of gE, such as the cytoplasmic tail of gE; or UL16 and UL21. In one embodiment, the UL16 protein and/or said immunogenic fragment thereof is able to form a protein complex comprising or consisting of UL11, UL16 and gE or the immunogenic fragment of gE, such as the cytoplasmic tail of gE. In one embodiment, the UL16 protein and/or said immunogenic fragment thereof is able to form a protein complex comprising or consisting of UL11, UL16 and UL21. In one embodiment, the UL16 protein and/or said immunogenic fragment thereof is able to form a protein complex comprising or consisting of UL11, UL16, UL21 and gE or the immunogenic fragment of gE, such as the cytoplasmic tail of gE. Accordingly, the term “UL16” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 71%, 70%, 69%, 68%, 67% or preferably 72% or more compared to the amino acid sequence of SEQ ID NO:2 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 105, 106, 107, 108, 109, 110, 111, 112, 113, 114, 115, 116, 117, 118, 119, 120, 121, 122, 123, or preferably 104 amino acid substitutions, insertions and/or deletions compared to the amino acid sequence of SEQ ID NO:2. In a particular embodiment, said UL16 protein of HSV-2 comprises or consists of an amino acid sequence according to SEQ ID NO:2. Preferred UL16 proteins translated from mRNAs of the invention can form a complex with UL11, UL21 and/or gE or the cytoplasmic tail of gE. Accordingly, preferred UL16 proteins translated from mRNAs of the invention can for a dimer with UL21 or UL11, can form a trimer with UL11 and UL21 and/or can form a tetramer with UL11, UL21 and gE or the cytoplasmic tail of gE.

The mRNA of the vaccine composition of the present invention encoding HSV polypeptide UL 21 preferably encodes an amino acid sequence which is 80% or more identical to the amino acid sequence of SEQ ID NO:3, wherein said mRNA encoding HSV polypeptide UL21 is capable of eliciting an immune response when administered in the form of a vaccine composition to a subject. Preferably, the mRNA is at least 80% identical to SEQ ID NO:18 or a fragment thereof that is at least 200 nucleotides long.

The term “UL21” when used herein relates to a tegument protein of HSV. SEQ ID NO:3 depicts exemplarily an amino acid sequence of HSV-2 UL21, also deposited with NCBI GenBank under accession number AHG54684.1. However the term “UL21” also encompasses UL21 polypeptides having an amino acid sequence which shares a certain degree of identity with the amino acid sequence shown in SEQ ID NO:3 and also encompasses polypeptides having mutations relative to the reference sequence shown in SEQ ID NO:3 as described herein. In one embodiment, the UL21 protein and/or said immunogenic fragment thereof translated from the nucleic acid as defined in iii) is able to form a complex with one or more protein(s) selected from a group consisting of UL11, UL16, a gE and the immunogenic fragment of gE, such as the cytoplasmic tail of gE. In one embodiment, the UL21 protein and/or said immunogenic fragment thereof is able to form a protein complex comprising or consisting of UL16 and UL21. In one embodiment, the UL21 protein and/or said immunogenic fragment thereof is able to form a protein complex comprising or consisting of UL16, UL21 and gE or the immunogenic fragment of gE, such as the cytoplasmic tail of gE. In one embodiment, the UL21 protein and/or said immunogenic fragment thereof is able to form a protein complex comprising or consisting of UL11, UL16 and UL21. In one embodiment, the UL21 protein and/or said immunogenic fragment thereof is able to form a protein complex comprising or consisting of UL11, UL16, UL21 and gE or the immunogenic fragment of gE, such as the cytoplasmic tail of gE. Accordingly, the term “UL21” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 79%, 78%, 77%, 76%, 75% or preferably 80% or more compared to the amino acid sequence of SEQ ID NO:3 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, or preferably 134 amino acid substitutions, insertions and/or deletions compared to the amino acid sequence of SEQ ID NO:3. In a particular embodiment, said UL21 protein of HSV-2 comprises or consists of an amino acid sequence according to SEQ ID NO:3. Preferred UL21 proteins translated from mRNAs of the invention can form a complex with UL11, UL16 and/or gE or the cytoplasmic tail of gE. Accordingly, preferred UL21 proteins can for a dimer with UL16, can form a trimer with UL11 and UL16 and/or can form a tetramer with UL11, UL16 and gE or the cytoplasmic tail of gE.

As mentioned herein, the mRNA encoding the proteins of the multimeric complex comprising HSV polypeptides UL11, UL16, UL21 may further comprise mRNA encoding the HSV glycoprotein gE. In this case the multimeric complex translated from the mRNA of the present invention comprises HSV polypeptides UL11, UL16, UL21, and gE.

The HSV polypeptide UL31 encoded by the mRNA of the vaccine composition of the present invention preferably comprises an amino acid sequence which is 85% or more identical to the amino acid sequence of SEQ ID NO:8, wherein said mRNA encoding the HSV polypeptide UL31 is capable of eliciting an immune response when administered in the form of a vaccine composition to a subject. Preferably, the mRNA is at least 80% identical to SEQ ID NO:19 or a fragment thereof that is at least 200 nucleotides long.

The term “UL31” when used herein relates to the virion egress protein of HSV. SEQ ID NO:8 depicts exemplarily an amino acid sequence of HSV-2 UL31, also deposited with NCBI GenBank under accession number AHG54695.1. However, the term “UL31” also encompasses UL31 polypeptides having an amino acid sequence which shares a certain degree of identity with the amino acid sequence shown in SEQ ID NO:8 and also encompasses polypeptides having mutations relative to the reference sequence shown in SEQ ID NO:8 as described herein.

Accordingly, the term “UL31” encompasses polypeptides having an amino acid sequence identity of 99%, 98%, 97%, 96%, 95%, 94%, 93%, 92%, 91%, 90%, 89%, 88%, 87%, 86%, 84%, 83%, 82%, 81%, 80%, or preferably 85% or more compared to the amino acid sequence of SEQ ID NO:1 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61 or preferably 46 amino acid substitutions, insertions and/or deletions compared to the amino acid sequence of SEQ ID NO:8. Preferred UL31 proteins translated from the mRNAs of the invention can form a dimer with UL34.

The HSV polypeptide UL34 encoded by the mRNA of the vaccine composition the present invention preferably comprises an amino acid sequence which is 70% or more identical to the amino acid sequence of SEQ ID NO:9, wherein said HSV mRNA encoding the polypeptide UL34 is capable of eliciting an immune response when administered in the form of a vaccine composition to a subject. Preferably, the mRNA is at least 80% identical to SEQ ID NO:20 or a fragment thereof that is at least 200 nucleotides long.

The term “UL34” when used herein relates to the virion egress protein of HSV. SEQ ID NO:9 depicts exemplarily an amino acid sequence of HSV-2 UL34, also deposited with NCBI GenBank under accession number AHG54698.1. However the term “UL34” also encompasses UL34 polypeptides having an amino acid sequence which shares a certain degree of identity with the amino acid sequence shown in SEQ ID NO:9 and also encompasses polypeptides having mutations relative to the reference sequence shown in SEQ ID NO:9 as described herein.

Accordingly, the term “UL34” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75% 74%, 73%, 72%, 71%, 69%, 68%, 67%, 66%, 65% or preferably 70% or more compared to the amino acid sequence of SEQ ID NO:2 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 76, 77, 78, 79, 80, 81, 82, 83, 84, 85, 86, 87, 88, or preferably 75 amino acid substitutions, insertions and/or deletions compared to the amino acid sequence of SEQ ID NO:9. Preferred UL34 proteins translated from the mRNAs of the invention can for a dimer with UL31.

As stated, each mRNA of the invention, may encode a protein containing mutations, such as insertions, deletions and substitutions relative to the reference sequences shown in SEQ ID NO:1 (UL11), SEQ ID NO:2 (UL16), SEQ ID NO:3 (UL21), SEQ ID NO:4 (gE), SEQ ID NO:5 (cytoplasmic domain of gE), SEQ ID NO:6 (UL48), SEQ ID NO:7 (UL49), SEQ ID NO:8 (UL31) and SEQ ID NO:9 (UL34).

In a further preferred embodiment of the present invention, the vaccine composition comprising mRNAs encoding structural HSV polypeptides described above may also encode one or several HSV glycoproteins. Preferred glycoproteins are gE, gB and gD or fragments thereof.

The mRNA encoding the HSV glycoprotein gE of the vaccine composition the present invention preferably encodes an amino acid or an immunogenic fragment thereof which is 70% or more identical to the amino acid sequence of SEQ ID NO:4. Preferably, the mRNA is at least 80% identical to SEQ ID NO:24 or a fragment thereof that is at least 200 nucleotides long.

The term “ICP4” when used herein may refer to the major viral transcription factor 4 of HSV, e.g., deposited with NCBI GenBank under accession number QIH12398.1 (Version 8 Mar. 2020), and having SEQ ID NO:31 herein. However the term “ICP4” also encompasses ICP4 polypeptides having an amino acid sequence which shares a certain degree of identity with the amino acid sequence shown in SEQ ID NO:31 and also encompasses polypeptides having mutations relative to the reference sequence shown in SEQ ID NO:31 as described herein.

Accordingly, the term “ICP4” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO:31 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO:31. Preferred ICP4 proteins are translated from ICP4 mRNAs (e.g., SEQ ID NO:30). The mRNA encoding ICP4 may be SEQ ID NO:30. Preferably, the ICP4 mRNA is at least 80% identical to SEQ ID NO:30 or a fragment thereof that is at least 200 nucleotides long. In some aspects, the vaccine composition of the invention, comprising at least one mRNA encoding a Herpes Simplex Virus (HSV) glycoprotein selected from the group consisting of a) an HSV glycoprotein D (gD) or an immunogenic fragment thereof having an amino acid sequence which is 70% or more identical to the amino acid sequence of SEQ ID NO:11, b) an HSV glycoprotein B (gB) or an immunogenic fragment thereof having an amino acid sequence which is 70% or more identical to the amino acid sequence of SEQ ID NO:10, and c) an HSV glycoprotein E (gE) or an immunogenic fragment thereof having an amino acid sequence which is 70% or more identical to the amino acid sequence of SEQ ID NO:4 or 80% or more identical to the amino acid sequence of SEQ ID NO:5, or any combination thereof; optionally d) an HSV ICP4 or an immunogenic fragment thereof having an amino acid sequence which is 70% or more identical to the amino acid sequence of SEQ ID NO:31, or any combination thereof. In some further aspects, the vaccine composition of the invention, comprising: (i) UL48 and gD and/or gB, optionally ICP4 (e.g., as above); (ii) UL 48 and UL49 with gE; (iii) UL11, UL16, and UL21 with gE, gD, and/or gB; or (iv) UL31 and UL34 with gD and/or gB. In particular, as discussed above some further embodiments, the immunogenic and/or vaccine composition of the invention, comprises nucleic acids encoding UL11, UL16, and UL21 as defined herein with one or more nucleic acids encoding gE, gD, and/or gB, or any fragments thereof.

The term “gE” when used herein may sometimes be referred to as “glycoprotein E”. SEQ ID NO:4 depicts exemplarily an amino acid sequence of HSV-2 gE, also deposited with NCBI GenBank under accession number AHG54732.1. However the term “gE” also encompasses gE polypeptides having an amino acid sequence which shares a certain degree of identity with the amino acid sequence shown in SEQ ID NO:4 and also encompasses polypeptides having mutations relative to the reference sequence shown in SEQ ID NO:4 as described herein. Accordingly, the term “gE” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO:4 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO:4. Preferred gE proteins translated from mRNAs of the invention can form a dimer with UL48, a trimer with UL31 and UL34 and a tetramer with UL11, UL16 and UL21.

The mRNA encoding gE may also consist of the cytoplasmic domain of HSV polypeptide gE. Preferably, the gE mRNA is at least 80% identical to SEQ ID NO:21 or a fragment thereof that is at least 200 nucleotides long. Preferably, the mRNA is at least 80% identical to SEQ ID NO:21 or a fragment thereof that is at least 200 nucleotides long

The cytoplasmic domain of gE encoded by the mRNA of the vaccine composition of the present invention preferably comprises an amino acid sequence as set forth in SEQ ID NO:5. However, it is also envisioned herein that the cytoplasmic domain of gE comprises an amino acid sequence having a sequence identity of 99%, 98%, 97%, 96%, 95%, 94%, 93%, 92%, 91%, 90%, 89%, 88%, 87%, 86%, 85%, 84%, 83%, 82%, 81%, 79%, 78%, 77%, 76%, 75% or preferably 80% or more compared to the amino acid sequence of SEQ ID NO:5 or polypeptides having up to 1, 2, 3, 4, 5, 6, 7, 8, 9, 10, 11, 12, 13, 14, 15, 16, 17, 18, 19, 20, 21, 22, 24, 25, 26, 27, or preferably 23 amino acid substitutions, insertions and/or deletions compared to the amino acid sequence of SEQ ID NO:5. Preferred cytoplasmic domains of gE translated from mRNAs of the invention can form a dimer with UL48, a trimer with UL31 and UL34 and a tetramer with UL11, UL16 and UL21.

The mRNA encoding the HSV glycoprotein gD of the vaccine composition the present invention preferably encodes an amino acid or an immunogenic fragment thereof which is 70% or more identical to the amino acid sequence of SEQ ID NO:11. Preferably, the mRNA is at least 80% identical to SEQ ID NO:22 or a fragment thereof that is at least 200 nucleotides long.

The term “gD” when used herein may sometimes be referred to as “glycoprotein D”. SEQ ID NO:11 depicts exemplarily an amino acid sequence of HSV-2 gD. However the term “gD” also encompasses gD polypeptides having an amino acid sequence which shares a certain degree of identity with the amino acid sequence shown in SEQ ID NO:11 and also encompasses polypeptides having mutations relative to the reference sequence shown in SEQ ID NO:11 as described herein. Accordingly, the term “gD” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO:11 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO:11. Preferred gD proteins translated from mRNA of the vaccine composition can form a complex with UL11, UL16 and UL21 proteins translated from mRNA of the vaccine composition. Resulting preferred gD proteins can form a dimer with UL48, a trimer with UL31 and UL34 and a tetramer with UL11, UL16 and UL21.

The mRNA encoding the HSV glycoprotein gB of the vaccine composition the present invention preferably encodes an amino acid or an immunogenic fragment thereof which is 70% or more identical to the amino acid sequence of SEQ ID NO:10. Preferably, the mRNA is at least 80% identical to SEQ ID NO:23 or a fragment thereof that is at least 200 nucleotides long.

The term “gB” when used herein may sometimes be referred to as “glycoprotein B”. SEQ ID NO:10 depicts exemplarily an amino acid sequence of HSV-2 gB. However the term “gB” also encompasses gB polypeptides having an amino acid sequence which shares a certain degree of identity with the amino acid sequence shown in SEQ ID NO:10 and also encompasses polypeptides having mutations relative to the reference sequence shown in SEQ ID NO:10 as described herein. Accordingly, the term “gB” encompasses polypeptides having an amino acid sequence identity of 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 69%, 68%, 67%, 66%, 65% or preferably 70% or more compared to the amino acid sequence of SEQ ID NO:10 or polypeptides having up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or preferably 165 amino acid substitutions, insertions and/or deletions compared to the amino acid sequence of SEQ ID NO:10. Preferred gB proteins translated from mRNA of the vaccine composition can form a complex with UL11, UL16 and UL21 proteins translated from mRNA of the vaccine composition. Resulting preferred gB proteins can form a dimer with UL48, a trimer with UL31 and UL34 and a tetramer with UL11, UL16 and UL21.

Also preferred is a nucleoside modified mRNA encoding gE, gB or gD or an immunogenic fragment thereof. Preferred fragments are described in US2020/0276300 and encompass pseudouridine residues, preferably m1ψ (1-methylpseudouridine); m1acp3ψ (1-methyl-3-(3-amino-5-carboxypropyl)pseudouridine, Wm (2′-0-methylpseudouridine), m5D (5-methyldihydrouridine), m3ψ (3-methylpseudouridine), or any combination thereof. Specifically, the mRNAs of SEQ ID NO:12 and 13, respectively, are such nucleoside modified gD and gE mRNAs, respectively. Further examples of pseudouridine-modified sequences are shown in SEQ ID NOs: 25-30.

As stated, each mRNA of the invention, may encode a protein containing mutations, such as insertions, deletions and substitutions relative to the reference sequences shown in SEQ ID NO:1 (UL11), SEQ ID NO:2 (UL16), SEQ ID NO:3 (UL21), SEQ ID NO:6 (UL48), SEQ ID NO:7 (UL49), SEQ ID NO:8 (UL31), SEQ ID NO:9 (UL34), SEQ ID NO:4 (gE), SEQ ID NO:5 (cytoplasmic domain of gE), SEQ ID NO:10 (gB) and SEQ ID NO:11 (gD).

In one aspect, the mRNA the of the invention encodes a UL48 protein alone or in combination with an mRNA encoding a glycoprotein selected from the group of gD or gB.

In a further preferred embodiment of the present invention, the mRNAs in the vaccine composition encode two or three structural polypeptides that form a multimeric complex after translation. Additionally, one or more mRNAs encoding glycoprotein gE, gB and/or gD or an immunogenic fragment thereof can be included in the vaccine composition.

Specifically, the vaccine compositions of the invention can comprise mRNA encoding UL48 and UL49, which when translated can form a complex. Alternately, the vaccine compositions of the invention can comprise mRNA encoding UL48, UL49 and glycoprotein gE, all of which can form a complex when translated.

In another embodiment, the vaccine compositions of the invention can comprise mRNA encoding UL11, UL16 and UL21, which when translated can form a complex. The vaccine compositions of the invention comprising mRNA encoding UL11, UL16 and UL21 can further comprise one or more mRNAs encoding glycoprotein gE, gB or gD.

Alternately, the vaccine compositions of the invention can comprise mRNA encoding UL31 and UL34, which when translated can form a complex. The vaccine compositions of the invention comprising mRNA encoding UL31 and UL34 can further comprise one or more mRNAs encoding glycoprotein gB or gD.

The mRNA in the vaccine compositions can encode HSV-1 polypeptides, HSV-2 polypeptides or a mixture thereof.

The vaccine composition of the invention may further comprise a pharmaceutically acceptable carrier or adjuvant.

The terms “carrier” and “excipient” are used interchangeably herein.

Pharmaceutically acceptable carriers include, but are not limited to diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO2), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), anti-foaming agents (e.g. Simethicone), thickening agents (e.g. methylcellulose or hydroxyethylcellulose), sweetening agents (e.g. sorbitol, saccharin, aspartame, acesulfame), flavouring agents (e.g. peppermint, lemon oils, butterscotch, etc), humectants (e.g. propylene, glycol, glycerol, sorbitol). Further pharmaceutically acceptable carriers are (biodegradable) liposomes; microspheres made of the biodegradable polymer poly(D,L)-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable excipients are inter alia described in Remington's Pharmaceutical Sciences, 15th Ed., Mack Publishing Co., New Jersey (1991) and Bauer et al., Pharmazeutische Technologie, 5th Ed., Govi-Verlag Frankfurt (1997). The person skilled in the art will readily be able to choose suitable pharmaceutically acceptable carriers, depending, e.g., on the formulation and administration route of the pharmaceutical composition.

The term “adjuvant” as used herein refers to a substance that enhances, augments or potentiates the host's immune response (antibody and/or cell-mediated) to an antigen or fragment thereof. Exemplary adjuvants for use in accordance with the present invention include inorganic compounds such as alum, aluminum hydroxide, aluminum phosphate, calcium phosphate hydroxide, the TLR9 agonist CpG oligodeoxynucleotide, the TLR4 agonist monophosphoryl lipid (MPL), the TLR4 agonist glucopyranosyl lipid (GLA), the water in oil emulsions Montanide ISA 51 and 720, mineral oils, such as paraffin oil, virosomes, bacterial products, such as killed bacteria Bordetella pertussis, Mycobacterium bovis, toxoids, nonbacterial organics, such as squalene, thimerosal, detergents (Quil A), cytokines, such as IL-1, IL-2, IL-10 and IL-12, and complex compositions such as Freund's complete adjuvant, and Freund's incomplete adjuvant. Generally, the adjuvant used in accordance with the present invention preferably potentiates the immune response to the multimeric complex of the invention and/or modulates it towards the desired immune responses.

The term “pharmaceutically acceptable” means a non-toxic material that does not interfere with the effectiveness of the biological activity of the immunogenic composition according to the present invention.

Use of the Vaccine Composition

As demonstrated in the appended Examples, the present inventors have found that the immunogenic composition and/or the vaccine composition as disclosed herein is advantageous as a medicament. Thus, the present invention also pertains to the use of the vaccine composition in a method of inducing an immune response against HSV in a subject. In particular, the invention also pertains to the use of the vaccine composition comprising the immunogenic composition which comprises at least one, such as at least two, such as all three nucleic acid(s) as defined in (i), (ii) and (iii) as disclosed herein in a method of inducing an immune response against HSV-2 in a subject. As demonstrated in appended Examples 12-19, the administration of a vaccine composition comprising at least one, such as at least two, such as all three nucleic acid(s) as defined in (i), (ii) and (iii) according to the present invention was well tolerated and induced significant IgG responses after prime and boost vaccination in an animal model system. Importantly, the vaccine candidate showed surprisingly beneficial effects in terms of T cell response after immunization. Based on the extraordinary strong T cell responses, the present inventors consider that said vaccine composition will be superior to prior art vaccine compositions. It is known that T cells are crucial for the immunologic control of HSV-2 reactivation, and play several key roles in the immune response against the virus. CD4+ T cells are critical for the activation of B cells and antibody class-switching, as well as for “licensing” DCs to activate CD8+ T cells. CD8+ T cells secrete IFN-γ which performs a number of antiviral roles including as discussed in more detail in the Example section and leads to limitation of HSV viral replication, restoration of HSV-induced MHC class and chemokine production, which results in the recruitment of CD8+ T cells to the site of infection, where the cytotoxic CD8+ T cells kill cells infected by the virus.

The present inventors found that the administration of the vaccine composition as disclosed herein induced very potent induction of IFN-γ secretion in subjects, which was significantly higher than responses elicited by corresponding protein vaccination, as well as lead to higher IL-2 and TNF-α secretion. Importantly, administration of vaccine composition as disclosed herein elicited significantly more multifunctional T cells that produced more than one cytokine: IFN-γ/IL-2; IFN-γ/TNF-α and IFN-γ/IL-2/TNF-α. As multifunctional HSV-specific T cells have been implicated as an important factor for immunologic control of herpes virus infections, the present inventors envision this vaccine compositions will not only be useful for protection from viral infection, but will also be useful in the therapeutic and/or preventive treatment of reoccurring reactivation of the latent virus.

Thus, herein is provided a method for therapeutic treatment and/or prophylactic treatment of an HSV-2 infection comprising administration of the immunogenic composition as defined herein or the vaccine composition as defined herein to a subject, such as a human subject, in need thereof. A subject in need thereof, may be a subject susceptible to HSV-2 infection or a subject already infected with HSV-2. In particular, said administration is administration of a therapeutically effective amount of said immunogenic composition or vaccine composition. In one embodiment, said effective amount of said immunogenic composition or said vaccine composition is an amount which elicits an immune response resulting in therapeutic treatment and/or prophylactic treatment of HSV-2, for example resulting in ameliorating and/or eliminating symptoms associated with said HSV-2 infection or preventing symptoms associated with said HSV-2 infection. In one embodiment, said immunogenic composition or vaccine composition comprises at least one, such as at least two, such as all three nucleic acid(s) as defined in (i), (ii) and (iii) as disclosed herein. In one embodiment, said immunogenic composition or vaccine composition comprises all three nucleic acids as defined in (i), (ii) and (iii) as disclosed herein.

In a preferred embodiment of the present invention the vaccine composition is used for the treatment, prevention or amelioration of HSV infection or preventing reactivation of HSV. Thus, in one embodiment there is provided a method for therapeutic treatment and/or prophylactic treatment as disclosed herein, wherein said therapeutic treatment comprises ameliorating and/or eliminating symptoms associated with said HSV-2 infection. In another embodiment, said method for therapeutic treatment and/or prophylactic treatment is a method for prophylactic treatment and comprises preventing symptoms associated with said HSV-2 infection.

HSV infection may be selected from the group consisting of an HSV-1 infection, an HSV-2 infection, a primary HSV infection, a flare, recurrence, or HSV labialis following a primary HSV infection, a reactivation of a latent HSV infection, an HSV encephalitis, an HSV neonatal infection, a genital HSV infection, and an oral HSV infection.

In one embodiment, said HSV-2 infection is selected from a group consisting of a primary HSV-2 infection and a reactivation of a latent HSV-2 infection, such as wherein said HSV-2 infection is a reactivation of a latent HSV-2 infection. In one embodiment, said reactivation of a latent HSV-2 infection is a flare, a recurrence and/or an HSV-2 labialis following a primary HSV-2 infection. In one particular embodiment, said HSV-2 infection is a genital HSV-2 infection and/or an oral HSV-2 infection.

Accordingly, the vaccine composition may be used in fighting diseases caused by HSV and/or related symptoms. Accordingly, the present method may be used in fighting diseases caused by HSV and/or related symptoms, for example encephalitis caused by HSV-2 infection. Thus, in one embodiment, said therapeutic treatment and/or prophylactic treatment may be ameliorating and/or eliminating symptoms associated with HSV-2 encephalitis or prevention of symptoms associated with HSV-2 encephalitis. In one embodiment said therapeutic treatment and/or prophylactic treatment comprises preventing the development of HSV-2 encephalitis in said subject.

In one particular embodiment, said HSV-2 infection is a neonatal infection. In another embodiment, said prophylactic treatment comprises preventing a reactivation of a latent HSV-2 infection, such as preventing a flare, a recurrence and/or an HSV-2 labialis following a primary HSV-2 infection. In yet another particular embodiment, said prophylactic treatment comprises preventing the development of HSV-2 encephalitis in said subject. It is also envisaged that the vaccine composition of the present invention may be used for clearing the virus in a subject, i.e. after treatment no HSV can be detected in a suitable sample obtained from the subject using suitable methods known to those of ordinary skill in the art, e.g. PCR, ELISA etc. Thus, the vaccine composition of the present invention may be used to block primary infection, stop primary disease, block virus reactivation and re-infection, and to block latency.

To reduce the risk of genital herpes, a prophylactic vaccine to prevent the first HSV infection of the mother is desirable, whereas an effective therapy is needed in the case a mother is diagnosed with an active HSV infection. This is important in order to prevent neonatal HSV-2 infection. The present invention may be applied as a prophylactic vaccine, e.g. for expectant mothers or children, or as a therapeutic vaccine in seropositive women to prevent subclinical reactivation at the time of delivery.

In a further preferred embodiment of the present invention the vaccine composition is used in a method for inducing an immune response against HSV-1 or HSV-2 in a subject.

It is envisioned that the above mentioned therapeutically effective dose is in the range of about 10 to 250 μg total amount of nucleic acids per administration. Thus in one embodiment, said method comprises administration of the immunogenic composition or the vaccine composition in a single dose to said subject, wherein said single dose corresponds to the total amount of nucleic acids as defined in i), ii) and iii) and is less than about 200 μg, such as less than about 150 μg, such as less than about 100 μg, such as is between about 10 μg and about 100 μg. In the case wherein the priming dose alone elicits sufficient protective levels of immunity, it is envisioned that a single administration is sufficient in order to achieve the therapeutic and/or prophylactic effect. In other cases, it is envisioned that a more than one dose is required to elicits sufficient protective levels of immunity, the method may comprise multiple administrations, such as a priming dose and at least one boosting dose. In one embodiment, said method comprises multiple administration of the immunogenic composition or the vaccine composition to said subject. In one embodiment, said multiple administration comprises a prime vaccination and at least one boost vaccination, such as wherein said multiple administration is a two-dose prime-boost regimen comprising said prime vaccination and one of said at least one boost vaccination. In a particular embodiment, said two-dose prime-boost regimen is administered repeatedly on an annual basis to said subject. In a particular embodiment, said two-dose prime-boost regimen is administered within a year from the administration of the prime dose and only the boost dose is administered repeatedly on an annual basis to said subject. In one embodiment, a boost dose is adminstered on annual or semi annual basis to said subject. In one embodiment, a prime dose and optionally a first boost dose are administered year 1, a second boost dose is adminstered year 2, and subsequent doses are administered annually 1, 2 or 3 years after administration of the second boost dose. For example, said boost doses can be adminstered year 2, 3, 4, 5 etc; or year 2, 4, 6, 8 etc; or year 3, 6, 9, 12 etc; or year 2, 3, 5, 8; or according to any other regimen. In one embodiment, the dose of said prime vaccination corresponds to the total amount of nucleic acids as defined in i), ii) and iii) and is less than about 200 μg, such as less than about 150 μg, such as less than about 100 μg, such as is between about 10 μg and about 100 μg. In one embodiment, the dose of said at least one boost vaccination corresponds to the total amount of nucleic acids as defined in i), ii) and iii) and is less than about 200 μg, such as less than about 150 μg, such as less than about 100 μg, such as is between about 10 μg and about 100 μg.

In one particular embodiment, the time interval between said prime vaccination and said at least one boost vaccination is less than about 10 weeks, such as less than about 9 weeks, such as less than about 8 weeks, such as a time interval between about 2 weeks and about 8 weeks. The terms “polynucleotide”, “nucleotide sequence”, “nucleic acid” or “nucleic acid molecule” are used interchangeably herein and refer to a polymeric form of nucleotides which are usually linked from one deoxyribose or ribose to another. The term “polynucleotide” preferably includes single and double stranded forms of DNA or RNA. A nucleic acid molecule of this invention may include both sense and antisense strands of RNA (containing ribonucleotides), cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The vaccine composition of the invention may be used in a prime boost regimen. In the prime boost regimen, a prime/boost vaccine is used which is composed of two or more types of vaccine including a vaccine used in primary immunization (prime or priming) and a vaccine used in booster immunization (boost or boosting). The vaccine used in primary immunization and the vaccine used in booster immunization may differ from each other or may be the same. Primary immunization and boosting immunization may be performed sequentially, this is, however, not mandatory. The prime/boost regimen includes, without limitation, e.g. mRNA prime/protein boost. However, the boosting composition can also be used as priming composition and said priming composition is used as boosting composition.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

The term “about” or “approximately” as used herein means within 20%, preferably within 10%, and more preferably within 5% of a given value or range. It includes also the concrete number, e.g., about 20 includes 20.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol I 1976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

### LIST OF REFERENCES

Truong N R, Smith J B, Sandgren K J, Cunningham A L. Mechanisms of Immune Control of Mucosal HSV Infection: A Guide to Rational Vaccine Design. Front Immunol. 2019 Mar. 6; 10:373.
Egan K P, Wu S, Wigdahl B, Jennings S R. Immunological control of herpes simplex virus infections. J Neurovirol. 2013 August; 19(4):328-45.
Khan A A, Srivastava R, Chentoufi A A, Kritzer E, Chilukuri S, Garg S, Yu D C, Vahed H, Huang L, Syed S A, Furness J N, Tran T T, Anthony N B, McLaren C E, Sidney J, Sette A, Noelle R J, BenMohamed L. Bolstering the Number and Function of HSV-1-Specific CD8+ Effector Memory T Cells and Tissue-Resident Memory T Cells in Latently Infected Trigeminal Ganglia Reduces Recurrent Ocular Herpes Infection and Disease. J Immunol. 2017 Jul. 1; 199(1):186-203.
Kuo T, Wang C, Badakhshan T, Chilukuri S, BenMohamed L. The challenges and opportunities for the development of a T-cell epitope-based herpes simplex vaccine. Vaccine. 2014 Nov. 28; 32(50):6733-45.
Ruchi Srivastava, Pierre-Gregoire Coulon, Soumyabrata Roy, Sravya Chilukuri, Sumit Garg, Lbachir BenMohamed; Phenotypic and Functional Signatures of Herpes Simplex Virus-Specific Effector Memory CD73+CD45RAhighCCR7lowCD8+ TEMRA and CD73+CD45RAlowCCR7lowCD8+ TEM Cells Are Associated with Asymptomatic Ocular Herpes. J Immunol 15 Oct. 2018; 201 (8): 2315-2330.
Presnyak V, Alhusaini N, Chen Y H, Martin S, Morris N, Kline N, Olson S, Weinberg D, Baker K E, Graveley B R, Coller J. Codon optimality is a major determinant of mRNA stability. Cell. 2015 Mar. 12; 160(6):1111-24.
Hou X, Zaks T, Langer R, Dong Y. Lipid nanoparticles for mRNA delivery. Nat Rev Mater. 2021; 6(12):1078-1094.
Kowalski P S, Rudra A, Miao L, Anderson D G. Delivering the Messenger: Advances in Technologies for Therapeutic mRNA Delivery. Mol Ther. 2019 Apr. 10; 27(4):710-728.
Kimura T, Leal J M, Simpson A, Warner N L, Berube B J, Archer J F, Park S, Kurtz R, Hinkley T, Nicholes K, Sharma S, Duthie M S, Berglund P, Reed S G, Khandhar A P, Erasmus J H. A localizing nanocarrier formulation enables multi-target immune responses to multivalent replicating RNA with limited systemic inflammation. Mol Ther. 2023 Aug. 2; 31(8):2360-2375.
P Pardi N, Hogan M J, Porter F W, Weissman D. mRNA vaccines—a new era in vaccinology. Nat Rev Drug Discov. 2018 April; 17(4):261-279.

### EXAMPLES

The following hypothetical Examples illustrate the invention, but are not to be construed as limiting the scope of the invention.

### Example 1

PBMC from four HSV-2-infected individuals and two uninfected individuals were thawed and left rest overnight. Cells were seeded onto plates at 5×105 cells/well and subsequently stimulated with 5 μg/mL of HSV-2 UL48 mRNA alone or with 5 μg/mL UL49 mRNA for 48 h. Supernatants were thereafter collected and analyzed for the secretion of IFN-γ with a Luminex instrument. The background signal (generated from buffer-stimulated cells) was subtracted from each well and results were expressed as pg/ml.

### Example 2

Splenocytes from HSV-2 infected and control guinea pigs (1×105 cells) were mixed with 10 μg/mL of HSV-2 UL31 mRNA and 10 μg/mL UL34 mRNA. Cells were then transferred onto ELISPOT anti-interferon gamma (IFN-γ) antibody-coated plates (Multiscreen HTS Plates; Millipore) and incubated for 20 h. Plates were thereafter developed according to standard ELISPOT protocols and the IFN-γ secreting cells were quantified as spots using an automated reader. Unstimulated cells and 20 μg/mL of PHA were used as negative and positive controls, respectively.

### Example 3

PBMC from fourteen HSV-2-infected and six uninfected individuals were thawed and left rest overnight. Cells were plated onto ELISPOT anti-interferon gamma (IFN-γ) antibody coated plates at 2×105 cells/well. Cells were subsequently stimulated with 5 μg/mL of HSV-2 UL31 mRNA and 5 μg/mL of HSV-2 UL34 mRNA for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×105 PBMC.

### Example 4

PBMC from four HSV-2-infected and two uninfected individuals were thawed and left rest overnight. Cells were plated onto ELISPOT anti-interferon gamma (IFN-γ) antibody coated plates at 2×105 cells/well. Cells were subsequently stimulated with 5 μg/mL of HSV-2 UL48 mRNA alone or with 5 μg/mL UL49 mRNA, for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×105 PBMC.

### Example 5

PBMC from fourteen HSV-2-infected and six uninfected individuals were thawed and left rest overnight. Cells were plated onto ELISPOT anti-interferon gamma (IFN-γ) antibody coated plates at 2×105 cells/well. Cells were subsequently stimulated with 5 μg/mL of HSV-2 UL48 mRNA alone or in combination with 5 μg/mL UL49 mRNA for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×105 PBMC.

### Example 6

PBMC from four HSV-2-infected and two uninfected individuals were thawed and left rest overnight. Cells were plated onto ELISPOT anti-interferon gamma (IFN-γ) antibody coated plates at 2×105 cells/well. Cells were subsequently stimulated with 5 μg/mL of HSV-2 UL11 mRNA, 5 μg/mL UL16 mRNA and 5 μg/mL UL21 mRNA, or the respective mRNA encoding UL11, UL16 or UL21 normalized to the amount of the single proteins in the combination, for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×105 PBMC.

### Example 7

PBMC from fourteen HSV-2-infected and six uninfected individuals were thawed and left rest overnight. Cells were plated onto ELISPOT anti-interferon gamma (IFN-γ) antibody coated plates at 2×105 cells/well. Cells were subsequently stimulated with 5 μg/mL of HSV-2 UL11 mRNA, 5 μg/mL UL16 mRNA and 5 μg/mL UL21 mRNA for 48 h. Plates were thereafter developed according to manufacturer's instructions and the IFN-γ secreting cells were counted as spots with an automated reader. The background signal (generated from buffer-stimulated cells) was subtracted from each well and results were expressed as SFU (spot forming units) per 2×105 PBMC.

### Example 8

PBMC from four HSV-2-infected individuals and two uninfected individuals were thawed and left rest overnight. Cells were seeded onto plates at 5×105 cells/well and subsequently stimulated with 5 μg/mL of HSV-2 UL31 mRNA and 5 μg/mL of HSV-2 UL34 mRNA for 48 h. Supernatants were thereafter collected and analyzed for the secretion of IFN-γ with a Luminex instrument. The background signal (generated from buffer-stimulated cells) was subtracted from each well and results were expressed as pg/ml.

### Example 9

PBMC from four HSV-2-infected individuals and two uninfected individuals were thawed and left rest overnight. Cells were seeded onto plates at 5×105 cells/well and subsequently stimulated with 5 μg/mL of HSV-2 UL11 mRNA, 5 μg/mL UL16 mRNA and 5 μg/mL UL21 mRNA for 48 h. Supernatants were thereafter collected and analyzed for the secretion of IFN-γ with a Luminex instrument. The background signal (generated from buffer-stimulated cells) was subtracted from each well and results were expressed as pg/ml.

### Example 10: mRNA Transfection and Western Blot

Methods:

HEK 293T cells were seeded at a concentration of 0.4×106/ml in 12 well plates containing RPMI media and 10% FBS, and incubated at 37° C. and 5% CO2. The next day the cells were transfected using Invitrogen Lipofectamine MessengerMAX Transfection kit. 2-4.5 μl of 1 μg/μl mRNA (SEQ ID NO:25 and SEQ ID NOs: 26, 27 and 28) was added per well. The empty transfection wells had only the transfection reagent added, nothing was added to the negative control wells.

The cells were harvested over the following days. To do this, the media was removed from the wells and 70 μl of chilled Thermo Scientific RIPA Lysis and Extraction Buffer, along with 10 μl 7× complete, EDTA-free Protease Inhibitor Cocktail was added to each well. The plate was incubated at 4° C. for 2-3 minutes, and the cells were then detached using a cell scraper. The cell-buffer mix was transferred to a 1.5 ml Eppendorf tube and incubated on ice. 70 μl of 2× Biorad Laemli buffer containing 50 mM DTT was added to each tube, and the samples were boiled at 90° C. for 5 minutes. For the positive controls, a sample of the recombinant protein(s) was added to RIPA buffer and proteinase and treated in an identical way to the other samples. Prior to loading the samples on a gel, 1 μl of Thermo Scientific Pierce Universal Nuclease for cell lysis was added to each. 20 μl of each sample was then loaded onto an Invitrogen Bolt 4-12% Bis-Tris Plus gel, which was run at 90V for 40 minutes. Following this, the samples were transferred using an iBlot 2 system and iBlot 2 mini PVDF Transfer Stacks. The settings used were: 20V for 1 minute, 23V at 4 minutes, and 25V for 90 seconds.

The membrane was then blocked overnight at 4° C. in 5% BSA TBS containing 0.1% Tween-20. Primary antibodies, which were either purchased commercially or produced in-house, were added to the blocking buffer at a 1:1000 concentration and incubated at room temperature while being gently shaken for 1 hour. The membrane was then washed 3× with TBS containing 0.1% Tween-20. Following this, the secondary antibody was added in a 1:5000 concentration in blocking buffer and incubated at room temperature while being gently shaken for 1 hour. The membrane was again washed 3× with BST containing 0.1% Tween-20.

To perform the imaging, 300 μl of SuperSignal West Femto Maximum Sensitivity Substrate was applied to the membrane and it was imaged using a Full frame camera with a 100 mm F/2.8 lens and dark box.

Results:

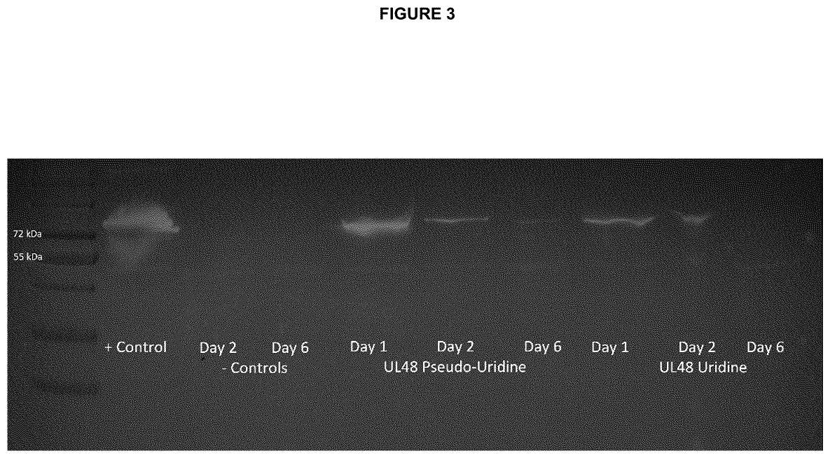

FIG. 3 (Western Blot of the protein derived from the mRNA UL48—SEQ ID NO:25) clearly shows expression of UL48 protein following transfection of the mRNA construct. Highest expression is apparent 1 day after transfection, and then falls with time. This indicates robust protein expression as a result of the application of our UL48 mRNA constructs (e.g., SEQ ID NO:25).

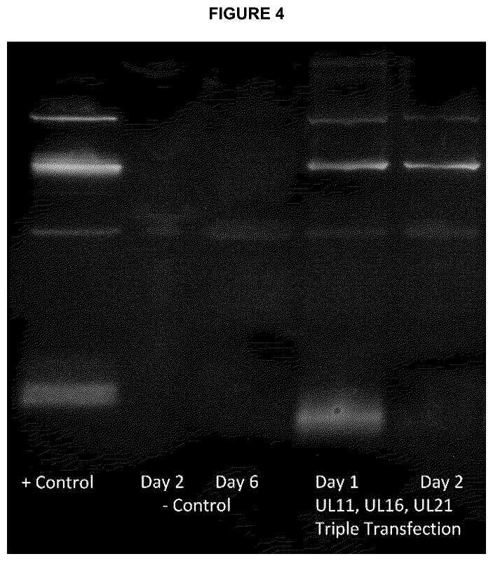

FIG. 4 (Western Blot of the protein derived from the mRNAs UL11, UL16 and UL21—SEQ ID NOs: 26, 27 and 28) clearly shows expression of UL11, UL16, and UL21 following co-transfection of our mRNA constructs. As observed for UL48, expression is highest one day post-transfection.

### Example 11: Immunogenicity Data Using PBMCs

Methods:

PBMC Propagation and IFNγ ELISA Protocol

PBMCs collected from HSV-2+donors were thawed and grown overnight in RPMI containing 10% FBS in 12 well plates at a concentration of 1×106 cells/ml. The next day the cells were transfected using Invitrogen Lipofectamine MessengerMAX Transfection kit. 1-2 μl of 1 μg/μl mRNA (SEQ ID NO:25, SEQ ID NO:2 and SEQ ID NO:30) was added per well. The empty transfection wells had only the transfection reagent added, nothing was added to the negative control wells.

The samples were harvested 3 days post-transfection. The supernatant was centrifuged at 500RCF for 6 minutes. Afterwards the IFNγ levels were assessed using an Invitrogen Human IFN Gamma Uncoated ELISA kit and F96 Maxisorp Nunc-Immuno plates. OD450 measurements were performed in a Tecan Infinite M Plex plate reader.

Results:

The ELISA results show the secretion of IFNγ in PBMCs from HSV 2+donors triggered by the pseudouridine UL48, gD and ICP4 mRNAs. These data indicate that specific immune responses are triggered by the expression of the applied HSV-2 mRNAs. No mRNA or transfection reagent was added to the negative control wells. For the blank wells no biological sample was added during the ELISA.

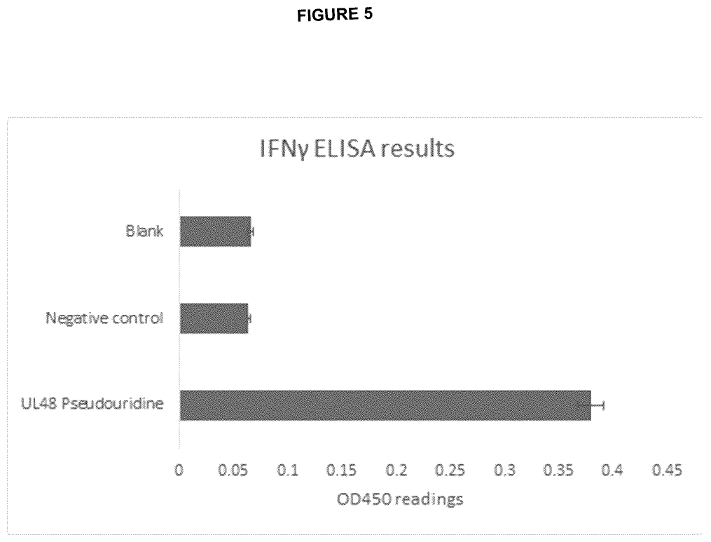

FIG. 5 shows a measurement of IFNγ release using ELISA upon incubation with the UL48 modified mRNA, no mRNA or transfection reagent was added to the negative control wells, for the blank wells no biological sample was added during the ELISA.

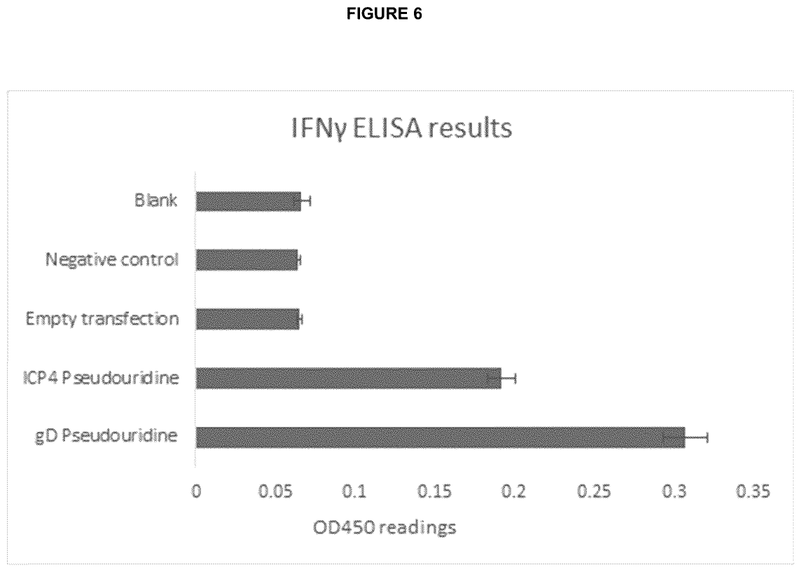

FIG. 6 shows a measurement of IFNγ release using ELISA upon incubation with the ICP4 and gD modified mRNAs, the empty transfection wells had only the transfection reagent added, nothing was added to the negative control wells. For the blank wells no biological sample was added during the ELISA.

### Conclusion

The Western blot and PBMCs experiments were performed to assess the stability and functionality of the present mRNA constructs. The design of mRNAs to be used in a vaccine composition is crucial and therefore has been evaluated. With regards to stability, the present vaccine mRNAs comprise an optimized 5′ cap, 5′ and 3′ UTRs, and polyA tail. The Western blot analyses (FIGS. 3 and 4) clearly show a robust expression of the UL48, UL11, UL16, and UL21 mRNAs and indicate that the constructs are stable, which is fundamental for the efficacy of a vaccine comprised of said mRNAs.

In addition to the stability, it has also been confirmed that the immune responses can be triggered by the vaccine components. In that context, the vaccine mRNAs were optimized using modified residues with 1-methyl-pseudouridine to reduce the innate non-specific immune responses. The IFNγ ELISA results indeed indicate the specific release of this immune factor upon the incubation with the functional UL48, gD and ICP4 mRNAs (FIGS. 5 and 6).

### Examples 12-20

In the present Examples 12-20 the immunogenicity of mRNA HSV-2 vaccine candidate RBT26, as defined herein, is assessed. In particular, the present examples surprisingly show that the mRNA vaccine candidate is much more potent than the corresponding protein vaccine. mRNA HSV-2 vaccine candidate RBT26 comprises the mRNAs comprising nucleotide sequences SEQ ID NO:86, 87 and 88 which encode the polypeptides RBT26.1 (SEQ ID NO:1), RBT26.2 (SEQ ID NO:2) and RBT26.3 (SEQ ID NO:3), respectively.

For the sake of clarity, herein the term “mRNA RBT26 vaccine” or “mRNA RBT26 vaccine candidate” refers to a composition comprising nucleotide sequences encoding the three polypeptides RBT26.1 (SEQ ID NO:1), RBT26.2 (SEQ ID NO:2) and RBT26.3 (SEQ ID NO:3). As uses herein, said three polypeptides are also referred to as UL11, UL16 and UL21, respectively. Similarly the term “protein RBT26 vaccine” or “protein RBT26 vaccine candidate” refers to a composition comprising three polypeptides RBT26.1 (SEQ ID NO:1), RBT26.2 (SEQ ID NO:2) and RBT26.3 (SEQ ID NO:3).

Importantly, while both mRNA and protein RBT26 vaccination elicited antigen-specific T cells and were well tolerated, the mRNA vaccination induced very potent induction of IFN-γ secretion, which was significantly higher than responses elicited by protein vaccination. In addition, mRNA vaccination stimulated significantly more T cells to secrete IL-2 and TNF-α than the protein vaccine. It was surprisingly found that mRNA vaccination elicited significantly more multifunctional T cells that produce more than one cytokine: IFN-γ/IL-2; IFN-γ/TNF-α and IFN-γ/IL-2/TNF-α. Such polyfunctional HSV-specific T cells have been implicated as an important factor for immunologic control of herpes virus infections. Furthermore, mRNA vaccination elicited antigen-specific CD4+ and CD8+ T cells, as determined by intracellular cytokine staining for IFN-γ production. The CD8+ T cell responses were very strong consisting of up to 14% of the total pool of CD8+ T cells in the spleen in some animals.

### Example 12

In this Example, the production of the mRNAs used in the present examples is described. mRNA RBT26.1 encodes protein RBT26.1 (SEQ ID NO:1). Example RNA sequences encoding said protein are SEQ ID NO:16 and 32. SEQ ID NO:32 is a codon-optimized variant of SEQ ID NO:16.

In addition SEQ ID NO:86 is a nucleoside modified variant of SEQ ID NO:32. The present study utilized RNA sequence according to SEQ ID NO:26, which comprises SEQ NO ID:86 and, untranslated regions (UTRs) and a polyA-tail. mRNA RBT26.2 encodes protein RBT26.2 (SEQ ID NO:2). Example RNA sequences encoding said protein are SEQ ID NO:17 and 33. SEQ ID NO:33 is a codon-optimized variant of SEQ ID NO:17.

In addition SEQ ID NO:87 is a nucleoside modified variant of SEQ ID NO:33. The present study utilized RNA sequence according to SEQ ID NO:27, which comprises SEQ NO ID:87, untranslated regions (UTRs) and a polyA-tail. mRNA RBT26.3 encodes protein RBT26.3 (SEQ ID NO:3). Example RNA sequences encoding said protein are SEQ ID NO:18 and 34. SEQ ID NO:34 is a codon-optimized variant of SEQ ID NO:18.

In addition SEQ ID NO:88 is a nucleoside modified variant of SEQ ID NO:34. The present study utilized RNA sequence according to SEQ ID NO:28, which comprises SEQ NO ID:88, untranslated regions (UTRs) and a polyA-tail.

Material and Method

RBT26.1, RBT26.2 and RBT26.3 mRNAs were prepared from linear DNA templates generated by PCR from plasmids encoding HSV-2 UL11, UL16 and UL21 coding sequences and 5′ and 3′ UTRs, respectively. To obtain mRNAs with modified nucleosides, the transcription reaction was assembled with the replacement of uridine nucleotide triphosphate with the triphosphate derivative 1-methylpseudouridine (m1ψ). Custom made primers (TWIST bioscience, USA) encoding the T7 promoter region, and parts of the 5′ and 3′ UTRs, and the polyA sequences were used for the PCR amplification. The primers used were manufactured by TWIST bioscience and had the following sequences:

(SEQ ID NO: 56)
Forward primer:
5′ TAA TAC GAC TCA CTA TAA GG ACT CAC TAT
TTG TTT TCG CGC CCA GTT GCA AAA AGT GTC
ACC ATG 3′
Reverse primer:
(SEQ ID NO: 57)
5′ TT TTT TTT TTT TTT TTT TTT TTT TTT TTT
TTT TTT TTT TTT TTT TTT TTT TTT TTT TTT
TTT TTT TTT TTT TTT TTT TTT TTT TTT GTA
ATC CAG AGG TTG ATT G 3′

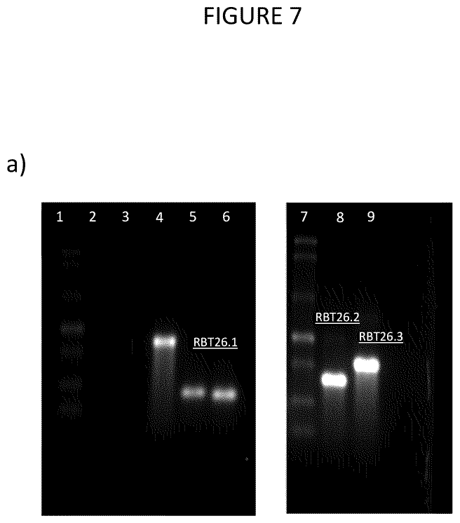

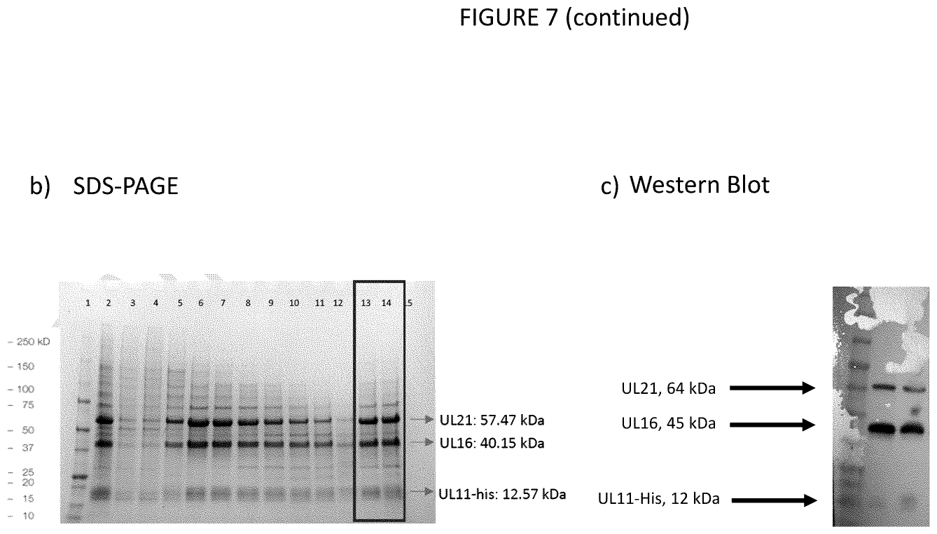

DNA templates were amplified on a thermocycler using the primers described above for 35 cycles and an annealing temperature of 60° C. PCR products were verified on agarose gel electrophoresis and used as templates for the in vitro transcription (IVT) reaction using the HiScribe T7 mRNA Kit with CleanCap Reagent AG (Cat no NEB-E2080S, New England Biolab, BioNordika, Sweden) according to the manufacturer's instructions. At the end of the IVT reaction, DNase I was added to digest template DNA. Then, mRNAs were purified using LiCl precipitation according to the manufacturer's instructions and stored at −80° C. until analysis. The mRNAs RBT26.1, RBT26.2 and RBT26.3 (SEQ ID NOs: 26, 27 and 28) were analyzed on non-denaturing agarose gel electrophoresis according to standard protocol and the gel images are shown in FIG. 7a.

### Results and Conclusion

TABLE 1
Summary of specification for the individual mRNA compoments.

| Nucleotide | Technical specifications | Concentration |
|---|---|---|
| mRNA RBT26.1 | RNA (Factor: 40); | 1022.0 ng/μl |
| (SEQ ID NO: 26) | A260 (10 mm): N/A; | |
| | A260/280: N/A; | |
| mRNA RBT26.2 | RNA (Factor: 40); | 1025.3 ng/μl |
| (SEQ ID NO: 27) | A260 (10 mm): 25.632; | |
| | A260/280: 2.02; | |
| mRNA RBT26.3 | RNA (Factor: 40); | 1022.9 ng/μl |
| (SEQ ID NO: 28) | A260 (10 mm): 25.573; | |
| | A260/280: 2.02; | |

Obtained mRNAs were considered pure based on the A 260/280 ratio of approximately 2. mRNA aliquots were resuspended in Milli-Q Water (Milli-Q® Type 1 Ultrapure Water Systems, Merck-Millipore, Sweden). The three mRNAs were mixed according to Table 2 below and aliquots were stored at −80° C. for subsequent use.

TABLE 2
Summary of mRNAs according to the present disclosure. Ratios were
calculated according to the estimated molecular weight (MW).

| | | | Volume of each |
|---|---|---|---|
| | mRNA MW | mRNAs ratio | mRNA (μl) |
| mRNA RBT26.1 | 188.32 | 0.15 | 181 |
| (SEQ ID NO: 26) | | | |
| mRNA RBT26.2 | 454.09 | 0.36 | 435 |
| (SEQ ID NO: 27) | | | |
| mRNA RBT26.3 | 609.22 | 0.49 | 584 |
| (SEQ ID NO: 28) | | | |

Total amount of mRNA: 1200 μg

### Example 13

In this Example, the formulation of the mRNA vaccine is described.

LNPs were obtained from Precision Nanosystems, Vancouver, Canada, and mRNAs were formulated at their site. Briefly, a GenVoy ILM LNP composition using NanoAssemblr Ignite for intramuscular delivery of mRNA RBT26 vaccine candidate as prepared. One LNP formulation was prepared at the scale of 0.9 mg formulated mRNA at target concentration of 0.4 mg/mL. Ultra-centrifugation with Amicon device was used for downstream process. Final formulation was analyzed for size, PDI, total RNA concentration, encapsulation efficiency (% EE), and zeta potential. Stability of LNP formulation was evaluated after one freeze/thaw cycle. The final formulation was then aliquoted for in vivo studies, and stored at −80° C.

Material and Method

GenVoy-ILM lipid mix (cat. no. NWW0042, Precision Nanosystems, Canada) was used for preparation of the mRNA LNP formulation. 25 mM lipid mix was heated at 55° C. to fully dissolve (5-10 min) and cooled down to RT prior to use. The three mRNAs (RBT26.1, RBT26.2, and RBT26.3) were mixed (referred to as tri-mix RNA) to prepare total amount of 995 μg at final concentration of 1 mg/mL. Next, aqueous phase of the tri-mix RNA was prepared by dilution RNase free water (cat. no. 02-0201-0500, VWR, Canada) and PNI formulation buffer (pH 4.0) (lot. No. AH29918235, Cytiva, USA) to obtain an RNA stock solution having a concentration of 0.0927 mg/mL. Concentration was measured using the Nano Drop system (model AZY1915880 ThermoFisher, Canada).

mRNA LNP preparation was performed using the Ignite system (NanoAssmblr Ignite model BZ-1.0-006 Precision Nanosystems Inc, Canada). Table 3 summarizes the relevant parametres.

TABLE 3
NanoAssemblr parameters used for preparation of RNA LNPs on Ignite.

| | |
|---|---|
| Instrument | Ignite |
| Cartridge | NxGen Cartridge |
| Heating Block | N/A |
| Number of Runs | 1 |
| Formulation volume | 13 mL |
| N/P | 6 |
| Organic stream | 3.25 mL (+0.25 mL extra) 10 mM GenVoy ILM |
| Aqueous stream | 9.75 mL (+0.25 mL extra) 0.0927 mg/mL tri-mix mRNA in PNI |
| | formulation buffer, pH 4 |
| Dilution stream | 26 mL 1x PBS |
| Flow rate ratio (Aqu:Org) | 3:1 |
| Total flow rate | 12 mL/min |
| Dilution ratio | 2 (Dilution stream):1 (Micromixed particles) |
| Start waste | 1.65 mL |
| End waste | 0.05 mL |
| Total postchip volume | 13 mL micromixer + 26 mL dilution − 1.70 waste = 37.30 mL |
| Syringe size | Organic line: 5 mL |
| | Aqueous line: 10 mL |
| | Dilution line: 30 mL |

Next the LNPs were processed using centrifugal filtration. Briefly, the LNP formulation volume of 37.3 mL was diluted with of PBS to (5× bulk dilution) to reach the final volume of 186.5 mL and transferred to eight 30 kDa Amicon tubes (15 mL) (cat. no. UFC903096, Millipore Sigma, Canada). This bulk diluted formulation was centrifuged at 2500×g 4° C. for 30 mins. The centrifugation process was repeated until all the bulk diluted formulation were concentrated to total remaining volume of approximately 8 mL (approximately 1 ml in each Amicon). The formulations were diluted 5× by adding 4 mL CB1 (lot no. PRC-CB1-05, Precision Nanosystems, Canada) per Amicon tube and centrifuged again. The centrifugation-dilution process for was repeated until a 22× concentration was achieved corresponding until the total remaining volume in Amicon tubes was approximately 2.1 mL.

Next, the LNP formulations were sterile filtered manually through 0.2 μm 13 mm EZflow disc syringe filters 371-2115-OEM (cat. no. 76018-866, VWR, Canada) in a biosafety cabinet into a sterile vial. The concentration of mRNA in the formulation was confirmed after filtration using Quant-iT™ RiboGreen™ RNA Reagent kit (cat no. R11491, ThermoFisher, Canada) according to manufacturer's recommendations and subsequently adjusted in sterile CB1 buffer to three target concentrations of 0.300 mg/mL, 0.100 mg/mL and 0.040 mg/mL. The final products were aliquoted and transferred to manufacturing −80° C. freezer.

Particle size, polydispersity index (PDI) and zeta potential measurements was carried out using Dynamic Light Scattering (DLS) on Zetasizer Nano-ZS or Ultra (Malvern Instruments, UK) according to manufacturer's instructions. For this purpose, 5-20 μL of a sample was suspended in 280-295 μL of PBS buffer. For zeta potential analyzes 5-20 μL of LNP was diluted to 1000 μL with 0.1×PBS, pH 7.4.

mRNA encapsulation efficiency (EE %) measurements were performed using the fluorescence-based RiboGreen 96-well plate assay (Quant-iT™ RiboGreen™ RNA Reagent kit (cat no. R11491, ThermoFisher, Canada)) and total mRNA concentration of mRNA LNPs formulated was measured on the NanoAssemblr Ignite™ instrument. Briefly, 1×TE buffer (cat. no. T11493, ThermoFisher, Canada) was used to dilute the Quant-iT™ RiboGreen® reagent 100-fold. LNPs samples were diluted in 1×TE buffer. mRNA encapsulation efficiency will be determined by measuring fluorescence in the absence and presence of 2% Triton X-100 (cat. no. T9284-100 mL, Sigma-Aldrich, Canada) required to break the LNPs. A standard curve was prepared using provided mRNA and was used to estimate the total mRNA concentration. All readings and measurements were performed using a BioTek Synergi-H1 Hybrid multi-mode reader (Agilent, Canada) at an excitation wavelength of 485 nm and an emission wavelength of 528 nm.

EE % was calculated using the following formula:

$$\% EE = 100 \times \frac{( with Triton value ) - ( without Triton value )}{( with Triton value )}$$

### Results and Conclusion

The particle size of final mRNA RBT26-LNP product was 127 nm with polydispersity index (PDI) of 0.11. The EE % was 94% and LNPs were stable after one freeze/thaw cycle. Test items (LNP-formulated RBT26 mRNA) were provided in vials of 0.04 mg/mL, 0.1 mg/mL and 0.3 mg/mL in dilution buffer, to be used without further dilutions.

Thus, the mRNA RBT26 vaccine candidate formulated in LNPs was successfully obtained.

### Example 14

In this Example, the production of the RBT26 protein used as comparative control in the present disclosure is described. The production of the RBT26 protein was as described in WO201757969, wherein the RBT26.1 (SEQ ID NO:1), RBT26.2 (SEQ ID NO:2) and RBT26.3 (SEQ ID NO:3) are referred as UL11, UL16 and UL21, respectively.

Briefly, RBT26.1, RBT26.2 and RBT26.3 in the form of a His-tagged trimer was expressed in Hi-5 insect cells and released from cell pellets after proper lysis. The trimer was subsequently purified using IMAC and a 0-500 mM imidazole buffer (Cat no J62593.AK, ThermoFisher, Switzerland) system (50 mM Hepes (Cat no 15630056, ThermoFisher, Switzerland), 500 mM NaCl (Cat no S1679, Sigma-Aldrich, Switzerland), pH 7.0, 1 mM TCEP (Cat no 75259, Sigma-Aldrich, Switzerland), 10% glycerol (Cat no G5516, Sigma-Aldrich, Switzerland)). Impurities were washed out by applying 25 mM imidazole to the column. The trimer was then eluted with 250 mM imidazole, followed by dialysis in Hepes buffer without imidazole (50 mM Hepes, 150 mM NaCl, pH 7.0, 0.5 mM TCEP, 10% glycerol).

Different fractions were collected during purification and were analyzed using SDS-PAGE gel electrophoretic analysis. FIG. 7 (b) shows an example of an SDS-PAGE gel electrophoretic analysis is shown. Lane 1: MW marker (Cat no 161-0363, BioRad, Switzerland), following lanes different fractions collected from purification. Lanes 13 and 14 are positive controls from two independent batches. FIG. 7 (c) shows an example of the Western Blot performed using a polyclonal anti-RBT26 serum from previous animal studies to detect protein UL11, UL16 and UL21 (also referred to herein as protein RBT26.1, RBT26.2 and RBT26.3, respectively). The first lane is the MW marker (Cat no 161-0385, BioRad, Switzerland) and the following lanes show purified protein at two different concentrations.

The protein RBT26 vaccine candidate composition was prepared as follows: For the low dose, (10) μg (8 μL) of Test item was further diluted in 142 μL PBS to a concentration of 67 μg/mL on the day of immunisation to a final volume of 150 μL. Then, 100 μL of 4 mg/mL CpG 1826 (cat. no. vac-1826-1, InvivoGen, France) was added to the solution. The capped bottle of Alhydrogel® adjuvant 2% (cat no. vac-alu-250, InvivoGen, France) was shook well before 250 μL Alhydrogel® adjuvant 2% was added to the antigen solution. The vaccine was mixed well by pipetting up and down for at least 5 minutes to allow Alhydrogel adjuvant 2% to effectively adsorb to the adjuvant.

For the high dose group, one hundred (100) μg (80 μL) of test item was further diluted in 70 μL PBS to a concentration of 670 μg/mL on the day of immunisation to a final volume of 150 μL. Then, 100 μL 4 mg/mL CpG 1826 will be added to the solution. The capped bottle of Alhydrogel® adjuvant 2% was shook well before 250 μL Alhydrogel® adjuvant 2% was added to the antigen solution. The vaccine was mixed well by pipetting up and down for at least 5 minutes to allow Alhydrogel adjuvant 2% to effectively adsorb to the adjuvant.

### Example 15

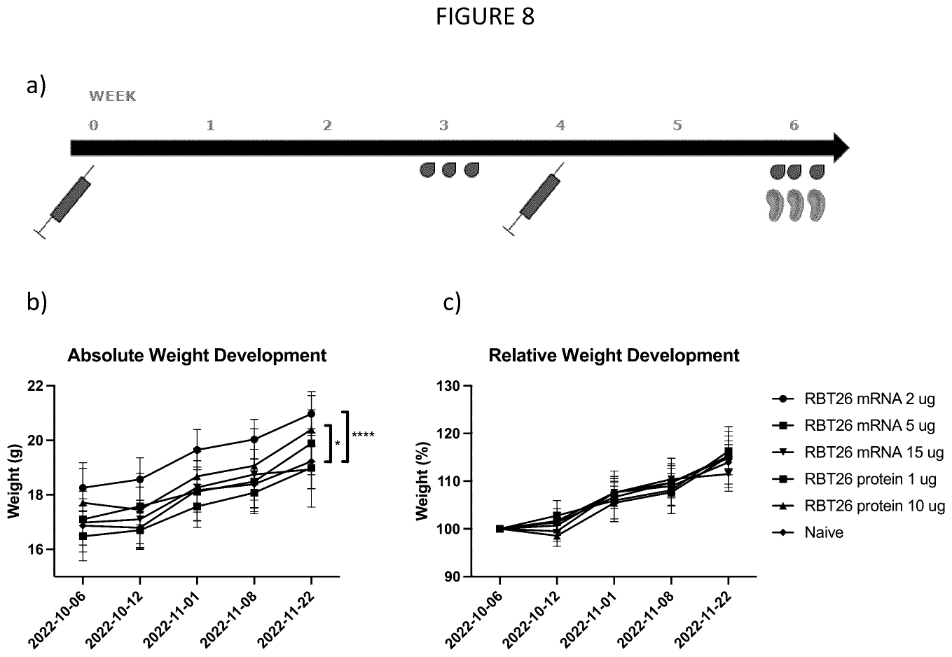

In this Example, an overview of the animal study is provided (see FIG. 8a).

Material and Method and Results

45 female BALB/cAnNCrl mice were obtained from Charles River, Germany and were randomized into six groups of eight or five animals per group to receive test item according to the Table below. Animals were acclimatized to the new environment before the initiation of the study for at least 5 days. The animals were subjected to an intramuscular (i.m.) injection of vaccine candidate, with adjuvant (as shown in Table 4a and 4b below).

TABLE 4a
Summary of information about animals used in study.

| | |
|---|---|
| Species, strain | Mouse, BALB/cAnNCrl (H2^{d}), Charles River Germany |
| Gender | Female. |
| Number | 45 |
| Source | Charles River, Germany |
| Justification of | Mice commonly used in vaccine studies |
| species | |
| Age | 6-7 weeks at arrival |
| Identification | The animals were identified by ID numbers (ID no. 001-045). The |
| | animals were marked cage-wise with ear markings. |
| Acclimatisation | Minimum 5 days prior to the commencement of the experiment. |

TABLE 4b
Summary of information about study groups.

| Group | Antigen | Adjuvant | Dose (ug) | Immunizations | n |
|---|---|---|---|---|---|
| 1 | mRBT 26 LNP | — | 2/total mRNA | 2 | 8 |
| 2 | mRBT 26 LNP | — | 5/total mRNA | 2 | 8 |
| 3 | mRBT 26 LNP | — | 15/total mRNA | 2 | 8 |
| 4 | pRBT 26 | CpG 1826 40 ug + | 1 protein | 2 | 8 |
| | | Alhydrogel 1% | | | |
| 5 | pRBT 26 | CpG 1826 40 ug + | 10 protein | 2 | 8 |
| | | Alhydrogel 1% | | | |
| 6 | Mock | — | — | 2 | 5 |

Vaccine administration was performed on Days 0 and 28 as outlined in Table 5 and shown in FIG. 8a. Animals were injected in the gastrocnemius muscle using an 29G insulin syringe (BD micro fine+insulin syringes 0.3 ml, cat. no. 324826, VWR).

TABLE 5
Summary of doses administered to animals in each study group.
CpG 1826 vaccigrade (cat. no. vac-1826-1, InvivoGen, France)
and alum (Alhydrogel adjuvant 2%, cat no. vac-alu-250,
InvivoGen, France) were used in groups 4 and 5.

| Schedule | Days 0 and 28 |
|---|---|
| Dose/ | Group 1: 40 μg/ml mRNA antigen |
| Concentration | Group 2: 100 μg/ml mRNA antigen |
| | Group 3: 300 μg/ml mRNA antigen |
| | Group 4: 20 μg/ml protein antigen + |
| | 0.8 mg/ml CpG + 1% Alum |
| | Group 5: 200 μg/ml protein antigen + |
| | 0.8 mg/ml CpG + 1% Alum |
| | Group 6: Naïve |
| Volume | 50 μl |

Mice were weighed, and the health status was noted prior to each administration and at termination. As shown in FIG. 8a, blood for the preparation of serum, was collected at day 21 and at termination. Animals were euthanized on day 42, whereupon terminal blood samples were collected for the preparation of serum, and spleens were excised. Thereafter animals were disposed of.

The blood collection sites were Vena saphena or retro-orbital plexus (at termination). Prior to collection from the orbital plexus, the animals were anaesthetized with isoflurane (Cat no 170579, Apoteket, Sweden). A volume of 0.1 ml of blood was collected at day 21 or as much as possible at day 42.

Collected samples were placed in BD SST Microtainer tubes (cat. no. 211-2166; VWR, Sweden) and kept at room temperature for at least 30 minutes. The samples were centrifuged at RT at 2000×g for 10 minutes. Serum was transferred to storage vials and frozen at −20° C. until analysis. At termination, spleens were dissected and placed in PBS (PBS, cat. No. 14190250, ThermoFisher, Sweden) and processed for analysis.

### Example 16

In this Example, the effect of vaccination with the mRNA vaccine candidate RBT26 according to the present disclosure or the comparative protein RBT26 vaccine on health status and body weight is compared. Different doses of the vaccine compositions are investigated and compared to naïve controls.

Material and Method and Results

Individual animal body weights were assessed prior to each administration and at termination. Statistical analyses were performed on actual and relative body weights (relative to the body weight recorded at arrival). The animals were randomised in such a way that, at arrival, two groups had slightly, but significantly higher absolute body weights than the untreated control group and kept the higher body weights throughout the experiment (FIG. 8b, repeated measures ANOVA). However, when relative bodyweights were analysed, where the initial body weight at the start of the in-life phase at the animal facility was 100%, no statistical differences in body weight development could be detected (FIG. 8c). No signs of decreased health status were reported during the in-life phase of the study, which was corroborated by the body weight development. The mice gained weight as expected during the study, and no significant deviations indicating a decreased health status could be detected.

### Conclusion

Thus, it was concluded that the administration of RBT26 mRNA vaccine and RBT26 protein vaccine had no negative effect of the health status of the animals and thus was well tolerated. Importantly, all doses of the RBT26 mRNA vaccine were equally well tolerated.

### Example 17

In this Example, the antibody responses after immunization with mRNA RBT26 vaccine and protein RBP26 vaccine were investigated using mice as a model system.

Material and Method

Groups of mice were immunized with 2, 5 or 15 mg of RBT26 mRNA formulated in LNPs or with 1 or 10 mg of RBT26 protein together with CpG 1826 (cat. no. vac-1826-1, InvivoGen, France) and alum (Alhydrogel adjuvant 2%, cat no. vac-alu-250, InvivoGen, France) as described above and serum IgG responses after the prime and boost immunizations were analyzed. Total antigen-specific IgG titers were determined in serum samples collected three weeks post the priming immunization and two weeks post boost by ELISA.

ELISA was performed as follows: ELISA plates (Nunc Polysorp, cat. no. 475094 ThermoFisher, Sweden) were coated with RBT26 protein antigen diluted in PBS (cat no. 14190250, ThermoFisher, Sweden), 100 μl/well. Plates were covered and stored overnight (o.n.) at 4° C., washed three times with PBS-Tween (0.05%) (Tween20: cat no P1379, Sigma-Aldrich, Sweden) and blocked with 5% milk (cat no. 70166-500 G, Sigma-Aldrich, Sweden) overnight (o.n.) at 4° C. or 2 hours at 37° C. All sera were diluted 1/100 in 2.5% milk in PBS-Tween as above. 100 μl 2.5% milk in PBS-Tween was added to all wells, except for the first well in the dilution series. 150 μl of 1/100 serum dilution was added to the first well followed by a serial dilution by transferring 50 μl to the following wells, resulting in an end volume for each well of 100 μl. Plates were incubated o.n. at 4° C. or 2 hours at 37° C. and washed 3 time with PBS-Tween (0.05%). 100 μl of HRP-conjugated anti mouse IgG antibody (Goat Anti-Rat IgG-HRP, cat. no. 3030-05 Southern Biotech, AH Diagnostics, Sweden) in 1.25% milk in PBS-Tween was added; incubated for 1.5 hours at 37° C. and subsequently washed five times with PBS-Tween. Substrate was added (OPD, cat. no. P9187-50SET, Sigma-Aldrich, Sweden) at 100 μl per well. The color development was topped with 50 μl of 1M HCL after 15 min at room temperature (RT). The absorbance at 490 nm was read immediately on a plate reader (SpectraMax plus, Molecular Devices). OD values were collected. Statistical analysis was performed using the non-parametric Kruskal-Wallis followed by Dunn's test for multiple comparisons. Statistical analyses were performed using the GraphPad Prism 9 software.

### Results and Conclusion

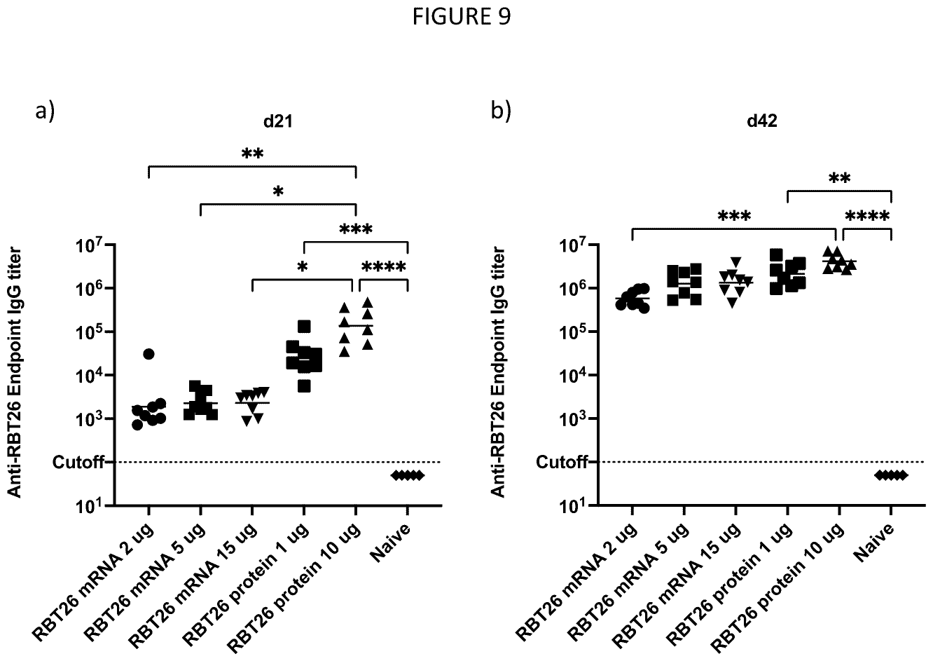

Anti-RBT26 IgG were elicited in all animals in all vaccinated groups after one single immunization, with the highest titres observed in serum obtained from animals treated with 10 μg protein antigen, which had significantly higher titres than all mRNA vaccinated groups (FIG. 9 a) after the priming immunization. After the second immunization, titers were increased in all animals approaching or exceeding end-point titers of 10⁶ in all groups. Again, the highest titers were elicited in the animals treated with 10 μg protein antigen formulated in alhydrogel and CpG1826, although the differences were small and only significant compared to the naïve control group and the lowest mRNA dose used (FIG. 9 b).

It was concluded that both mRNA RBT26 vaccine composition and protein RBT26 vaccine composition elicited anti-RBT26 antibodies at all doses tested after a single immunization. After the second immunization, animals administered the mRNA RBT26 vaccine composition exhibited end-point titers approaching or exceeding 10⁶, in line with the animals administered the protein RBT26 vaccine composition. Thus, the RBT26 mRNA vaccine and the RBT26 protein vaccines are able to elicit antibody responses to the same extent.

### Example 18

In this Example, the ability of the mRNA RBT26 vaccine and protein RBP26 vaccine to induce antigen-specific T cells was investigated using mice as a model system. Mice were injected with the vaccine intramuscularly according to a predefined schedule according to Table 4b and 5. Analysis of T cell responses was performed on lymphocytes isolated from spleens (referred to herein as splenocytes) from immunized mice. Cells were prepared and cultured with and without stimulants (a pool of predicted MHC class I and II binding peptides derived from RBT26 as defined in SEQ ID NO:35-51). SEQ ID NO:35-51 show the amino acid sequences of the predicted RBT26.1 derived MHC class I and II-binding peptides (SEQ ID NO:35), RBT26.2 derived MHC class I and II-binding peptides (SEQ ID NO:36-40) and RBT26.3 derived MHC class I and 11-binding peptides (SEQ ID NO:41-51). Detection of cytokines IFN-γ, IL-2, and TNF-α producing lymphocytes from the spleen (splenocytes) was performed using Flourospot analysis.

Material and Method

Preparation of splenocytes: Animals were euthanized and spleens were dissected and placed individually in PBS on ice. Each spleen was put in a 70 μm Falcon cell strainer (cat no. 352350/734-0003, VWR, Sweden) in a Petri dish, mashed, 5 ml complete RPMI-1640 (cat no. 21875-034, ThermoFisher, Sweden) was added and the cells were washed through the strainer two times. Cell suspension was collected, centrifuged at 350×g for 5 minutes and subsequently, the supernatant was discarded and the cells were resuspended. The cells were lysed in 1 ml red blood cell lysis buffer (cat no. R7757-100 ml, Sigma-Aldrich, Sweden) for 2-3 minutes and 9 ml complete RPMI-1640 was added, followed by and centrifugation at 350×g for 5 minutes. The supernatant was discarded and the cells were resuspended and 2.5 ml of complete RPMI was added. Cells were diluted 1:10 in a 96 well plate (cat no. 83.3924.300, Sarstedt, Sweden) in complete RPMI-1640 and counted on a Guava EasyCyte cell counter (Merck-Millipore) according to standard procedures. Cells were diluted cells to a final concentration of 2×10⁶ cells per ml in complete RPMI-1640 and a total volume of 3 ml was place into individual tubes.

Stimulation of splenocytes: 96-well plates (supplied in Fluorospot kit Mouse FluoroSpot Plus Mouse IFN-γ/IL-2/TNF-α; Cat no. FSP-414245-10, Mabtech, Sweden) were washed 3 times with sterile PBS 200 μl/well and PBS was removed; blocking was performed with 200 al complete RPMI-1640/well for >30 min at RT. Antigens (peptides (SEQ ID NO:35-51) and positive control (Concanavalin A (ConA), cat no C0412, Sigma-Aldrich, Sweden) were diluted in complete RPMI-1640 to a working concentration of 4 ag/ml and 100 μl of peptide antigen or ConA was added per well. The peptide antigens were prepared as follows:
4 mg of lyophilized antigen peptide (SEQ ID NO:35-51, obtained from GenScript, the Netherlands) was dissolved in 200 μl DMSO. When the peptides were completely dissolved, 800 μl of PBS was added. After thorough mixing, aliquots of 50 μl were made and stored at −20° C. until use. At use, peptides were diluted from stock in complete RPMI-1640 to a working concentration of 4 μg/ml.

Next, 100 μl of cell suspension was added to each well in triplicate for each stimulation antigen. As controls 100 μl complete RPMI-1640/well was used instead the cell suspension. Plates were incubated at 37° C., 5% CO₂, for 44±2 hours.

Detection antibodies (as specified below in Table 7) were diluted in PBS containing 0.1% BSA.

TABLE 7
The antibodies were diluted according to the table in PBS + 0.1% BSA.
Mouse FluoroSpot Plus: Mouse IFN-γ/IL-2/TNF-α
kit from Mabtech (cat no. FSP-414245-10) was used.

| | Detection | | |
|---|---|---|---|
| Kit | antibodies | Concentration | Dilution |
| IFN-γ/IL-2/TNF-α | anti-IFN-γ mAb R4- | 1 mg/ml | 1:200 |
| | 6A2-BAM | | |
| IFN-γ/IL-2/TNF-α | anti-IL-2 mAb 5H4, | 1 mg/ml | 1:500 |
| | biotinylated | | |
| IFN-γ/IL-2/TNF-α | anti-TNF-α mAb | 1 mg/ml | 1:200 |
| | MT11B10-WASP | | |

Cells were flicked out and wells were washed 5 times with 200 μl PBS/well. 100 μL per well of the detection antibody mix was added and incubated for 2 hours±5 minutes at RT. Solution containing antibodies were removed and wells were washed 5 times with 200 PBS/well. 100 μL per well of the diluted fluorophore conjugates (see Table 8) (1:200 in PBS containing 0.1% BSA) added and incubated at RT for 1 hour±5 minutes at RT, followed by removal of fluorophore conjugate solution subsequent washing 5 times with 200 PBS/well.

TABLE 8
The fluorophore conjugates were
diluted 1/200 in PBS + 0.1% BSA.

| Kit | Flourophore conjugate | Dilution |
|---|---|---|
| IFN-γ/IL-2/TNF-α | anti-BAM-490 | 1:200 |
| IFN-γ/IL-2/TNF-α | SA550 | 1:200 |
| IFN-γ/IL-2/TNF-α | anti-WASP-640 | 1:200 |

Into each well, 50 μL Fluorescence enhancer provided in kit (cat no. FSP-414245-10, Mabtech, Sweden) was added and left for 10±5 minutes at RT. Fluorescence enhancer as removed and the bottom of the plate was removed to allow the plate to dry completely and was protected from light to avoid fluorophore fading. Plates were stored in the dark and subsequently read in the IRIS plug and play automated Fluorospot reader (FluoroSpot reader (IRIS), Mabtech Plate evaluation Service) using the RAWspot technology analysis software package (Mabtech, Sweden).

The average of triplicates was calculated for each individual mouse and stimulation antigen. The average was multiplied by 5 and results were reported as spot forming cells (SFC) per million splenocytes for each peptide and medium control.

### Results and Conclusion

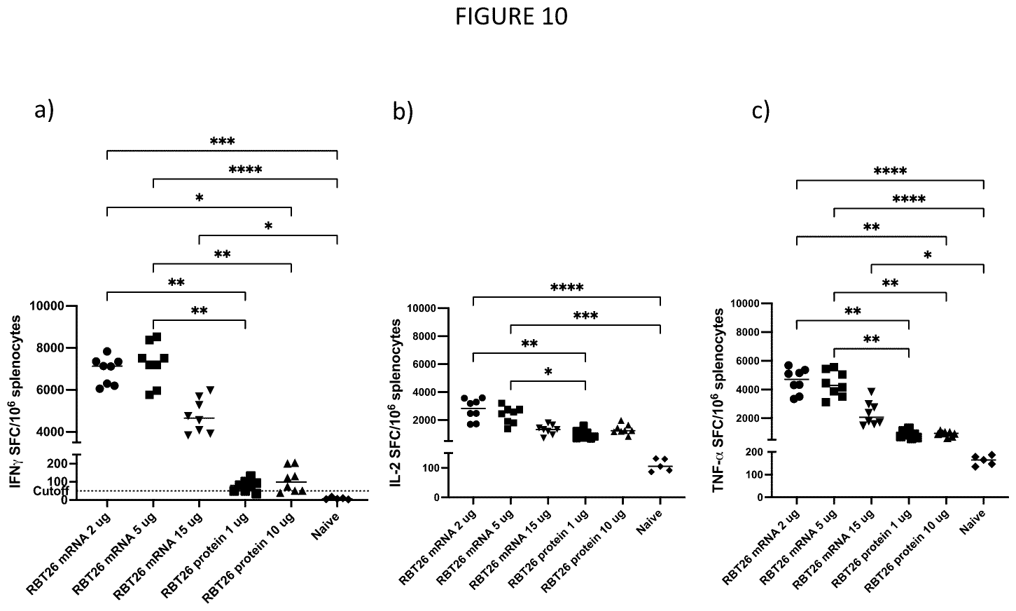

Secretion of IFN-γ, IL-2, and TNF-α was determined by Fluorospot analysis after restimulation of splenocytes with a pool of 17 predicted RBT26-derived MHC class I and II-binding peptides (SEQ ID NO:35-51) (GenScript, the Netherlands). These peptides bind efficiently to MHC class I and MHC class II without need for processing and they do not contain potentially immune activating impurities of recombinant proteins, and were not used for the immunization. Accordingly, the peptide pool containing predicted BALB/c MHC class I and II epitopes was very efficient at restimulating secretion of cytokines, especially from the mRNA vaccinated groups. The number of IFN-γ spot forming cells was the highest in T cells isolated from animals administered the 2 and 5 μg doses of the mRNA RBT26, which animals showed significantly higher numbers of IFN-γ, IL-2 and TNF-α spot forming cells compared to T cells obtained from animals adminstered with 1 μg of protein RBT26 vaccine plus adjuvant, and significantly more IFN-γ and TNF-α compared to the administered 10 μg of protein RBT26 vaccine plus adjuvant (FIG. 10). The 15 μg dose of RBT26 mRNA induced more IFN-γ and TNF-α SFC compared the animals adminstered the protein RBT26 vaccine.

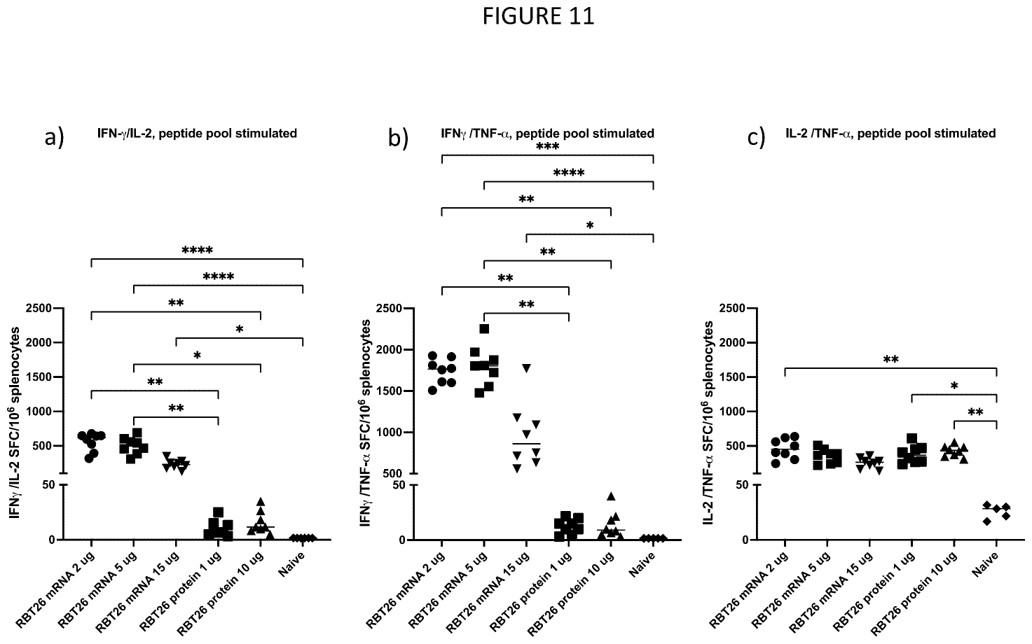

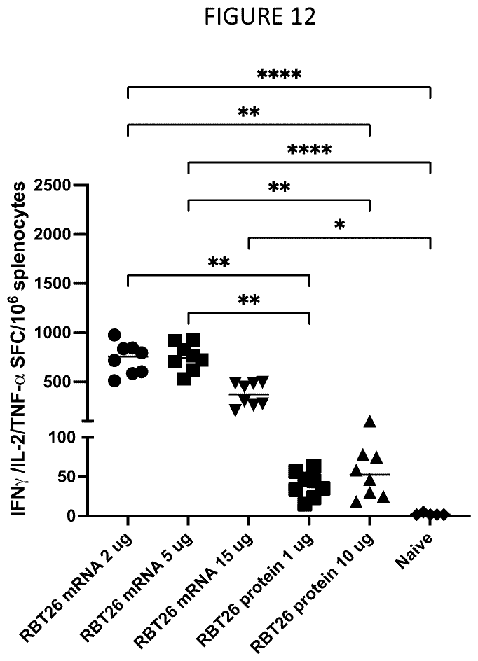

Surprisingly, the present inventors found that immunization with mRNA RBT26 vaccine candidate elicited significantly more multifunctional T cells that produced more than one cytokine: IFN-γ/IL-2; IFN-γ/TNF-α (FIG. 11) and IFN-γ/IL-2/TNF-α (FIG. 12) in contrast to immunization with the protein RBT26 vaccine candidate. Importantly, polyfunctional HSV 1 or 2-specific T cells have been implicated as an important factor for immunologic control of herpes virus infections (review in Troung et al, 2019; Egan et al., 2013, Khan et al., 2017 and Kuo et al., 2014).

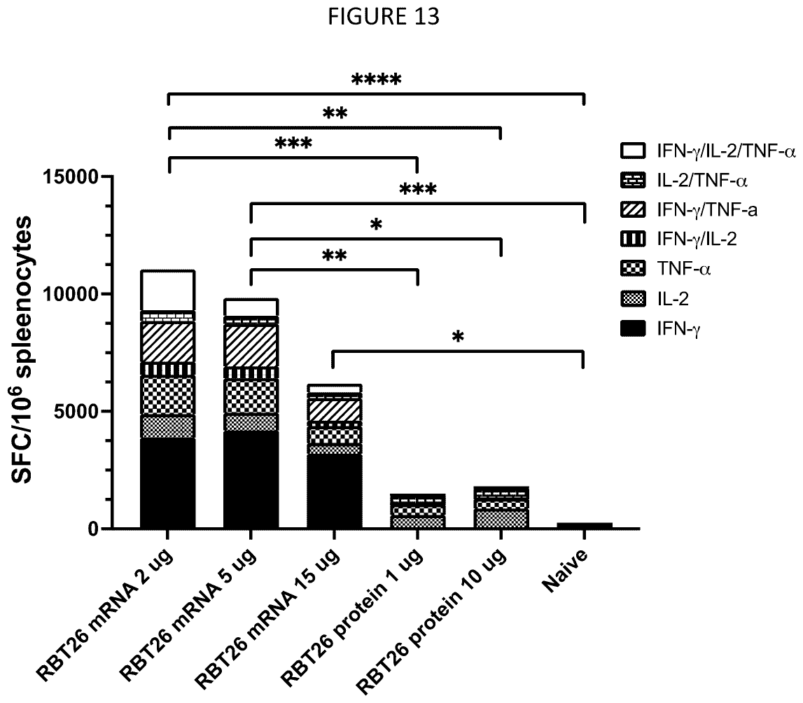

The combined T cell responses (FIG. 13) after peptide restimulation indicate that protein vaccination elicited significantly fewer vaccine induced T cells.

The data shows that administration of the mRNA RBT26 vaccine candidate drove very strong induction of antigen-specific T cells, including secretion of IFN-γ and large numbers of polyfunctional T cells that secreted two or three of the cytokines measured. Conversely, administration of the protein RBT26 vaccine candidate plus adjuvant elicited very few IFN-γ spot forming cells and most T cells produced only single cytokines or a combination of IL-2 and TNF-α. The two vaccine types elicited IL-2 and TNF-α producing T cells in comparable numbers. Interestingly, polyfunctional T cells secreting all three cytokines were absent in mice adminstered protein RBT26 vaccine candidate.

TABLE 9
Summary of results from Fluorospot analysis. Number of SPC (spot
forming cells) per million splenocytes is reported with standard
error of the mean (SEM) for each vaccinated group.

| | RBT26 mRNA | | RBT26 mRNA | | RBT26 mRNA | |
|---|---|---|---|---|---|---|
| | 2 μg | | 5 μg | | 15 μg | |
| | SFC/million | SEM | SFC/million | SEM | SFC/million | SEM |
| Cytokine(s) | splenocytes | (+/−) | splenocytes | (+/−) | splenocytes | (+/−) |
| IFNγ | 3888 | 156 | 4190 | 187 | 3201 | 259 |
| IL-2 | 1001 | 133 | 746 | 96 | 450 | 42 |
| TNF-α | 1670 | 194 | 1483 | 169 | 728 | 106 |
| IFNγ/IL-2 | 556 | 47 | 502 | 44 | 229 | 24 |
| IFNγ/TNF-α | 1738 | 54 | 1808 | 86 | 958 | 140 |
| IL-2/TNF-α | 457 | 51 | 352 | 37 | 250 | 27 |
| IFNγ/IL-2/TNF-α | 1738 | 54 | 752 | 110 | 370 | 42 |
| Total cytokine | 11048 | 481 | 9832 | 623 | 6186 | 381 |
| producing cells | | | | | | |
| | RBT26 protein | | RBT26 protein | | | |
| | 1 ug | | 10 ug | | Naive | |
| | SFC/million | SEM | SFC/million | SEM | SFC/million | SEM |
| Cytokine(s) | splenocytes | (+/−) | splenocytes | (+/−) | splenocytes | (+/−) |
| IFNγ | 15 | 3 | 26 | 8 | 7 | 3 |
| IL-2 | 573 | 65 | 844 | 95 | 81 | 7 |
| TNF-α | 460 | 40 | 425 | 25 | 135 | 10 |
| IFNγ/IL-2 | 10 | 3 | 16 | 4 | 1 | 0 |
| IFNγ/TNF-α | 13 | 2 | 14 | 4 | 0 | 0 |
| IL-2/TNF-α | 379 | 45 | 428 | 28 | 26 | 3 |
| IFNγ/IL-2/TNF-α | 40 | 6 | 54 | 10 | 1 | 1 |
| Total cytokine | 1491 | 149 | 1807 | 146 | 252 | 9 |
| producing cells | | | | | | |

In conclusion, it was surprisingly found that the mRNA RBT26 vaccine candidate can elicit very strong induction of antigen-specific T cells, including secretion of IFN-γ and large numbers of polyfunctional T cells, in contrast to the protein RBT26 vaccine candidate plus adjuvant. It was concluded that a mRNA RBT26 vaccine is expected to provide superior protection against disease/initial infection, as well as provide improved immunological control of HSV-2 infection in already infected patients.

### Example 19

In this Example intracellular cytokine staining (ICS) for IFN-γ of peptide restimulated CD4+ and CD8+ T cells was performed.

Material and Method

Splenocytes were prepared as described in Example 8. Obtained splenoctes were resuspended in cell culture media (supplier) at 2.5×10⁶ cells/ml. The splenocytes were seeded 1 ml per well into wells of a 24 well plate (Cat no. 83.3922.300, Sarstedt, Sweden) and either left unstimulated or stimulated with peptides (SEQ ID NO:35-51) as described above.

After stimulation for 40 h, protein transport inhibitor (cocktail of Monensin and Brefeldin A, cat. no. 00-4980-03, eBioscience, ThermoFisher, Sweden) was added and the cells were incubated an additional 6 hours. Cells were harvested and stained with anti-CD8a-PerCP-Cy5.5 (cat. no. 45-0081-82, ThermoFisher, Sweden) and anti-CD4-APC (cat no. 51-0042-82, ThermoFisher, Sweden) for 30 minutes at 4° C., then were washed after which they were fixed and permeabilized with Intracellular Fixation & Permeabilization buffer (cat. no. 88-8824-00, eBioscience, ThermoFisher, Sweden) for 30 minutes. Cells were washed with permeabilization buffer and then stained with anti-IFN-γ-PE (cat. no. 12-7311-82, ThermoFisher, Sweden) diluted in permeabilization buffer for 30 minutes at RT while protected from light. Finally, cells were washed with permeabilization buffer and then resuspended in PBS and analyzed on a Guava EasyCyte HT8 flow cytometer (Merck-Millipore).

### Results and Conclusion

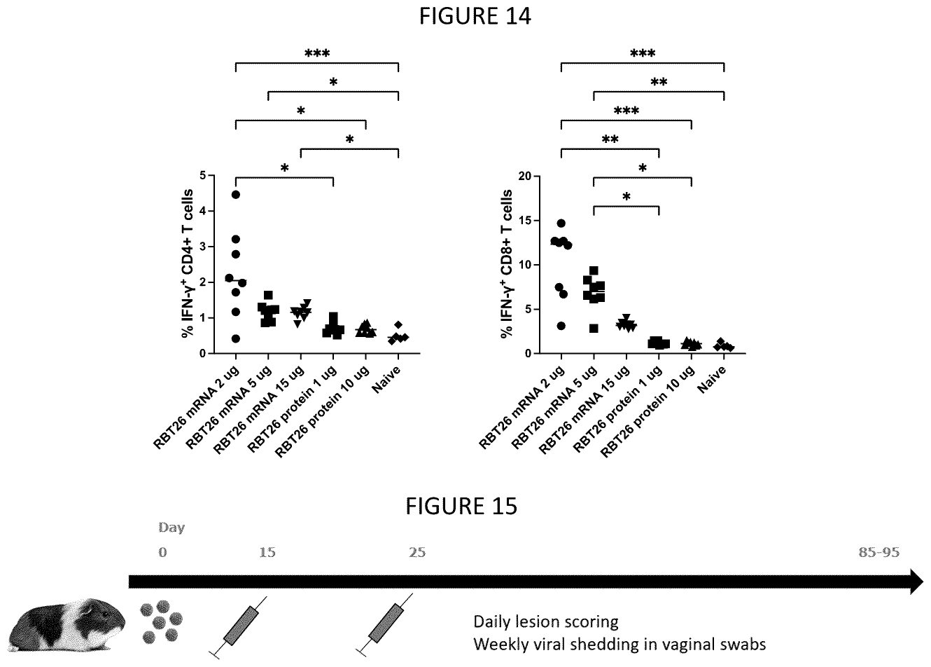

Intracellular cytokine staining (ICS) for IFN-γ of peptide restimulated T cells obtained from all animals showed a strong induction of IFN-γ in T cells from animals administered 2 and 5 μg RBT26 mRNA vaccine candidate (FIG. 14). IFN-γ production was only detected in T cells from animals administered RBT26 mRNA vaccine candidate, thus confirming the findings in the Fluorospot analysis. Further analysis revealed that both CD4+ and CD8+ T cells were responsible for the IFN-γ secretion. The mice immunized with the lowest mRNA RBT26 dose (2 μg) had significantly higher frequencies of IFN-γ producing CD4+ T cells as compared to T cells from animals immunized with the protein RBT26 vaccine candidate. The frequencies of vaccine-specific CD8+ T cells in the animals administered 2 and 5 μg RBT26 mRNA vaccine candidate were very high and significantly higher as compared to the protein vaccinated animals.

In conclusion, administration of the mRNA RBT26 vaccine candidate elicited antigen-specific CD4+ and CD8+ T cells, as determined by intracellular cytokine staining for IFN-γ production. The CD8+ T cell responses were very strong consisting of up to 14% of the total pool of CD8+ T cells in the spleen. In particular, low and intermediate mRNA doses were more efficient at eliciting T cell responses compared to the highest dose tested.

### Concluding Remarks for Examples 12-19

The present data shows that immunization with mRNA RBT26 vaccine candidate according to the present invention and with the comparative protein RBT26 vaccine candidate are well tolerated by the treated animals, as indicated by growth curves and visual inspections during the in-life phase of the study. This conclusion was further supported by the relative body weight curves, indicating highly similar body weight development between groups as defined in Table 4b expressed as a percentage relative to the original weight of each animal. Thus, the immunization with the mRNA RBT26 vaccine candidate according to the present invention and protein RBT26 vaccine candidate did not have any negative impact on the health status or body weight of the animals.

Both immunization with mRNA RBT26 vaccine candidate and protein RBT26 vaccine candidate elicited anti-RBT26 antibodies at all doses tested after a single immunization. All vaccinated mice had detectable antibodies in serum already after the first immunization, although the high dose of the protein vaccine (10 mg) induced significantly higher titers after the priming immunization. After the booster immunization, serum anti-RBT26 IgG responses were increased in all animals immunized with mRNA RBT26 vaccine candidate or protein RBT26 vaccine candidate and endpoint titers reached and in some animals exceeded 106.

Thus, the immunization with mRNA RBT26 vaccine candidate according to the present invention was well tolerated and induced significant IgG responses after prime and boost vaccination. Importantly, in constrast to protein RBT26 vaccine candidate, mRNA RBT26 vaccine candidate surprisingly showed beneficial effects in terms of T cell response after immunization.

It is known that T cells are crucial for the immunologic control of HSV reactivation, and play several key roles in the immune response. Thus, the provision of a vaccine against HSV-2 which elicits strong T cell response is particularly desirable. CD4+ T cells are critical for the activation of B cells and antibody class-switching, as well as for “licensing” DCs to activate CD8+ T cells. They also secrete IFN-γ which performs a number of antiviral roles including: (i) limiting HSV viral replication by the induction of antiviral genes such as PKR, which inhibits translation within infected cells; (ii) restores HSV-induced MHC class I downregulation; and (iii) stimulates epithelial cell CXCL9 and CXCL10 production, which recruits CD8+ T cells to the site of infection. CD8+ T cells have the important role of killing virally infected cells via their cytotoxic components (e.g. perforin and granzyme), mediated through the engagement of MHC class I molecules presenting viral peptides on target cells. It has been shown that in HSV infection, CD4+ and CD8+ T cells surround the neurons and adherent satellite cells of trigeminal ganglia and can control latency and reactivation. Activated (CD69+) HSV-specific effector memory CD4+ and CD8+ T cells expressing IFN-γ, TNF and CCL5 are found in HSV infected ganglia and around neurons, and that CD8 T cells initially clear active lesions, then become T_{RM} cells, that ensure reactivation is a rare occurrence through high killing efficiency and IFN-γ production (reviewed Troung et al, supra). The present inventors have found that the mRNA RBT26 vaccine candidate according to the present invention as well as the protein RBT26 vaccine candidate elicited antigen-specific T cells, however surprisingly the administration of the mRNA RBT26 vaccine candidate induced very potent induction of IFN-γ secretion, which was significantly higher than responses elicited by protein vaccination. In addition, administration of the mRNA RBT26 vaccine candidate stimulated more IL-2 and TNF-α secretion than the protein RBT26 vaccine candidate. Importantly, administration of the mRNA RBT26 vaccine candidate elicited significantly more multifunctional T cells that produced more than one cytokine: IFN-γ/IL-2; IFN-γ/TNF-α and IFN-γ/IL-2/TNF-α. Notably, administration of the mRNA RBT26 vaccine candidate thus elicited several types of multifunctional T cells, including bifunctional and trifunctional T cells. Multifunctional HSV-specific T cells have been implicated as an important factor for immunologic control of herpes virus infections (Srivastava et al., 2018). Thus, the ability of a vaccine candidate to elicit multifunctional T cell response is particularly beneficial. Furthermore, administration of the mRNA RBT26 vaccine candidate elicited antigen-specific CD4+ and CD8+ T cells, as determined by intracellular cytokine staining for IFN-γ production, and CD4 and CD8 surface expression analyzed by flow cytometry. The CD8+ T cell responses were very strong consisting of up to 14% of the total pool of CD8+ T cells in the spleen. It is further surprising that low and intermediate mRNA doses were more efficient at eliciting T cell responses than the highest dose tested in the present study.

The present inventors surprisingly found that the mRNA RBT26 vaccine candidate according to the present disclosure is superior to its protein counterpart as evidenced by the data presented herein. Moreover, it is expected that the mRNA RBT vaccine will efficiently improve immunologic control of herpes virus infections and thus improve control of reoccurring reactivation of the latent virus.

### Example 20

This example describes an in vivo study in guinea pigs comprising an HSV-2 challenge followed by therapeutic vaccination with mRNA RBT26 vaccine candidate as described herein and with the comparative protein RBT26 vaccine.

Material and Method

40 female 5-6 weeks of age Hartley strain guinea pigs from a commercial breeder are randomized and divided into four groups of ten individuals. After one week of acclimatization, animals are infected by intravaginal inoculation with 200 mL of a suspension containing 5×10⁵ PFU of HSV-2 strain MS (Cat no VR-540, ATCC, USA). Once acute infection is resolved, latently infected animals are vaccinated intramuscularly twice, in the right hind calf muscle on day 15 and on day 25 post-infection.

One group of guinea pigs is vaccinated with 5 μg RNA consisting of equimolar amounts of pseudouridine modified RBT26.1 (SEQ ID NO:26), RBT 26.2 (SEQ ID NO:27) and RBT26.3 (SEQ ID NO:28) mRNA formulated in LNPs essentially as described in Example 2 (Genvoy ILM, Precision Nanosystems, Canada) by intramuscular injection of 50 μL vaccine. A second group is vaccinated with 20 μg RNA consisting of equimolar amounts of pseudouridine modified RBT26.1 (SEQ ID NO: 26), RBT 26.2 (SEQ ID NO:27) and RBT26.3 (SEQ ID NO:28) mRNA formulated in LNPs essentially as described in Example 2 (Genvoy ILM, Precision Nanosystems, Canada) by intramuscular injection of 50 μL vaccine. The comparator group is immunized with 10 μg of RBT26 proteins mixed with 100 μg CpG and 150 μg alum (CpG oligonucleotide (5′-TCGTCGTTGTCGTTTTGTCGTT-3′ (SEQ ID NO:52)), cat no tlrl-2007, Invivogen, France; and Alhydrogel, cat no. vac-alu-250, InvivoGen, France) by intramuscular injection of 50 μL vaccine. The comparative protein RBT26 vaccine is prepared essentially as described in Example 4. Finally, a negative control group is vaccinated with mRNA/LNP diluent (Precision Nanosystems, Canada) by intramuscular injection of 50 μL.

Guinea pigs are examined for vaginal lesions and were recorded for each individual animal on a daily basis on a scale of 0 to 4, where 0 reflects no disease, 1 reflects redness, 2 reflects a single lesion, 3 reflects coalesced lesions, and 4 reflects ulcerated lesions. Observations are carried out starting right after second immunization and throughout the study until study end at day 90 post infection. Vaginal swabs are collected weekly using a Dacron swab (cat no. dacroswab type 1; Spectrum Laboratories, USA) starting from day 0, day 2, day 7 and thereafter on a weekly basis until the end of the study. Individual swabs are transferred to a 2 mL sterile cryogenic vial containing 1 ml culture medium and stored at −80° C. until use. The HSV-2 DNA copy numbers in the individual samples are then determined by quantitative PCR. DNA is isolated from 300 μl of guinea pig vaginal swab material using DNeasy blood and tissue kits (Cat no 69504, Qiagen, Germany). HSV-2 DNA copy number was determined using purified HSV-2 DNA (cat no 17-922-500, Advanced Biotechnologies, USA) and based on a standard curve that is generated with 50,000, 5,000, 500, 50, and 5 copies of DNA and run in triplicates. Each guinea pig sample is analyzed in duplicate. Samples with <150 copies/ml by 40 cycles or only positive in one of two wells are reported as negative. Primer and probe sequences for HSV-2 Us9 are: primer forward, 5′-GGCAGAAGCCTACTACTCGGAAA-3′ (SEQ ID NO:53), and reverse 5′-CCATGCGCACGAGGAAGT-3′(SEQ ID NO:54), and probe with reporter dye 5′-FAM-CGAGGCCGCCAAC-MGBNFQ-3′ (FAM, 6-carboxyfluorescein, custom made, ThermoFisher, Sweden) (wherein the reporter dye is defined by a DNA sequence region according to SEQ ID NO:55 and an amino acid sequence region according to SEQ ID NO:85). All reactions are performed using Applied Biosystems TaqMan gene expression master mix (Cat no 4369016, ThermoFisher, Sweden) and data is collected and analyzed on StepOnePlus real time PCR system (Cat no 4376600, ThermoFisher, Sweden).

### Results and Conclusion

It is expected that a latent infection is established in all of the mock vaccinated negative control animals, resulting in virus release into the vaginal lumen and that genital lesions occur repeatedly during the course of the experiment. Importantly, it is also expected that vaccination will reduce the number of viral recurrences as assessed by frequency and clinical score of the genital lesions, as well as viral secretion into the genital lumen. The present inventors expect that the RBT26 mRNA vaccine will be superior to the RBT26 protein vaccine given its surprising ability to elicit high magnitude of polyfunctional T cells in preclinical testing in mice.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention.

Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes. Other embodiments are within the following claims. In addition, where features or aspects of the invention are described in terms of Markush groups, those skilled in the art will recognize that the invention is also thereby described in terms of any individual member or subgroup of members of the Markush group.

### ITEMIZED LIST OF EMBODIMENTS

1. An immunogenic composition capable of eliciting a Herpes Simplex Virus 2 (HSV-2)-specific immune response in a subject when administered to said subject, wherein said immunogenic composition comprises at least one, such as at least two, such as all three nucleic acid(s) selected from the group consisting of:
   (i) a nucleic acid encoding a UL11 protein of HSV-2 or an immunogenic fragment thereof,
   (ii) a nucleic acid encoding a UL16 protein of HSV-2 or an immunogenic fragment thereof, and
   (iii) a nucleic acid encoding a UL21 protein of HSV-2 or an immunogenic fragment thereof, wherein said UL11 protein comprises an amino acid sequence having at least 80% identity to SEQ ID NO:1, said UL16 protein comprises an amino acid sequence having at least 80% identity to SEQ ID NO:2, and said UL21 protein comprises an amino acid sequence having at least 80% identity to SEQ ID NO:3.
2. The immunogenic composition according to item 1, wherein said UL11 protein of HSV-2 comprises or consists of an amino acid sequence selected from a group consisting of SEQ ID NO:1 and any amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, identity to SEQ ID NO:1.
3. The immunogenic composition according to item 1 or 2, wherein said UL11 protein of HSV-2 comprises or consists of an amino acid sequence according to SEQ ID NO:1.
4. The immunogenic composition according to any one of the preceding items, wherein said UL16 protein of HSV-2 comprises or consists of an amino acid sequence selected from a group consisting of SEQ ID NO:2 and any amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, identity to SEQ ID NO:2.
5. The immunogenic composition according to any one of the preceding items, wherein said UL16 protein of HSV-2 comprises or consists of an amino acid sequence according to SEQ ID NO:2.
6. The immunogenic composition according to any one of the preceding items, wherein said UL21 protein of HSV-2 comprises or consists of an amino acid sequence selected from a group consisting of SEQ ID NO:3 and any amino acid sequence having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, identity to SEQ ID NO:3.
7. The immunogenic composition according to any one of the preceding items, wherein said UL21 protein of HSV-2 comprises or consists of an amino acid sequence according to SEQ ID NO:3.
8. The immunogenic composition according to any one of the preceding items, wherein each nucleic acid as defined in i), ii) and/or iii) is present in an individual nucleic acid construct or wherein at least two of said nucleic acid as defined in i), ii) and/or iii) are present in the same nucleic acid construct, such as wherein all three of said nucleic acid as defined in i), ii) and/or iii) are present in the same nucleic acid construct.
9. The immunogenic composition according to item 7, wherein each nucleic acid as defined in i), ii) and/or iii) is present in an individual nucleic acid construct.
10. The immunogenic composition according to any one of the preceding items, wherein the nucleic acid as defined in i), ii) and/or iii) is selected from the group consisting of DNA and RNA.
11. The immunogenic composition according to item 10, wherein the nucleic acid as defined in i), ii) and/or iii) is DNA.
12. The immunogenic composition according to any one of the preceding items, wherein the nucleic acid as defined in i), ii) and/or iii) is inserted into a plasmid or a vector.
13. The immunogenic composition according to item 12, wherein the nucleic acid as defined in i), ii) and/or iii) is inserted into a vector, such as a viral vector.
14. The immunogenic composition according to item 13, wherein said viral vector is an adenoviral vector.
15. The immunogenic composition according to any one of items 1 to 10, wherein the nucleic acid as defined in i), ii) and/or iii) is RNA, such as mRNA.
16. The immunogenic composition according to item 10 or 15, wherein said RNA is obtained by an RNA manufacturing method, such as by in vitro transcription.
17. The immunogenic composition according to any one of items 1 to 10, 15 and 16, wherein the nucleic acid as defined in i) comprises a nucleic acid sequence selected from a group consisting of SEQ ID NO:16 and any codon-optimized and/or nucleoside modified version thereof, such as wherein the nucleic acid as defined in i) comprises a nucleic acid sequence selected from a group consisting of SEQ ID NO:16 and SEQ ID NO:32.
18. The immunogenic composition according to item 17, wherein the nucleic acid as defined in i) comprises a nucleic acid sequence selected from any codon-optimized or nucleoside modified version of SEQ ID NO:16, such as any codon-optimized and nucleoside modified version of SEQ ID NO:16, such as any nucleoside modified version of SEQ ID NO:32.
19. The immunogenic composition according to item 17 or 18, wherein the nucleic acid as defined in i) comprises a nucleic acid sequence according to SEQ ID NO:32 or SEQ ID NO:86.
20. The immunogenic composition according to any one of items 1 to 10 and 15 to 19, wherein the nucleic acid as defined in ii) comprises a nucleic acid sequence selected from a group consisting of SEQ ID NO:17 and any codon-optimized and/or nucleoside modified version thereof, such as wherein the nucleic acid as defined in ii) comprises a nucleic acid sequence selected from a group consisting of SEQ ID NO:17 and SEQ ID NO:33.
21. The immunogenic composition according to item 20, wherein the nucleic acid as defined in ii) comprises a nucleic acid sequence selected from any codon-optimized or nucleoside modified version of SEQ ID NO:17, such as any codon-optimized and nucleoside modified version of SEQ ID NO:17, such as nucleoside modified version of SEQ ID NO:33.
22. The immunogenic composition according to item 20 or 21, wherein the nucleic acid as defined in ii) comprises a nucleic acid sequence according to SEQ ID NO:33 or SEQ ID NO:87.
23. The immunogenic composition according to any one of items 1 to 10 and 15 to 22, wherein the nucleic acid as defined in iii) comprises a nucleic acid sequence selected from a group consisting of SEQ ID NO:18 and any codon-optimized and/or nucleoside modified version thereof, such as wherein the nucleic acid as defined in iii) comprises a nucleic acid sequence selected from a group consisting of SEQ ID NO:18 and SEQ ID NO:34.
24. The immunogenic composition according to item 21, wherein the nucleic acid as defined in iii) comprises a nucleic acid sequence selected from any codon-optimized or nucleoside modified version of SEQ ID NO:18, such as any codon-optimized and nucleoside modified version of SEQ ID NO:18, such as nucleoside modified version of SEQ ID NO:34.
25. The immunogenic composition according to item 23 or 24, wherein the nucleic acid as defined in iii) comprises a nucleic acid sequence according to SEQ ID NO:34 or SEQ ID NO.88.
26. The immunogenic composition according to any one of items 15 to 25, wherein the nucleic acid as defined in i), ii) and/or iii) is a nucleoside modified mRNA comprising one or more modified nucleosides, such as a nucleoside modified mRNA comprising one or more pseudouridine residues.
27. The immunogenic composition according to item 26, wherein said one or more modified nucleosides are selected from the group consisting of 5-methoxyuridine, 5-methylcytidine (m5C), N6-methyladenosine (m6A), 5-methyluridine (m5U), 2-thiouridine (s2U), 2′-0-methylguanosine (m2,2G), 2′-0-methylcytidine (m2,2C), 2′-0-methyluridine (m2,2U), 5-methoxycytidine (m5oC), 1-methylpseudouridine (m1ψ), 1-methyl-3-(3-amino-5-carboxypropyl)pseudouridine (m¹acp³ψ), 2′-0-methylpseudouridine (Wm), 5-methyldihydrouridine (m⁵D) and (3-methylpseudouridine (m³ψ).
28. The immunogenic composition according to any one of items 15 to 27, wherein the nucleic acid as defined in i), ii) and/or iii) comprises a poly-A tail.
29. The immunogenic composition according to item 28, wherein said poly-A tail comprises a nucleic acid sequence according to SEQ ID NO:58.
30. The immunogenic composition according to any one of items 15 to 29, wherein the nucleic acid as defined in i), ii) and/or iii) comprises a 5′ untranslated region, such as wherein said 5′ untranslated region enhances translation.
31. The immunogenic composition according to item 30, wherein said 5′ untranslated region comprises a nucleic acid sequence according to SEQ ID NO:59.
32. The immunogenic composition according to any one of items 15 to 31, wherein the nucleic acid as defined in i), ii) and/or iii) comprises a 3′ untranslated region, such as wherein said 3′ untranslated region enhances translation.
33. The immunogenic composition according to item 32, wherein said 3′ untranslated region comprises a nucleic acid sequence according to SEQ ID NO:60.
34. The immunogenic composition according to any one of items 15 to 33, wherein the nucleic acid as defined in i) comprises a nucleic acid sequence according to SEQ ID NO:61 or SEQ ID NO:26.
35. The immunogenic composition according to any one of items 15 to 34, wherein the nucleic acid as defined in ii) comprises a nucleic acid sequence according to SEQ ID NO:62 or SEQ ID NO:27.
36. The immunogenic composition according to any one of items 15 to 35, wherein the nucleic acid as defined in iii) comprises a nucleic acid sequence according to SEQ ID NO:63 or SEQ ID NO:28.
37. The immunogenic composition according to any one of items 15 to 36, wherein the nucleic acid as defined in i), ii) and/or iii) comprises an mRNA cap.
38. The immunogenic composition according to item 37, wherein said mRNA cap is an m7GpppG cap, a 3′-0-methyl-m7GpppG cap or an anti-reverse cap analog.
39. The immunogenic composition according to any one of items 15 to 38, wherein the nucleic acid as defined in i), ii) and/or iii) comprises a cap-independent translational enhancer.
40. A vaccine composition comprising the immunogenic composition as defined in any one of items 1 to 39 and a pharmaceutically acceptable carrier or excipient.
41. The vaccine composition according to item 40, wherein said immunogenic composition is encapsulated, attached to a carrier surface or is formulated for delivery to said subject by electroporation.
42. The vaccine composition according to item 41, wherein said immunogenic composition is encapsulated, such as encapsulated in a particle.
43. The vaccine composition according to item 42, wherein said particle is made of at least one or more of components selected from the group consisting of a lipid, a polymer, cholesterol and/or a cell penetrating peptide.
44. The vaccine composition according to item 43, wherein said particle is a lipid particle and/or a liposome.
45. The vaccine composition according to any one of items 42 to 44, wherein said particle is a nanoparticle, such as a lipid nanoparticle.
46. The vaccine composition according to any one of items 40 to 45, wherein the vaccine composition is formulated for intramuscular administration, subcutaneous administration, intradermal administration, intranasal administration, intravaginal administration, intrarectal administration or topical administration.
47. The vaccine composition according to item 46, wherein the vaccine composition is formulated for intramuscular administration or subcutaneous administration.
48. The vaccine composition according to item 46 or 47, wherein the vaccine composition is formulated for intramuscular administration.
49. The vaccine composition according to any one of items 40 to 48, wherein the vaccine composition is formulated for injection or for electroporation, such as wherein the vaccine composition is formulated for injection.
50. The immunogenic composition according to any one of items 1 to 39 or the vaccine composition according to any one of items 40 to 49, wherein the immunogenic composition or the vaccine composition elicits an HSV-2-specific antibody response, such as a serum IgG response, in said subject.
51. The immunogenic composition or the vaccine composition according to item 50, wherein said serum IgG response is detectable in a serum sample obtained from said subject at a dilution of at least about 10³, such as at least about 10⁵, such as at least about 10⁶, optionally as measured by ELISA.
52. The immunogenic composition or the vaccine composition according to item 50 or 51, wherein said serum IgG response is measured in comparison to serum IgG levels detectable in a control serum sample.
53. The immunogenic composition according to any one of items 1 to 39 and 50 to 52 or the vaccine composition according to any one of items 40 to 52, wherein the immunogenic composition or the vaccine composition induces an HSV-2 antigen-specific T cell response upon restimulation of said subject with one or more HSV-2-specific antigens.
54. The immunogenic composition or the vaccine composition according to item 53, wherein said HSV-2 antigen-specific T cell response comprises inducing an increase in the number of INF-γ, IL-2 and/or TNF-α secreting HSV-2 antigen-specific T cells.
55. The immunogenic composition or the vaccine composition according to item 53 or 54, wherein said HSV-2 antigen-specific T cell response comprises inducing an increase in the number of INF-γ and/or TNF-α secreting HSV-2 antigen-specific T cells.
56. The immunogenic composition or the vaccine composition according to any one of items 53 to 55, wherein said HSV-2 antigen-specific T cell response comprises inducing an increase in the number of INF-γ secreting HSV-2 antigen-specific T cells.
57. The immunogenic composition or the vaccine composition according to any one of items 53 to 56, wherein said increase in the number of INF-γ secreting HSV-2 antigen-specific T cells is at least about 2-fold, such as at least about 2.5-fold, such as at least about 5-fold, such as at least about 7.5-fold, such as at least about 10-fold, such as at least about 20-fold, such as at least about 25-fold, such as at least about 50-fold, such as at least about 75-fold, such as at least about 100-fold, such as at least about 200-fold, such as at least about 250-fold, such as at least about 500-fold, such as at least about 750-fold, such as at least about 1000-fold, such as at least about 1500-fold, such as at least about 2000-fold, such as at least about 2500-fold, such as at least about 3000-fold, such as at least about 3500-fold, such as at least about 4000-fold, such as at least about 5000-fold, such as at least about 6000-fold, such as at least about 7000-fold, as measured in comparison to a control sample.
58. The immunogenic composition or the vaccine composition according to any one of items 53 to 54, wherein said increase in the number of IL-2 secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, as measured in comparison to said control sample.
59. The immunogenic composition or the vaccine composition according to any one of items 53 to 55, wherein said increase in the number of TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, as measured in comparison to said control sample.
60. The immunogenic composition or the vaccine composition according to any one of items 53 to 56, wherein said increase in the number of INF-γ secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, such as at least about 25-fold, such as at least about 30-fold, such as at least about 35-fold, such as at least about 40-fold, such as at least about 50-fold, such as at least about 60-fold, such as at least about 70-fold, as measured in comparison to a control sample obtained from a subject administered UL11, UL16 and UL21 proteins, such as administered a multimeric protein complex comprising UL11, UL16 and UL21 proteins.
61. The immunogenic composition or the vaccine composition according to any one of items 53 to 54, wherein said increase in the number of IL-2 secreting HSV-2 antigen-specific T cells is at least about 1.5-fold, such as at least about 2-fold, such as at least about 2.5-fold, such as at least about 3-fold, as measured in comparison to said control sample obtained from a subject administered UL11, UL16 and UL21 proteins, such as administered a multimeric protein complex comprising UL11, UL16 and UL21 proteins.
62. The immunogenic composition or the vaccine composition according to any one of items 53 to 55, wherein said increase in the number of TNF-α secreting HSV-2 antigen-specific T cells is at least about 1.5-fold, such as at least about 2-fold, such as at least about 2.5-fold, such as at least about 3-fold, such as at least about 3.5-fold, such as at least about 4-fold, as measured in comparison to said control sample obtained from a subject administered UL11, UL16 and UL21 proteins, such as administered a multimeric protein complex comprising UL11, UL16 and UL21 proteins.
63. The immunogenic composition or the vaccine composition according to any one of items 53 to 62, wherein said HSV-2 antigen-specific T cell response comprises inducing multifunctional HSV-2 antigen-specific T cells, such as wherein said HSV-2 antigen-specific T cell response comprises inducing an increase in the number of multifunctional HSV-2 antigen-specific T cells.
64. The immunogenic composition or the vaccine composition according to item 63, wherein said multifunctional HSV-2 antigen-specific T cells are selected from a group consisting of INF-γ/IL-2, IL-2/TNF-α, INF-γ/TNF-α and INF-γ/IL-2/TNF-α secreting HSV-2 antigen-specific T cells.
65. The immunogenic composition or the vaccine composition according to item 63 or 64, wherein said multifunctional HSV-2 antigen-specific T cells are selected from a group consisting of INF-γ/IL-2, INF-γ/TNF-α and INF-γ/IL-2/TNF-α secreting HSV-2 antigen-specific T cells.
66. The immunogenic composition or the vaccine composition according to any one of items 63 to 65, wherein said multifunctional HSV-2 antigen-specific T cells are selected from a group consisting of INF-γ/IL-2 and INF-γ/IL-2/TNF-α secreting HSV-2 antigen-specific T cells.
67. The immunogenic composition or the vaccine composition according to any one of items 63 to 65, wherein said multifunctional HSV-2 antigen-specific T cells are selected from a group consisting of INF-γ/TNF-α and INF-γ/IL-2/TNF-α secreting HSV-2 antigen-specific T cells.
68. The immunogenic composition or the vaccine composition according to any one of items 63 to 67, wherein said multifunctional HSV-2 antigen-specific T cells are INF-γ/IL-2/TNF-α secreting HSV-2 antigen-specific T cells.
69. The immunogenic composition or the vaccine composition according to item 63 or 64, wherein said multifunctional HSV-2 antigen-specific T cells are IL-2/TNF-α secreting HSV-2 antigen-specific T cells.
70. The immunogenic composition or the vaccine composition according to any one of items 63 to 66, wherein said multifunctional HSV-2 antigen-specific T cells are INF-γ/IL-2 secreting HSV-2 antigen-specific T cells.
71. The immunogenic composition or the vaccine composition according to any one of items 63 to 65 and 67, wherein said multifunctional HSV-2 antigen-specific T cells are INF-γ/TNF-α secreting HSV-2 antigen-specific T cells.
72. The immunogenic composition or the vaccine composition according to any one of items 64 to 66 and 70, wherein said increase in the number of INF-γ/IL-2 secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 100-fold, such as at least about 150-fold, such as at least about 200-fold, such as at least about 250-fold, such as at least about 300-fold, such as at least about 350-fold, such as at least about 400-fold, such as at least about 450-fold, such as at least about 500-fold, as measured in comparison to said control sample.
73. The immunogenic composition or the vaccine composition according to any one of items 64, 65, 67 and 71, wherein said increase in the number of INF-γ/TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 100-fold, such as at least about 150-fold, such as at least about 200-fold, such as at least about 250-fold, such as at least about 300-fold, such as at least about 350-fold, such as at least about 400-fold, such as at least about 450-fold, such as 500-fold, such as at least about 550-fold, such as at least about 600-fold, such as at least about 650-fold, such as at least about 700-fold, such as at least about 750-fold, such as at least about 800-fold, such as at least about 850-fold, such as at least about 900-fold, such as at least about 950-fold, such as at least about 1000-fold, such as at least about 1100-fold, such as at least about 1200-fold, such as at least about 1300-fold, such as at least about 1400-fold, such as at least about 1500-fold, such as at least about 1600-fold, such as at least about 1700-fold, as measured in comparison to said control sample.
74. The immunogenic composition or the vaccine composition according to item 64 or 69, wherein said increase in the number of IL-2/TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 15-fold, such as at least about 20-fold, as measured in comparison to said control sample.
75. The immunogenic composition or the vaccine composition according to any one of items 64 to 68, wherein said increase in the number of INF-γ/IL-2/TNF-α secreting HSV-2 antigen-specific T cells is at least about 10-fold, such as at least about 20-fold, such as at least about 25-fold, such as at least about 50-fold, such as at least about 75-fold, such as at least about 100-fold, such as at least about 125-fold, such as at least about 150-fold, such as at least about 175-fold, such as at least about 200-fold, such as at least about 300-fold, such as at least about 400-fold, such as at least about 450-fold, such as at least about 500-fold, as measured in comparison to said control sample.
76. The immunogenic composition or the vaccine composition according to any one of items 64 to 66 and 70, wherein said increase in the number of INF-γ/IL-2 secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 9-fold, such as at least about 10-fold, such as at least about 11-fold, such as at least about 12-fold, such as at least about 13-fold, such as at least about 14-fold, such as at least about 15-fold, such as at least about 16-fold, such as at least about 17-fold, such as at least about 18-fold, such as at least about 19-fold, such as at least about 20-fold, such as at least about 25-fold, such as at least about 30-fold, such as at least about 40-fold, such as at least about 50-fold, as measured in comparison to said control sample obtainable from a subject administered UL11, UL16 and UL21 proteins, such as administered a multimeric protein complex comprising UL11, UL16 and UL21 proteins.
77. The immunogenic composition or the vaccine composition according to any one of items 64, 65, 67 and 71, wherein said increase in the number of INF-γ/TNF-α secreting HSV-2 antigen-specific T cells is at least about 10-fold, such as at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 55-fold, such as at least about 60-fold, such as at least about 65-fold, such as at least about 70-fold, such as at least about 75-fold, such as at least about 80-fold, such as at least about 100-fold, such as at least about 150-fold, as measured in comparison to said control sample obtainable from a subject administered UL11, UL16 and UL21 proteins, such as administered a multimeric protein complex comprising UL11, UL16 and UL21 proteins.
78. The immunogenic composition or the vaccine composition according to any one of items 64 to 68, wherein said increase in the number of INF-γ/IL-2/TNF-α secreting HSV-2 antigen-specific T cells is at least about 2-fold, such as at least about 3-fold, such as at least about 4-fold, such as at least about 5-fold, such as at least about 6-fold, such as at least about 7-fold, such as at least about 8-fold, such as at least about 10-fold, such as at least about 15-fold, as measured in comparison to said control sample obtainable from a subject administered UL11, UL16 and UL21 proteins, such as administered a multimeric protein complex comprising UL11, UL16 and UL21 proteins.
79. The immunogenic composition or the vaccine composition according to any one of items 53 to 78, wherein said HSV-2 antigen-specific T cell response is measured by a Fluorospot analysis.
80. A method for therapeutic treatment and/or prophylactic treatment of an HSV-2 infection comprising administration of the immunogenic composition according to any one of items 1 to 39 and 50 to 79 or the vaccine composition according to any one of items 40 to 79 to a subject, such as a human subject, in need thereof.
81. The method for therapeutic treatment and/or prophylactic treatment according to item 80, wherein said therapeutic treatment comprises ameliorating and/or eliminating symptoms associated with said HSV-2 infection.
82. The method for therapeutic treatment and/or prophylactic treatment according to item 80, wherein said prophylactic treatment comprises preventing symptoms associated with said HSV-2 infection.
83. The method for therapeutic treatment and/or prophylactic treatment according to any one of items 80 to 82, wherein said HSV-2 infection is selected from a group consisting of a primary HSV-2 infection and a reactivation of a latent HSV-2 infection, such as wherein said HSV-2 infection is a reactivation of a latent HSV-2 infection.
84. The method for therapeutic treatment and/or prophylactic treatment according to item 83, wherein said reactivation of a latent HSV-2 infection is a flare, a recurrence and/or an HSV-2 labialis following a primary HSV-2 infection.
85. The method for therapeutic treatment and/or prophylactic treatment according to any one of items 80 to 84, wherein said HSV-2 infection is a genital HSV-2 infection and/or an oral HSV-2 infection.
86. The method for therapeutic treatment and/or prophylactic treatment according to any one of items 80 to 85, wherein said HSV-2 infection causes HSV-2 encephalitis.
87. The method for therapeutic treatment and/or prophylactic treatment according to any one of items 80 to 87, wherein said HSV-2 infection is a neonatal infection.
88. The method for therapeutic treatment and/or prophylactic treatment according to any one of items 80 to 87, wherein said prophylactic treatment comprises preventing a reactivation of a latent HSV-2 infection, such as preventing a flare, a recurrence and/or an HSV-2 labialis following a primary HSV-2 infection.
89. The method for therapeutic treatment and/or prophylactic treatment according to any one of items 80 to 88, wherein said method comprises administration of the immunogenic composition or the vaccine composition in a single dose to said subject, wherein said single dose corresponds to the total amount of nucleic acids as defined in i), ii) and iii) and is less than about 200 μg, such as less than about 150 μg, such as less than about 100 μg, such as is between about 10 μg and about 100 μg.
90. The method for therapeutic treatment and/or prophylactic treatment according to any one of items 80 to 89, wherein said method comprises multiple administration of the immunogenic composition or the vaccine composition to said subject.
91. The method for therapeutic treatment and/or prophylactic treatment according to item 90, wherein said multiple administration comprises a prime vaccination and at least one boost vaccination, such as wherein said multiple administration is a two-dose prime-boost regimen comprising said prime vaccination and one of said at least one boost vaccination.
92. The method for therapeutic treatment and/or prophylactic treatment according to item 90, wherein said two-dose prime-boost regimen is administered repeatedly on an annual basis to said subject.
93. The method for therapeutic treatment and/or prophylactic treatment according to item 91 or 92, wherein the dose of said prime vaccination corresponds to the total amount of nucleic acids as defined in i), ii) and iii) and is less than about 200 μg, such as less than about 150 μg, such as less than about 100 μg, such as is between about 10 μg and about 100 μg.
94. The method for therapeutic treatment and/or prophylactic treatment according to any one of items 91 to 93, wherein the dose of said at least one boost vaccination corresponds to the total amount of nucleic acids as defined in i), ii) and iii) and is less than about 200 μg, such as less than about 150 μg, such as less than about 100 μg, such as is between about 10 μg and about 100 μg.
95. The method for therapeutic treatment and/or prophylactic treatment according to any one of items 91 to 94, wherein the time interval between said prime vaccination and said at least one boost vaccination is less than about 10 weeks, such as less than about 9 weeks, such as less than about 8 weeks, such as a time interval between about 2 weeks and about 8 weeks.

1. An immunogenic composition capable of eliciting a Herpes Simplex Virus 2 (HSV-2)-specific immune response in a subject when administered to said subject, wherein said immunogenic composition comprises at least one, such as at least two, such as all three nucleic acid(s) selected from the group consisting of:
(i) a nucleic acid encoding a UL11 protein of HSV-2 or an immunogenic fragment thereof,
(ii) a nucleic acid encoding a UL16 protein of HSV-2 or an immunogenic fragment thereof, and
(iii) a nucleic acid encoding a UL21 protein of HSV-2 or an immunogenic fragment thereof, wherein said UL11 protein comprises an amino acid sequence having at least 80% identity to SEQ ID NO:1, said UL16 protein comprises an amino acid sequence having at least 80% identity to SEQ ID NO:2, and said UL21 protein comprises an amino acid sequence having at least 80% identity to SEQ ID NO:3.

2. The immunogenic composition according to claim 1, wherein the composition does not comprise a viral envelope.

3. The immunogenic composition according to claim 1, wherein said immunogenic composition comprises all three nucleic acids as defined in (i), (ii) and (iii).

4. The immunogenic composition according to claim 1, wherein each nucleic acid as defined in i), ii) and/or iii) is present in an individual nucleic acid construct or wherein at least two of said nucleic acid as defined in i), ii) and/or iii) are present in the same nucleic acid construct, such as wherein all three of said nucleic acid as defined in i), ii) and/or iii) are present in the same nucleic acid construct.

5. The immunogenic composition according to claim 1, wherein each nucleic acid as defined in i), ii) and/or iii) is present in an individual nucleic acid construct.

6. The immunogenic composition according to claim 1, wherein the nucleic acid as defined in i), ii) and/or iii) is selected from the group consisting of DNA and RNA, such as wherein said DNA or RNA comprises at least one non-naturally occurring substitution modification and/or non-naturally occurring nucleoside modification.

7. The immunogenic composition according to claim 1, wherein the nucleic acid as defined in i), ii) and/or iii) is RNA, such as mRNA, such as codon-optimized mRNA, such as codon-optimized and nucleoside modified mRNA.

8. The immunogenic composition according to claim 7, wherein said nucleic acid as defined in i) is selected from the group consisting of a nucleic acid sequence that has at least 80% identity to SEQ ID NO:16 and contains at least one non-naturally occurring nucleoside modification relative to SEQ ID NO:16; said nucleic acid as defined in ii) is selected from the group consisting of a nucleic acid sequence that has at least 80% identity to SEQ ID NO:17 and contains at least one non-naturally occurring nucleoside modification relative to SEQ ID NO:17; and said nucleic acid as defined in iii) is selected from the group consisting of a nucleic acid sequence that has at least 80% identity to SEQ ID NO: 18 and contains at least one non-naturally occurring nucleoside modification relative to SEQ ID NO:18.

9. The immunogenic composition according to claim 8, wherein the nucleic acid as defined in i) comprises a nucleic acid sequence selected from a group consisting of any codon-optimized version of SEQ ID NO:16; the nucleic acid as defined in ii) comprises a nucleic acid sequence selected from a group consisting of any codon-optimized version of SEQ ID NO:17; and wherein the nucleic acid as defined in iii) comprises a nucleic acid sequence selected from a group consisting of any codon-optimized version of SEQ ID NO:18.

10. The immunogenic composition according to claim 9, wherein the nucleic acid as defined in i) comprises a nucleic acid sequence according to SEQ ID NO:86; the nucleic acid as defined in ii) comprises a nucleic acid sequence according to SEQ ID NO:87; and the nucleic acid as defined in iii) comprises a nucleic acid sequence according to SEQ ID NO:88.

11. The immunogenic composition according to claim 1, wherein each nucleic acid as defined in i), ii) and/or iii) is present in an individual nucleic acid construct or
wherein at least two of said nucleic acid as defined in i), ii) and/or iii) are present in the same nucleic acid construct, such as wherein all three of said nucleic acid as defined in i), ii) and/or iii) are present in the same nucleic acid construct.

12. The immunogenic composition according to claim 1, wherein the nucleic acid as defined in i), ii) and/or iii) is a nucleoside modified mRNA comprising one or more modified nucleosides, such as a nucleoside modified mRNA comprising one or more non-naturally occurring modified nucleosides, such as a nucleoside modified mRNA comprising one or more pseudouridine residues.

13. A vaccine composition comprising the immunogenic composition as defined in claim 1 and a pharmaceutically acceptable carrier or excipient.

14. The vaccine composition according to claim 13, wherein said immunogenic composition is encapsulated, attached to a carrier surface or is formulated for delivery to said subject by electroporation.

15. The vaccine composition according to claim 14, wherein said immunogenic composition is encapsulated in a particle.

16. The vaccine composition according to claim 15, wherein said immunogenic composition is encapsulated in a lipid nanoparticle.

17. The vaccine composition according to claim 13, wherein the vaccine composition is formulated for intramuscular administration or subcutaneous administration.

18. A method for therapeutic treatment and/or prophylactic treatment of an HSV-2 infection comprising administration of the immunogenic composition according to claim 1 or the vaccine composition comprising the immunogenic composition to a subject in need thereof.

19. The method for therapeutic treatment and/or prophylactic treatment according to claim 18, wherein said HSV-2 infection is selected from a group consisting of a primary HSV-2 infection and a reactivation of a latent HSV-2 infection, such as wherein said HSV-2 infection is a reactivation of a latent HSV-2 infection.

20. The method for therapeutic treatment and/or prophylactic treatment according to claim 19, wherein said HSV-2 infection selected from the group consisting of is a genital HSV-2 infection, an oral HSV-2 infection, an HSV-2 infection causing HSV-2 encephalitis and a neonatal HSV-2 infection.


SEQUENCE LISTING (88 sequences)

SEQ ID NO: 1 - protein, 96 aa, Human alphaherpesvirus 2
MGLAFSGARPCCCRHNVIITDGGEVVSLTAHEFDVVDIESEEEGNFYVPPDMRVVTRAPGPQYRRASDPPSRHTRRRDPDVARPPATLTPPLSDSE

SEQ ID NO: 2 - protein, 372 aa, Human alphaherpesvirus 2
MAQRALWRPQATPGPPGAAAPPGHRGAPPDARAPDPGPEADLVARIANSVFVWRVVRGDERLKIFRCLTVLTEPLCQVALPDPDPERALFCEIFLYLTRPKALRLPSNTFFAIFFFNRERRYCATVHLRSVTHPRTPLLCTLAFGHLEAASPPEETPDPAAEQLADEPVAHELDGAYLVPTEPPPNPGACCALGPGAWWHLPGGRIYCWAMDDDLGSLCPPGSRARHLGWLLSRITDPPGGGGACAPTAHIDSANALWRAPAVAEACPCVAPCMWSNMAQRTLAVRGDASLCQLLFGHPVDAVILRQATRRPRITAHLHEVVVGRDGAESVIRPTSAGWRLCVLSSYTSRLFATSCPAVARAVARASSSDYK

SEQ ID NO: 3 - protein, 532 aa, Human alphaherpesvirus 2
MELSYATTLHHRDVVFYVTADRNRAYFVCGGSVYSVGRPRDSQPGEIAKFGLVVRGTGPKDRMVANYVRSELRQRGLRDVRPVGEDEVFLDSVCLLNPNVSSERDVINTNDVEVLDECLAEYCTSLRTSPGVLVTGVRVRARDRVIELFEHPAIVNISSRFAYTPSPYVFALAQAHLPRLPSSLEPLVSGLFDGIPAPRQPLDARDRRTDVVITGTRAPRPMAGTGAGGAGAKRATVSEFVQVKHIDRVVSPSVSSAPPPSAPDASLPPPGLQEAAPPGPPLRELWWVFYAGDRALEEPHAESGLTREEVRAVHGFREQAWKLFGSVGAPRAFLGAALALSPTQKLAVYYYLIHRERRMSPFPALVRLVGRYIQRHGLYVPAPDEPTLADAMNGLFRDALAAGTVAEQLLMFDLLPPKDVPVGSDARADSAALLRFVDSQRLTPGGSVSPEHVMYLGAFLGVLYAGHGRLAAATHTARLTGVTSLVLTVGDVDRMSAFDRGPAGAAGRTRTAGYLDALLTVCLARAQHGQSV

SEQ ID NO: 4 - protein, 548 aa, Human alphaherpesvirus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 5 - protein, 106 aa, synthetic construct
  REGION 1..106: note = Cytoplasmic tail of gE protein of HSV-2 (human herpesvirus 2)
RRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 6 - protein, 490 aa, Human alphaherpesvirus 2
MDLLVDDLFADADGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 7 - protein, 302 aa, Human alphaherpesvirus 2
MTSRRSVKSCPREAPRGTHEELYYGPVSPADPESPRDDFRRGAGPMRARPRGEVRFLHYDEAGYALYRDSSSSEDNDESRDTARPRRSASVAGSHGPGPARAPPPPGGPVGAGGRSHAPPARTPKMTRGAPKAPATPATDPARGRRPAQADSAVLLDAPAPTASGRTKTPAQGLAKKLHFSTAPPSPTAPWTPRVAGFNKRVFCAAVGRLAATHARLAAVQLWDMSRPHTDEDLNELLDLTTIRVTVCEGKNLLQRANELVNPDAAQDVDATAAARGRPAGRAAATARAPARSASRPRRPLE

SEQ ID NO: 8 - protein, 305 aa, Human alphaherpesvirus 2
MYDIAPRRSGSRPGPGRDKTRRRSRFSAAGNPGVERRASRKSLPSHARRLELCLHERRRYRGFFAALAQTPSEEIAIVRSLSVPLVKTTPVSLPFSLDQTVADNCLTLSGMGYYLGIGGCCPACSAGDGRLATVSREALILAFVQQINTIFEHRTFLASLVVLADRHSTPLQDLLADTLGQPELFFVHTILRGGGACDPRFLFYPDPTYGGHMLYVIFPGTSAHLHYRLIDRMLTACPGYRFAAHVWQSTFVLVVRRNAEKPADAEIPTVSAADIYCKMRDISFDGGLMLEYQRLYATFDEFPPP

SEQ ID NO: 9 - protein, 250 aa, Human alphaherpesvirus 2
MAGMGKPYGGRPGDAFEGLVQRIRLIVPTTLRGGGGESGPYSPSNPPSRCAFQFHGQDGSDEAFPIEYVLRLMNDWADVPCNPYLRVQNTGVSVLFQGFFNRPHGAPGGAITAEQTNVILHSTETTGLSLGDLDDVKGRLGLDARPMMASMWISCFVRMPRVQLAFRFMGPEDAVRTRRILCRAAEQALARRRRSRRSQDDYGAVAVAAAHHSSGAPGPGVAASGPPAPPGRGPARPWHQAVQLFRAPRP

SEQ ID NO: 10 - protein, 904 aa, Human alphaherpesvirus 2
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 11 - protein, 393 aa, Human alphaherpesvirus 2
MGRLTSGVGTAALLVVAVGLRVVCAKYALADPSLKMADPNRFRGKNLPVLDRLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 12 - RNA, 1386 nt, synthetic construct
  misc_feature 1..1386: note = All uridine residues in SEQ ID NO:12 are 1-methyl-pseudouridine (m1f)
  source 1..1386: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgacccgcctgaccgtgctggccctgctggccggcctgctggcctcctcccgcgccaagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 13 - RNA, 1614 nt, synthetic construct
  misc_feature 1..1614: note = All uridine residues in SEQ ID NO:13 are 1-methyl-pseudouridine (m1f)
  source 1..1614: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 14 - RNA, 1470 nt, synthetic construct
  misc_feature 1..1470: note = UL48 of HSV-2 RNA sequence
  source 1..1470: mol_type = other RNA
atggatctgctggtggatgatctgtttgcggatgcggatggcgtgagcccgccgccgccgcgcccggcgggcggcccgaaaaacaccccggcggcgccgccgctgtatgcgaccggccgcctgagccaggcgcagctgatgccgagcccgccgatgccggtgccgccggcggcgctgtttaaccgcctgctggatgatctgggctttagcgcgggcccggcgctgtgcaccatgctggatacctggaacgaagatctgtttagcggctttccgaccaacgcggatatgtatcgcgaatgcaaatttctgagcaccctgccgagcgatgtgattgattggggcgatgcgcatgtgccggaacgcagcccgattgatattcgcgcgcatggcgatgtggcgtttccgaccctgccggcgacccgcgatgaactgccgagctattatgaagcgatggcgcagttttttcgcggcgaactgcgcgcgcgcgaagaaagctatcgcaccgtgctggcgaacttttgcagcgcgctgtatcgctatctgcgcgcgagcgtgcgccagctgcatcgccaggcgcatatgcgcggccgcaaccgcgatctgcgcgaaatgctgcgcaccaccattgcggatcgctattatcgcgaaaccgcgcgcctggcgcgcgtgctgtttctgcatctgtatctgtttctgagccgcgaaattctgtgggcggcgtatgcggaacagatgatgcgcccggatctgtttgatggcctgtgctgcgatctggaaagctggcgccagctggcgtgcctgtttcagccgctgatgtttattaacggcagcctgaccgtgcgcggcgtgccggtggaagcgcgccgcctgcgcgaactgaaccatattcgcgaacatctgaacctgccgctggtgcgcagcgcggcggcggaagaaccgggcgcgccgctgaccaccccgccggtgctgcagggcaaccaggcgcgcagcagcggctattttatgctgctgattcgcgcgaaactggatagctatagcagcgtggcgaccagcgaaggcgaaagcgtgatgcgcgaacatgcgtatagccgcggccgcacccgcaacaactatggcagcaccattgaaggcctgctggatctgccggatgatgatgatgcgccggcggaagcgggcctggtggcgccgcgcatgagctttctgagcgcgggccagcgcccgcgccgcctgagcaccaccgcgccgattaccgatgtgagcctgggcgatgaactgcgcctggatggcgaagaagtggatatgaccccggcggatgcgctggatgattttgatctggaaatgctgggcgatgtggaaagcccgagcccgggcatgacccatgatccggtgagctatggcgcgctggatgtggatgattttgaatttgaacagatgtttaccgatgcgatgggcattgatgattttggcggc

SEQ ID NO: 15 - RNA, 906 nt, synthetic construct
  misc_feature 1..906: note = UL49 of HSV-2 RNA sequence
  source 1..906: mol_type = other RNA
atgacctctcgccgctccgtcaagtcgtgtccgcgggaagcgccgcgcgggacccacgaggagctgtactatggcccggtctccccggcggatccagagagtccgcgcgacgacttccgccgcggcgctggcccgatgcgcgcgcgcccaaggggcgaggttcgctttctccattatgacgaggctgggtatgccctctaccgggactcgtcgtcgtccgaagacaacgacgagtcccgggataccgcgcgaccgcgtcgttcggcgtccgtcgcgggctctcacggccccggccccgcgcgcgctcctccaccccccgggggccccgtgggcgccggcgggcgctcgcacgcccctcccgcgcggacccccaaaatgacgcgcggggcgcctaaggcccccgcgaccccggcgaccgaccctgcccgcggcaggcgacccgcccaggccgactccgccgtgctcctagacgcccccgctcccacggcctcgggaagaaccaagacacccgcccagggactggccaagaagctgcacttcagcaccgccccaccgagccccacggcgccgtggaccccccgggtggccgggttcaacaagcgcgtcttctgcgccgcggtcgggcgcctggcggccacgcacgcccggctggcggcggtacagctgtgggacatgtcgcggccgcacaccgacgaagacctcaacgagctcctcgacctcaccaccattcgcgtgacggtctgcgagggcaagaacctcctgcagcgcgcgaacgagttggtgaatcccgacgcggcgcaggacgtcgacgcgaccgcggccgcccggggccgccccgcggggcgtgccgccgcgaccgcacgggcccccgcccgctcggcttcccgtccccgccgccccctcgag

SEQ ID NO: 16 - RNA, 291 nt, synthetic construct
  misc_feature 1..291: note = UL11 of HSV-2 RNA sequence
  source 1..291: mol_type = other RNA
atgggcctcgcgttctccggggcgcggccctgctgctgccgacacaacgtcatcatcaccgacggcggggaggtcgtctcgctgaccgcccacgaatttgacgtcgtggacatcgagtccgaagaagagggtaacttctacgtgccccccgacatgcgcgtggtcacccgggcgccggggccccagtaccggcgcgcatcggaccccccatcgcgtcacaccagacggcgggaccccgacgtggcccgccctcctgccacgctcacgcccccactctctgacagcgaataa

SEQ ID NO: 17 - RNA, 1119 nt, synthetic construct
  misc_feature 1..1119: note = UL16 of HSV-2 RNA sequen
  source 1..1119: mol_type = other RNA
atggcacagcgggcactctggcgtccccaggcgacgccaggccccccgggagccgcggccccgccgggtcaccggggcgccccccccgacgcgcgcgcccccgacccggggcccgaagccgacctcgtcgcgcgcatcgccaactctgtcttcgtgtggcgcgtcgttcggggggacgagcggcttaagatttttcggtgcctcacggtcctcaccgagccgctgtgccaggtggcccttccggaccccgaccccgagcgtgcccttttctgcgagatcttcctgtacctgacgcggcccaaggcgctgcgcctgccttcgaacacgttctttgccattttcttctttaaccgcgagcgccgctactgcgccaccgtccacctccgcagcgtaacgcacccccggaccccgctgctgtgcaccctagccttcggccacctggaagctgcctcccccccggaggaaacccccgaccccgccgccgaacagctcgcggacgagcccgtggcccacgagctcgacggcgcctacctggtacccacggaaccgcccccgaatccgggggcgtgctgtgccctgggtccgggggcctggtggcacctccccggcggccgcatatactgctgggccatggatgatgacctggggtcgctctgtcccccgggcagccgggcccgccatttaggatggctgctatccaggattaccgaccccccgggaggaggcggcgcctgcgccccgacggcccacatcgattccgccaacgcgctgtggcgcgcccccgccgtggcggaggcctgtccctgcgtcgccccgtgcatgtggtccaatatggcccagcgcaccctggccgtccggggggacgcgagcctgtgtcagctcctcttcgggcaccccgtggacgcggttatcctgcggcaggcgacccgccgcccgcgcatcacggcccacctgcacgaggtcgtcgtgggccgggacggcgcggaaagcgtcatccgcccgaccagcgccgggtggcgcctctgcgttctgtcatcgtataccagccgcctgtttgcaacgagctgccccgcggtcgcgcgggccgtggccagagcctcatcgtccgattacaaataa

SEQ ID NO: 18 - RNA, 1599 nt, synthetic construct
  misc_feature 1..1599: note = UL21 of HSV-2 RNA sequence
  source 1..1599: mol_type = other RNA
atggagctcagctatgccaccaccctgcaccaccgggacgttgtgttttacgtcacggcagacagaaaccgcgcctactttgtgtgcggggggtccgtttattccgtagggcggcctcgggattctcagccgggggaaattgccaagtttggcctggtggtccgggggacaggccccaaagaccgcatggtcgccaactacgtacgaagcgagctccgccagcgcggcctgcgggacgtgcggcccgtgggggaggacgaggtgttcctggacagcgtgtgtctgctaaacccgaacgtgagctccgagcgagacgtgattaataccaacgacgttgaagtgctggacgaatgcctggccgaatactgcacctcgctgcgaaccagcccgggggtgctggtgaccggggtgcgcgtgcgcgcgcgagacagggtcatcgagctatttgagcacccggcgatcgtcaacatttcctcgcgcttcgcgtacaccccctccccctacgtattcgccctggcccaggcgcacctcccccggctcccgagctcgctggagcccctggtgagcggcctgtttgacggcattcccgccccgcgccagcccctggacgcccgcgaccggcgcacggatgtcgtgatcacgggcacccgcgcccccagaccgatggccgggaccggggccgggggcgcgggggccaagcgggccaccgtcagcgagttcgtgcaagtgaagcacatcgaccgtgttgtgtccccgagcgtctcttccgcccccccgccgagcgcccccgacgcgagtctgccgcccccggggctccaggaggccgccccgccgggccccccgctcagggagctgtggtgggtgttctacgccggcgaccgggcgctggaggagccccacgccgagtcgggattgacgcgcgaggaggtccgcgccgtgcatgggttccgggagcaggcgtggaagctgtttgggtcggtgggggctccgcgggcgtttctcggggccgcgctggccctgagcccgacccaaaagctcgccgtctactactatctcatccaccgggagcggcgcatgtcccccttccccgcgctcgtgcggctcgtcggtcggtacatccagcgccacggcctgtacgttcccgcgcccgacgaaccgacgttggccgatgccatgaacgggctgttccgcgacgcgctggcggccgggaccgtggccgagcagctcctcatgttcgacctcctcccgcccaaggacgtgccggtggggagcgacgcgcgggccgacagcgccgccctgctgcgctttgtggactcgcaacgcctgaccccgggggggtccgtctcgcccgagcacgtcatgtacctcggcgcgttcctgggcgtgttgtacgccggccacggacgcctggccgcggccacgcataccgcgcgcctgacgggcgtgacgtccctggtcctgaccgtgggggacgtcgaccggatgtccgcgtttgaccgcgggccggcgggggcggctggccgcacgcgaaccgccgggtacctggacgcgctgcttaccgtttgcctggctcgcgcccagcacggccagtctgtgtga

SEQ ID NO: 19 - RNA, 918 nt, synthetic construct
  misc_feature 1..918: note = UL31 of HSV-2 RNA sequence
  source 1..918: mol_type = other RNA
atgtatgacatcgccccccgtcgctccggctcccggcccgggcccggccgcgacaagacccggcggcggtcgcgcttttccgccgccgggaaccccggcgtggagcgccgggcctcccgcaagagcctgccgtcgcacgcgcgcaggctggagctctgcctgcacgagcgccgtcgctaccgcggcttcttcgccgccctcgcccagacgccctccgaggagatcgccatcgtgcgctcgctctccgtgcccctggtgaagacgacccccgtgtcgctccccttcagcctggaccagaccgtggccgacaactgcctgaccctctcgggcatgggctactacctggggatcgggggctgctgccccgcgtgcagcgcgggcgacgggcggctggccaccgtcagccgcgaggccctcatcctggccttcgtgcagcagatcaatacgatctttgagcaccgcacctttctggcctccctggtcgtgctggccgaccgccacagcacccccctccaggacctcctggccgatacgctcggccagcccgagctctttttcgtgcacacgatcctgcgcggagggggggcgtgcgacccccggtttcttttttacccggaccccacgtacgggggccacatgctgtacgtcatcttccccggcacgtcggcccacctgcactaccggctcatcgatcggatgctgaccgcgtgccccggctaccggttcgccgcccacgtgtggcagagcacgtttgtgctcgtggtcagacgcaacgcagagaaaccagcggacgcggaaattccgaccgtgtcggccgcagacatttattgcaagatgcgggacatcagcttcgacggggggctcatgctagagtatcaaaggctctatgcaacattcgacgagtttcctccgccgtag

SEQ ID NO: 20 - RNA, 753 nt, synthetic construct
  misc_feature 1..753: note = UL34 of HSV-2 RNA sequence
  source 1..753: mol_type = other RNA
atggcggggatggggaagccctacggcggccgcccgggggacgcgttcgagggtctcgttcagcgcatcaggctcattgttcccaccacgctgcgcggcgggggtggggagtcgggcccctactcgccatccaacccgccctcgagatgtgccttccagttccacggccaggatgggtccgacgaggccttcccgatcgagtacgtcctgcggctcatgaacgactgggccgatgtgccctgcaacccctacctgcgcgtgcagaacaccggcgtttcggtgctgtttcaggggttttttaaccggccccacggcgccccggggggcgcgatcacggcggagcagaccaacgtgattctgcactccaccgagacgacgggactgtccctcggagacctggacgacgtcaaggggcgcctcggcctggacgcccggccgatgatggccagcatgtggatcagctgctttgtgcgcatgccccgggtgcagctcgcgtttcggttcatgggccccgaggacgccgttcgcacgcggcggatcctgtgtcgcgccgccgagcaggccctcgcccgtcgccgccggtccaggcggtcccaggatgactacggggcggtggcggtggcggcggcgcaccactcttccggagcgcccgggccgggggtcgccgcctcgggcccgccagcgccgcccggacggggaccggcccgtccgtggcatcaggccgtgcagttgttccgggccccgcgtccgtga

SEQ ID NO: 21 - RNA, 321 nt, synthetic construct
  misc_feature 1..321: note = Cytoplasmic tail of gE protein of HSV-2 RNA sequence
  source 1..321: mol_type = other RNA
cgcaggcgctcctggcgggcggttaaaagccgggcctcggcgacgggccccacttacattcgcgtggcggacagcgagctgtacgcggactggagttcggacagcgagggggagcgcgacgggtccctgtggcaggaccctccggagagacccgactctccctccacaaatggatccggctttgagatcttatcaccaacggctccgtctgtatacccccatagcgaggggcgtaaatctcgccgcccgctcaccacctttggttcgggaagcccgggccgtcgtcactcccaggcctcctattcgtccgtcctctggtaa

SEQ ID NO: 22 - RNA, 1182 nt, synthetic construct
  misc_feature 1..1182: note = gD of HSV-2 RNA sequence
  source 1..1182: mol_type = other RNA
atggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgttaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggcgctggtcatcggcggtattgcgttttgggtacgccgccgcgctcagatggcccccaagcgcctacgtctcccccacatccgggatgacgacgcgcccccctcgcaccagccattgttttactag

SEQ ID NO: 23 - RNA, 2192 nt, synthetic construct
  misc_feature 1..2192: note = gB of HSV-2 RNA sequence
  source 1..2192: mol_type = other RNA
atgcgcggtggcggattgatctgcgccctggtggtgggtgcactggtggctgctgtcgcttcagctgctcctgctgctcctgcagctcctcgcgctagtggtggcgttgctgccaccgtggcagcgaacggaggtccagctagccgtcctcccccagtcccgtctcctgcaaccactaaggcacgcaagcgtaaaaccaagaaaccgcctaagaggcctgaagcaactcccccaccggacgcaaacgctactgtggctgctggtcacgctactctcagggctcatttgagagagatcaaggttgaaaacgctgacgcccagttctacgtgtgccctcccccaacaggtgcaactgttgtgcagttcgaacagcctcgcaggtgtcctaccaggcctgagggtcaaaactacactgaaggcatcgctgtcgttttcaaggagaacattgccccatacaagttcaaagcaaccatgtactacaaagacgtgactgtctcgcaagtgtggttcggacaccgttactcccaattcatgggtatcttcgaggacagggctcccgttccattcgaggaagtgatcgataagattaacgccaaaggcgtgtgcaggtccacagcaaagtacgtcagaaacaacatggagacaacggctttccaccgcgacgatcatgagaccgacatggaattgaagcctgcgaaagtggctacaaggacgtcgagaggctggcacaccactgatctgaagtacaacccctccagggttgaagccttccatagatacggaacaacggtgaactgtatcgttgaggaagtggatgcccgctcagtgtacccctacgacgagttcgtcctggctaccggagatttcgtttacatgagtccattctacggatacagggagggttcgcacaccgaacatacttcctacgcagcggacagattcaagcaggtggatggtttctacgctcgcgacctgaccactaaagcacgtgcgacttctcctacaacgaggaacctgctcaccactcccaagttcacagtcgcttgggactgggttccaaagaggccggccgtctgcaccatgactaaatggcaggaggttgacgaaatgctgagagctgaatacggcggaagcttccgcttctccagcgatgccatctctacaacgttcaccactaacctgactcaatactccctcagccgtgtggacctgggtgactgtatcggacgcgatgcccgtgaggcaattgaccgcatgttcgcgcgtaagtacaacgctacacacatcaaagtgggccagccacaatactacctcgcgacgggtggcttcttgattgcttaccagcctttgctgtcaaacactctcgctgaattgtacgtcagggagtacatgagagaacaagacaggaagccaagaaacgcgacaccggctcctctgagagaggctccgtctgctaacgcctcagtggaacgcatcaagacaacctcctccattgagttcgctcgtctccagttcacctacaaccacatccaacgtcatgtgaacgacatgctgggaaggattgcagtcgcgtggtgcgagctccagaaccacgaactgaccctctggaacgaggccaggaagctcaacccaaacgctatcgcctcagcaactgtcggcaggagagttagtgcgagaatgttgggagacgtcatggctgttagcacctgcgttcctgtggcccccgataacgtcattgttcaaaactctatgcgcgtcagttcgcgtccaggaacttgttactcccgcccgctcgttagcttccgttacgaggaccagggtccactgatcgaaggtcaactcggcgagaacaacgaattgcgcctgacacgtgatgctctggagccttgtacggtcggtcaccgccgttacttcattttcggaggtggctacgtgtacttcgaggaatacgcatacagtcaccagttgtcgcgcgctgacgtgaccactgtctccacattcatcgacttgaacattacgatgctggaggatcacgaattcgtgcccctgggtgtctacacacgtcatgaaatcaaggacagcggcctcttggattacaccgaggtccagaggagaaaccaactgcacgacctcagattcgctgacatcgatactgtgattcgcgcggatgctaacgctgccatgttcgctgg

SEQ ID NO: 24 - RNA, 1251 nt, synthetic construct
  misc_feature 1..1251: note = gE of HSV-2 RNA sequence
  source 1..1251: mol_type = other RNA
atggccaggggcgccggcctggtgttcttcgtgggcgtgtgggtggtgagctgcctggccgccgcccccaggaccagctggaagagggtgaccagcggcgaggacgtggtgctgctgcccgcccccgccggccccgaggagaggaccagggcccacaagctgctgtgggccgccgagcccctggacgcctgcggccccctgaggcccagctgggtggccctgtggccccccaggagggtgctggagaccgtggtggacgccgcctgcatgagggcccccgagcccctggccatcgcctacagcccccccttccccgccggcgacgagggcctgtacagcgagctggcctggagggacagggtggccgtggtgaacgagagcctggtgatctacggcgccctggagaccgacagcggcctgtacaccctgagcgtggtgggcctgagcgacgaggccaggcaggtggccagcgtggtgctggtggtggagcccgcccccgtgcccacccccacccccgacgactacgacgaggaggacgacgccggcgtgagcgagaggacccccgtgagcgtgcccccccccaccccccccaggaggccccccgtggccccccccacccaccccagggtgatccccgaggtgagccacgtgaggggcgtgaccgtgcacatggagacccccgaggccatcctgttcgcccccggcgagaccttcggcaccaacgtgagcatccacgccatcgcccacgacgacggcccctacgccatggacgtggtgtggatgaggttcgacgtgcccagcagctgcgccgagatgaggatctacgaggcctgcctgtaccacccccagctgcccgagtgcctgagccccgccgacgccccctgcgccgtgagcagctgggcctacaggctggccgtgaggagctacgccggctgcagcaggaccacccccccccccaggtgcttcgccgaggccaggatggagcccgtgcccggcctggcctggctggccagcaccgtgaacctggagttccagcacgccagcccccagcacgccggcctgtacctgtgcgtggtgtacgtggacgaccacatccacgcctggggccacatgaccatcagcaccgccgcccagtacaggaacgccgtggtggagcagcacctgccccagaggcagcccgagcccgtggagcccaccaggccccacgtgagggcccccccccccgcccccagcgccaggggccccctgagg

SEQ ID NO: 25 - RNA, 1692 nt, synthetic construct
  misc_feature 1..1692: note = Codon-optimized UL48 of HSV-2 RNA sequence including UTRs and polyA, all uridine residues are 1-methyl-pseudouridine (m1f)
  source 1..1692: mol_type = other RNA
taatacgactcactataaggactcactatttgttttcgcgcccagttgcaaaaagtgtcgtcaccatggacctgctcgttgacgatttgttcgctgacgcagatggtgtgagccctcccccaccgaggccagctggtggccctaagaacacccctgctgcccctcccctgtacgcaactggcagattgtcacaggcgcaactgatgcctagtccaccgatgcctgtgcctcccgcagctctcttcaacaggttgctggacgatttgggcttcagcgcaggacccgcgttgtgcacaatgctggatacgtggaacgaggacctgttctctggcttccccacaaacgcagacatgtaccgcgagtgtaaattcctgtctacgctcccatcagacgtcatcgattggggcgacgcccacgttcccgaaaggtctccaatcgacattagagcacatggagatgtcgcgttcccaaccctgccggctactcgcgacgagctcccatcatactacgaagcgatggctcagttcttcaggggagagttgcgcgcccgtgaagaaagttacagaacagtgctggctaacttctgctcggccctctaccgctacttgcgtgcatccgtcaggcagctgcacagacaagcgcatatgaggggtagaaaccgcgacctgcgcgaaatgctccgtaccactatcgctgataggtactacagagagactgctcgcctggcccgtgtcttgttcctgcacctctacttgttcctgtcacgcgaaattctctgggctgcctacgccgagcaaatgatgcgtccagatttgttcgacggcctgtgctgtgacctcgagtcgtggcgccagttggcttgtctgttccaaccattgatgttcatcaacggatccctgaccgttcgtggtgtgcctgtggaggcacgccgtctccgcgaattgaaccacattcgtgagcatctcaacttgcctctggttcgttctgcagctgctgaggaacctggagctcccctcacaacgccaccggtgttgcagggcaaccaagcccgctccagcggatacttcatgctcttgatccgtgctaagctggactcctactcttcagtggccacctccgagggtgaatcggtcatgcgcgaacacgcttactcacgtggcaggacaagaaacaactacggtagtacgattgaaggcctgctcgacctgcccgacgatgacgatgcaccagcagaggctggactggtggctcctcgcatgtccttcctcagcgccggtcagagaccgaggagactcagcaccactgcccccatcaccgatgtctctttgggtgacgagctccgtttggatggcgaggaagttgacatgactcctgctgatgccctcgacgatttcgacctggaaatgctcggagatgtcgagagtccttcgcccggtatgacacacgaccccgtttcctacggtgctctggatgtggacgatttcgagttcgaacaaatgttcacggacgcaatgggcatcgacgatttcggaggtggcgcgggatccggaggtggcggaagcggtggcggaggttctcaccaccatcatcaccatcaccattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - RNA, 607 nt, synthetic construct
  misc_feature 1..607: note = Codon-optimized UL11 of HSV-2 RNA sequence including UTRs and polyA, all uridine residues are 1-methyl-pseudouridine (m1f)
  source 1..607: mol_type = other RNA
taatacgactcactataaggactcactatttgttttcgcgcccagttgcaaaaagtgtcgtcaccatgggtttggcttttagtggagctaggccttgttgttgtaggcataatgtgattattactgatggtggcgaagtggtgtcactcactgctcatgagttcgatgttgttgatatagaaagtgaggaagaagggaatttctatgtcccgccagatatgcgggtagtgactagagcccccggcccacaatatagaagagccagcgatccaccctctcgacatactcgacgacgagatccagatgtagcacggcccccagcgaccttgaccccacccctgtccgattccgagtgagagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - RNA, 1435 nt, synthetic construct
  misc_feature 1..1435: note = Codon-optimized UL16 of HSV-2 RNA sequence including UTRs and polyA, all uridine residues are 1-methyl-pseudouridine (m1f)
  source 1..1435: mol_type = other RNA
taatacgactcactataaggactcactatttgttttcgcgcccagttgcaaaaagtgtcgtcaccatggctcaaagagccctgtggagaccacaagccacccctggaccacccggcgctgccgctccacctggacatagaggtgctcccccagatgctagagctcctgatccaggtccggaagcagatttggttgccagaattgcgaatagcgtgtttgtctggagagttgtgcgcggtgatgaaaggctgaaaatcttccgctgtctgactgtacttacggaacccttatgtcaagtcgcattacccgatcctgatcctgaaagagctctgttttgtgaaatctttctgtatctcacccgcccgaaagctctccggctcccgtctaataccttcttcgcgatcttcttcttcaatagagaaaggcggtattgtgcaacagtgcatcttcggtctgttacccatccgagaacaccactcctctgtacactggcttttggacatctcgaggcggcttctccacccgaagagactcctgatccagcagctgagcaactggcagatgaaccagttgcacatgaacttgatggggcatatctcgttccaactgagccaccaccaaacccaggtgcctgttgcgcgttgggaccaggagcttggtggcatctgccaggtgggcggatctattgttgggctatggacgacgatctcggtagtctgtgcccacccgggagtcgtgctagacacctcggctggcttctttcacgcatcacggatccacccggggggggcggggcatgtgctcccacagctcatattgacagtgctaatgccctttggcgagctccagcagttgctgaagcttgcccgtgtgtggctccttgtatgtggagcaacatggcgcaacggacacttgctgtgcgcggtgatgcctcactctgccaattgctgtttggtcatcctgtcgatgctgtgatacttcgtcaagccactagacgtcccaggattacagcacatctccatgaagtggtggtaggaagggatggagcagagagtgtgattaggcccacgtccgctggctggagactgtgtgtgttaagctcctacacaagtaggcttttcgctactagttgtccggccgttgcaagagctgtcgcaagggcatccagttctgactataagtaggagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - RNA, 1915 nt, synthetic construct
  misc_feature 1..1915: note = Codon-optimized UL21 of HSV-2 RNA sequence including UTRs and polyA, all uridine residues are 1-methyl-pseudouridine (m1f)
  source 1..1915: mol_type = other RNA
taatacgactcactataaggactcactatttgttttcgcgcccagttgcaaaaagtgtcgtcaccatggaactgtcctacgcaacaactcttcatcatagagatgtagtcttctatgtgaccgccgatcggaatagggcatatttcgtctgtggcggtagtgtctactctgtgggcagaccgagggacagccaacctggcgagatcgctaaattcgggctcgtcgtgaggggtaccggaccaaaggatagaatggtggcaaattatgtgcggtccgaattgaggcaaagaggtctccgcgatgttagacctgttggcgaagatgaagtatttctcgattccgtttgcttgttgaatccaaatgtttcctcagaacgcgatgtaatcaacactaatgatgtcgaggtcttggatgagtgtctcgctgagtattgtacaagcctcaggacgagtcccggtgttttagtcacaggagttagggtcagagcacgcgatagagtgattgaattattcgaacatcccgctattgtaaatatctctagccgatttgcatatacgccttctccatatgtgtttgctctcgctcaagcccatctgccacgtctgccatcctcactcgaaccgctcgtatctgggttgttcgatgggataccggcaccaagacaaccgctcgatgctcgagatcgccgtaccgatgtggttataaccgggacaagagcaccacggcctatggcgggaacaggcgcaggcggagccggcgctaaacgcgctacagtgtccgaatttgtacaggtcaaacatatagatagagtggtctcacctagtgtaagcagtgcgccaccccctagtgcgcctgatgcttcacttcccccgcccggactgcaagaagcggcaccaccaggaccacccctgagagaattatggtgggtcttttatgctggagatagggctcttgaagaacctcatgcagaaagcggcctgaccagggaagaagtgagggctgtccacggttttagagaacaagcctggaaattattcggctctgttggagcccccagggccttcctgggcgcagcattagcactctctcccacacagaaactggcagtgtattattacttgatacatcgcgaaaggagaatgtctccgtttccggcccttgtacgcttggtgggaagatatattcaaaggcatggactctatgtgcctgccccagatgagcccaccctcgctgacgctatgaatggtctgtttcgggatgctcttgctgcaggcacagttgcagaacaactgttaatgtttgatttgttaccccctaaagatgtaccagttggatctgatgcaagagctgatagtgctgcactcctcagattcgtcgatagccagcggctcacacccggtggcagcgtgagcccagaacatgtgatgtatctcggggcatttcttggtgttctttatgctgggcatggcagacttgctgccgcgactcacactgctcggttgacaggtgtaacaagcctcgttttgacagttggcgatgtagataggatgtcagccttcgatcgaggtccagccggcgcagcaggacgaactagaactgcaggatatctcgatgctctcttaacagtgtgtcttgcccgggctcaacatgggcaatccgtttaggagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 1339 nt, synthetic construct
  misc_feature 1..1339: note = Codon-optimized gD of HSV-2 RNA sequence including UTRs and polyA, all uridine residues are 1-methyl-pseudouridine (m1f)
  source 1..1339: mol_type = other RNA
taatacgactcactataaggactcactatttgttttcgcgcccagttgcaaaaagtgtcgtcaccatgggccgcctcactagtggggttggtactgctgcgttactggtcgtggctgtcgggcttagagtggtttgtgctaagtatgcacttgctgatccatcactgaaaatggcagacccaaaccgcttcaggggtaagaatttgcccgtgctggatcaattgacagatccaccgggcgttaaaagagtctatcatatccaacccagtctcgaagatccttttcaacccccttccattcccattaccgtatattatgccgtattagagcgggcttgtcgttccgtacttttgcacgcgccatccgaagcacctcaaattgtcagaggagcatccgatgaagctagaaaacatacctataatctcacaatagcttggtacagaatgggcgataactgtgccattccaattacagtcatggagtatacggaatgtccttataataaaagtctcggagtatgtccaattagaacccaaccacggtggtcatattacgattccttcagtgcggtttctgaagacaatctcgggtttctcatgcatgctccagcttttgaaactgcaggcacatatcttagactggttaaaattaatgattggaccgaaataacccagttcattttggaacatcgagctagggcaagctgtaaatatgccctgcctctccggattccacctgccgcttgtttgactagcaaagcttatcagcaaggagtaaccgtagattctattggtatgctgcctaggttcattccagagaatcaacggactgttgctctctattccctgaagattgcgggatggcatgggcctaaaccaccctatacatcaactcttttaccgcccgaactctctgatacgacaaatgctacacagccagagctggtgcccgaggatccagaagatagtgctctgctggaagacccggctggcacagtctccagccaaattccacccaattggcatattccctcaattcaagatgtggcccctcatcatgctcctgctgcaccgagtaatccaggttgagagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 1474 nt, synthetic construct
  misc_feature 1..1474: note = Codon-optimized ICP4 of HSV-2 RNA sequence including UTRs and polyA, all uridine residues are 1-methyl-pseudouridine (m1f)
  source 1..1474: mol_type = other RNA
taatacgactcactataaggactcactatttgttttcgcgcccagttgcaaaaagtgtcgtcaccatggcagttcgcagagctggccggcaaccacctcgccctcttggcggtgccagaggcggcggcggtgccggtcctgtaagaggtctgggtcggcctggagccagagtaggggctagagcaggccgcagaggagcggctgtaagacccgaccatgcagcggcagtgcatgctgggagaggtggggatggagttgcaccagaaccagctcgcggggccagaggacgcggtgcaggtcctggtctcttgcctgacttaggtcgcggggcccagcaacaattactgcatttacgccagcgcggagccggaagggccccacctggagtgagacacggtggtgggcctctccgattgggacccggcgccaggggtggacgaagaggacatgaaccccctttgcgaccacgtgcggaagggttgcccgccgatcaacctgcaccaaggctgagagctcctgctggggcccgcgagagaggagctgatagaggtgcgaatccaaggagaagacggcgccgccaaccacccagaagaaggcggcggcctcgggaagctcgacgtagaagaaggcctgttgcagtgggggggggagttgccggtcgcagagctcgaggggcacgccgactgagacgtaggggcggagccagacgtcccggcgcacctgaacgaagggctcgattgagagctggtcggggacggcgaaggcgacgccgtagacgccgtaggcgacgatggtggcgcagacctgcccgcggaggaggccctagagggagaggcgttccgggccgtttgccgcgggatccaggtggggcaggaggagggcttaggcgcaggcctggcggacgggcaggggcagggcggtcaccagcgaggagagcacctgcccctggcgccagaggcagagggcggccagcagcaaggcgaagggccccgcctgccaggttagccgcaagggctgcggtgagagcccgaagggccggcgccgatgcaccagcccgcggccccgcaagaggccgaaggcaaaggggccgacgaggaaggcgcgcccgtagagaaccgggcaggcggggccctggtccgggcgctgcagcagaaccagcaccagcagaacttcgacggagaagaaggagagggcctgcgctccctgagccagaacccgcctaggagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - protein, 1320 aa, Human alphaherpesvirus 2
MSAEQRKKKTTTTTQGRGAEVTMADEDGGRLRAAAETTGGPGSPDPADGPPPTPNPDRRPAARPGFGWHGGPEENEDEDDDAAADADEAAPASGEAVDEPAADGVVSPRQLALLASMVDEAVRTIPSPPPERDGAEEEAARSPSPPRTPSMRADYGEENDDDDDDDDDDRDAGRWVRGPETTSAVRGAYPDPMASLSPRPPAPRRHHHRHHRRRRAPRRRSAASDSSKSGSSSSASSASSSASSSSSASASSSDDDDDDDAAARAPASAADHAAGGTLGADDEEAGVPARAPGAAPRPSPPRAEPAPGAGAGSARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPAAAAAPLPSAAASPADERAVPAGYGAAGVLAALGRLSAAPASAPAGADDDDDDDGPGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGTAAGLATPPRREPVDMDAELEDDDDGLFGE

SEQ ID NO: 32 - RNA, 291 nt, synthetic construct
  source 1..291: mol_type = other RNA
atgggtttggcttttagtggagctaggccttgttgttgtaggcataatgtgattattactgatggtggcgaagtggtgtcactcactgctcatgagttcgatgttgttgatatagaaagtgaggaagaagggaatttctatgtcccgccagatatgcgggtagtgactagagcccccggcccacaatatagaagagccagcgatccaccctctcgacatactcgacgacgagatccagatgtagcacggcccccagcgaccttgaccccacccctgtccgattccgagtga

SEQ ID NO: 33 - RNA, 1119 nt, synthetic construct
  source 1..1119: mol_type = other RNA
atggctcaaagagccctgtggagaccacaagccacccctggaccacccggcgctgccgctccacctggacatagaggtgctcccccagatgctagagctcctgatccaggtccggaagcagatttggttgccagaattgcgaatagcgtgtttgtctggagagttgtgcgcggtgatgaaaggctgaaaatcttccgctgtctgactgtacttacggaacccttatgtcaagtcgcattacccgatcctgatcctgaaagagctctgttttgtgaaatctttctgtatctcacccgcccgaaagctctccggctcccgtctaataccttcttcgcgatcttcttcttcaatagagaaaggcggtattgtgcaacagtgcatcttcggtctgttacccatccgagaacaccactcctctgtacactggcttttggacatctcgaggcggcttctccacccgaagagactcctgatccagcagctgagcaactggcagatgaaccagttgcacatgaacttgatggggcatatctcgttccaactgagccaccaccaaacccaggtgcctgttgcgcgttgggaccaggagcttggtggcatctgccaggtgggcggatctattgttgggctatggacgacgatctcggtagtctgtgcccacccgggagtcgtgctagacacctcggctggcttctttcacgcatcacggatccacccggggggggcggggcatgtgctcccacagctcatattgacagtgctaatgccctttggcgagctccagcagttgctgaagcttgcccgtgtgtggctccttgtatgtggagcaacatggcgcaacggacacttgctgtgcgcggtgatgcctcactctgccaattgctgtttggtcatcctgtcgatgctgtgatacttcgtcaagccactagacgtcccaggattacagcacatctccatgaagtggtggtaggaagggatggagcagagagtgtgattaggcccacgtccgctggctggagactgtgtgtgttaagctcctacacaagtaggcttttcgctactagttgtccggccgttgcaagagctgtcgcaagggcatccagttctgactataagtaa

SEQ ID NO: 34 - RNA, 1599 nt, synthetic construct
  source 1..1599: mol_type = other RNA
atggaactgtcctacgcaacaactcttcatcatagagatgtagtcttctatgtgaccgccgatcggaatagggcatatttcgtctgtggcggtagtgtctactctgtgggcagaccgagggacagccaacctggcgagatcgctaaattcgggctcgtcgtgaggggtaccggaccaaaggatagaatggtggcaaattatgtgcggtccgaattgaggcaaagaggtctccgcgatgttagacctgttggcgaagatgaagtatttctcgattccgtttgcttgttgaatccaaatgtttcctcagaacgcgatgtaatcaacactaatgatgtcgaggtcttggatgagtgtctcgctgagtattgtacaagcctcaggacgagtcccggtgttttagtcacaggagttagggtcagagcacgcgatagagtgattgaattattcgaacatcccgctattgtaaatatctctagccgatttgcatatacgccttctccatatgtgtttgctctcgctcaagcccatctgccacgtctgccatcctcactcgaaccgctcgtatctgggttgttcgatgggataccggcaccaagacaaccgctcgatgctcgagatcgccgtaccgatgtggttataaccgggacaagagcaccacggcctatggcgggaacaggcgcaggcggagccggcgctaaacgcgctacagtgtccgaatttgtacaggtcaaacatatagatagagtggtctcacctagtgtaagcagtgcgccaccccctagtgcgcctgatgcttcacttcccccgcccggactgcaagaagcggcaccaccaggaccacccctgagagaattatggtgggtcttttatgctggagatagggctcttgaagaacctcatgcagaaagcggcctgaccagggaagaagtgagggctgtccacggttttagagaacaagcctggaaattattcggctctgttggagcccccagggccttcctgggcgcagcattagcactctctcccacacagaaactggcagtgtattattacttgatacatcgcgaaaggagaatgtctccgtttccggcccttgtacgcttggtgggaagatatattcaaaggcatggactctatgtgcctgccccagatgagcccaccctcgctgacgctatgaatggtctgtttcgggatgctcttgctgcaggcacagttgcagaacaactgttaatgtttgatttgttaccccctaaagatgtaccagttggatctgatgcaagagctgatagtgctgcactcctcagattcgtcgatagccagcggctcacacccggtggcagcgtgagcccagaacatgtgatgtatctcggggcatttcttggtgttctttatgctgggcatggcagacttgctgccgcgactcacactgctcggttgacaggtgtaacaagcctcgttttgacagttggcgatgtagataggatgtcagccttcgatcgaggtccagccggcgcagcaggacgaactagaactgcaggatatctcgatgctctcttaacagtgtgtcttgcccgggctcaacatgggcaatccgtttag

SEQ ID NO: 35 - protein, 10 aa, Synthetic construct
FYVPPDMRVV

SEQ ID NO: 36 - protein, 15 aa, Synthetic construct
CEIFLYLTRPKALRL

SEQ ID NO: 37 - protein, 15 aa, Synthetic construct
FFAIFFFNRERRYCA

SEQ ID NO: 38 - protein, 9 aa, Synthetic construct
VTHPRTPLL

SEQ ID NO: 39 - protein, 9 aa, Synthetic construct
LGPGAWWHL

SEQ ID NO: 40 - protein, 9 aa, Synthetic construct
RRPRITAHL

SEQ ID NO: 41 - protein, 15 aa, Synthetic construct
RDVVFYVTADRNRAY

SEQ ID NO: 42 - protein, 9 aa, Synthetic construct
SQPGEIAKF

SEQ ID NO: 43 - protein, 9 aa, Synthetic construct
AYTPSPYVF

SEQ ID NO: 44 - protein, 12 aa, Synthetic construct
HLPRLPSSLEPL

SEQ ID NO: 45 - protein, 9 aa, Synthetic construct
PGPPLRELW

SEQ ID NO: 46 - protein, 15 aa, Synthetic construct
ELWWVFYAGDRALEE

SEQ ID NO: 47 - protein, 15 aa, Synthetic construct
AVYYYLIHRERRMSP

SEQ ID NO: 48 - protein, 15 aa, Synthetic construct
HRERRMSPFPALVRL

SEQ ID NO: 49 - protein, 9 aa, Synthetic construct
VSPEHVMYL

SEQ ID NO: 50 - protein, 17 aa, Synthetic construct
AAATHTARLTGVTSLVL

SEQ ID NO: 51 - protein, 12 aa, Synthetic construct
RTAGYLDALLTV

SEQ ID NO: 52 - DNA, 22 bp, synthetic construct
  source 1..22: mol_type = other DNA
tcgtcgttgtcgttttgtcgtt

SEQ ID NO: 53 - DNA, 23 bp, synthetic construct
  source 1..23: mol_type = other DNA
ggcagaagcctactactcggaaa

SEQ ID NO: 54 - DNA, 18 bp, synthetic construct
  source 1..18: mol_type = other DNA
ccatgcgcacgaggaagt

SEQ ID NO: 55 - DNA, 13 bp, synthetic construct
  source 1..13: mol_type = other DNA
  modified_base 1: mod_base = OTHER; note = 6-carboxyfluorescein; note = nucleotide covalently linked to the the methionine in SEQ ID NO:65, position 13
  modified_base 13: mod_base = OTHER; note = nucleotide is covalently linked to the methionine in position 1 in SEQ ID NO:85
cgaggccgccaac

SEQ ID NO: 56 - DNA, 68 bp, synthetic construct
  source 1..68: mol_type = other DNA
taatacgactcactataaggactcactatttgttttcgcgcccagttgcaaaaagtgtcgtcaccatg

SEQ ID NO: 57 - DNA, 105 bp, synthetic construct
  source 1..105: mol_type = other DNA
ttttttttttttttttttttttttttttttttttttttttttttttttttttttttttttttttttttttttttttttttttttttgtaatccagaggttgattg

SEQ ID NO: 58 - RNA, 90 nt, synthetic construct
  source 1..90: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - RNA, 65 nt, synthetic construct
  source 1..65: mol_type = other RNA
taatacgactcactataaggactcactatttgttttcgcgcccagttgcaaaaagtgtcgtcacc

SEQ ID NO: 60 - RNA, 161 nt, synthetic construct
  source 1..161: mol_type = other RNA
gagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattac

SEQ ID NO: 61 - RNA, 607 nt, synthetic construct; identical to SEQ ID NO: 26
  source 1..607: mol_type = other RNA

SEQ ID NO: 62 - RNA, 1435 nt, synthetic construct; identical to SEQ ID NO: 27
  source 1..1435: mol_type = other RNA

SEQ ID NO: 63 - RNA, 1915 nt, synthetic construct; identical to SEQ ID NO: 28
  source 1..1915: mol_type = other RNA

SEQ ID NO: 64 - RNA, 1885 nt, synthetic construct
  source 1..1885: mol_type = other RNA
taatacgactcactataaggactcactatttgttttcgcgcccagttgcaaaaagtgtcgtcaccatggccagaggcgccggcctggtgttcttcgtgggcgtgtgggttgtgtcttgcctggccgccgccccccggaccagctggaagcgggtgaccagcggagaggacgtggtgctgctgcctgcccccgctgggcctgaggaaagaacccgcgcccacaagctcctgtgggccgctgaacctctggacgcttgcggccccctcagaccctcttgggtggccctgtggcctcctagacgggtgctggaaaccgtggttgacgctgcctgcatgagagcccccgagcctctggccatcgcttattctccacctttccccgccggcgacgaaggcctgtacagcgagctggcctggcgggacagagtggccgtcgtgaatgagtccctggtcatctacggcgctctggaaaccgactccggactgtatacactgagcgtggtcggcctgagcgacgaggccagacaagtggcttctgtggtgctggtggtggaacccgctccagtgcctacacctacccccgacgactacgacgaggaagatgacgccggcgtgagcgagagaacccctgtgagcgtcccacctccgactccacctagacgcccccctgtggcccctcctacacaccccagagtgatccctgaggtgtcccacgtgcggggcgtgaccgtgcacatggaaacacccgaggctatcctgtttgcccctggcgagacattcggcaccaacgtgagcatccacgccattgcccatgatgatggcccttacgccatggacgtggtgtggatgagattcgacgtgcctagcagctgtgccgagatgagaatctacgaggcctgtctgtaccacccacagctgcctgagtgcctgtcccctgccgatgccccttgcgccgttagctcttgggcctacagactggctgtccggagctacgccggatgtagcagaaccacacctcctccaagatgcttcgccgaggcgagaatggaaccagtgcctggcctggcctggctggccagcaccgtgaacctggaattccagcacgccagcccccagcacgccggactgtacctgtgcgtggtctacgtggacgatcacatccacgcttggggccacatgacaatcagcaccgccgcccagtacagaaacgccgtggtggaacagcatctgcctcaaagacagcctgaacccgttgagcctaccagacctcacgtgcgggcccctcctcctgccccaagcgccaggggccccctgagaagacggcggagctggcgggccgtgaaaagcagagcctccgccacaggccctacctacatcagagtggccgacagcgaactgtacgctgattggtcttctgatagcgagggagaacgcgacggcagcctgtggcaggacccccctgagcggcctgatagccctagcaccaacggcagcggcttcgagatcctgtcgcctacagccccctccgtgtacccccactctgagggcagaaagagccggagacctctgaccacctttggttctggatctcccggcagaagacacagccaggccagctacagcagcgtgctgtgggagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - RNA, 2506 nt, synthetic construct
  source 1..2506: mol_type = other RNA
taatacgactcactataaggactcactatttgttttcgcgcccagttgcaaaaagtgtcgtcaccatgcggggcggcggcctgatctgcgccctggtggtgggcgccctggtcgctgccgtggccagcgccgcccctgccgcccctgccgctcctagagccagcggcggagtggctgccaccgtggccgccaatggcggccctgctagcagacctcctcctgtgcccagccctgccactaccaaggcccggaagagaaagaccaagaagcctccaaagcggccagaagccacacctccaccagatgccaacgccacagtggccgctggccacgccacactgcgggcccacctgagagagatcaaggtggaaaacgccgacgcccagttctacgtgtgccccccacctaccggcgccaccgtggtacaatttgagcagcctagaaggtgtcctaccagacctgagggccagaactacaccgagggcatcgccgtggtctttaaggaaaacatcgccccctacaagttcaaggccaccatgtactacaaggacgtgacagttagccaagtgtggttcggccatagatacagccagttcatgggcatcttcgaggacagagcacccgtgcctttcgaggaagtgattgataagatcaacgccaagggcgtgtgtagaagcacagccaagtatgtgcggaacaacatggaaaccaccgccttccaccgggacgaccacgagacagacatggaacttaaacctgctaaagtcgccacccgcaccagcagaggctggcacacaaccgatctgaagtataatccttctagagtggaagcttttcacagatacggcaccaccgttaactgcatcgtggaagaagtggacgcccgcagcgtgtatccttacgacgagttcgtgctggccaccggcgacttcgtgtacatgagccccttctacggctacagagaaggcagccacaccgagcacacgagctacgccgccgaccggttcaagcaggtcgatggattttacgcgcgtgatctgacaaccaaggctcgggccacatcccctacaacaagaaacctgctgacgacccctaaattcacagtggcctgggactgggtccccaaacggcccgccgtgtgcaccatgacaaagtggcaggaggtagatgagatgctgcgggccgagtacggcgggagcttcagattcagcagcgacgccatcagcaccacattcaccaccaacctgacacagtattctctgagccgggtggacctgggcgactgcatcggaagagacgccagagaggctatcgacagaatgttcgccagaaagtacaacgccacccacatcaaagtgggacaacctcagtactacctggccacaggcggattcctgatcgcctaccagcctctgctgtccaacaccctggctgaactgtacgtgcgggaatacatgcgagaacaggacagaaagcccagaaatgctacccctgcccctctgcgggaagcccctagcgccaacgcctctgtggagagaatcaaaaccacctctagcatcgagttcgcccggctgcagtttacctacaaccacatccagcggcacgtgaacgacatgctgggcagaatcgccgtggcctggtgcgagctgcaaaatcatgagctgaccctgtggaatgaggccagaaagctgaaccccaacgccatcgccagcgccaccgtgggccggagagtgagcgcgagaatgctgggcgatgtgatggccgtgtccacctgtgtgcccgtggctcctgacaacgtgatcgtgcagaacagcatgcgggtgtcttctagacctggaacctgctactccaggcccctggtgtcttttcggtacgaggatcagggccccctgatcgagggccagctgggcgagaacaatgagctgagactgaccagagatgctctggaaccctgcacagtgggccaccgaagatacttcatcttcggcggaggatacgtgtacttcgaggaatatgcttacagccaccagctgtccagagccgacgtgaccactgtgtctaccttcatcgacctcaacattacaatgctggaagatcacgaattcgtgccactgggagtttacaccagacacgagatcaaagacagcggcctgctggactacaccgaggtgcagagacggaaccagttgcacgacctgagatttgccgacatcgatacagtgatcagagccgatgctaatgccgctatgttcgctgagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - RNA, 1339 nt, synthetic construct; identical to SEQ ID NO: 29
  source 1..1339: mol_type = other RNA

SEQ ID NO: 67 - RNA, 1569 nt, synthetic construct
  source 1..1569: mol_type = other RNA
atggccagaggcgccggcctggtgttcttcgtgggcgtgtgggttgtgtcttgcctggccgccgccccccggaccagctggaagcgggtgaccagcggagaggacgtggtgctgctgcctgcccccgctgggcctgaggaaagaacccgcgcccacaagctcctgtgggccgctgaacctctggacgcttgcggccccctcagaccctcttgggtggccctgtggcctcctagacgggtgctggaaaccgtggttgacgctgcctgcatgagagcccccgagcctctggccatcgcttattctccacctttccccgccggcgacgaaggcctgtacagcgagctggcctggcgggacagagtggccgtcgtgaatgagtccctggtcatctacggcgctctggaaaccgactccggactgtatacactgagcgtggtcggcctgagcgacgaggccagacaagtggcttctgtggtgctggtggtggaacccgctccagtgcctacacctacccccgacgactacgacgaggaagatgacgccggcgtgagcgagagaacccctgtgagcgtcccacctccgactccacctagacgcccccctgtggcccctcctacacaccccagagtgatccctgaggtgtcccacgtgcggggcgtgaccgtgcacatggaaacacccgaggctatcctgtttgcccctggcgagacattcggcaccaacgtgagcatccacgccattgcccatgatgatggcccttacgccatggacgtggtgtggatgagattcgacgtgcctagcagctgtgccgagatgagaatctacgaggcctgtctgtaccacccacagctgcctgagtgcctgtcccctgccgatgccccttgcgccgttagctcttgggcctacagactggctgtccggagctacgccggatgtagcagaaccacacctcctccaagatgcttcgccgaggcgagaatggaaccagtgcctggcctggcctggctggccagcaccgtgaacctggaattccagcacgccagcccccagcacgccggactgtacctgtgcgtggtctacgtggacgatcacatccacgcttggggccacatgacaatcagcaccgccgcccagtacagaaacgccgtggtggaacagcatctgcctcaaagacagcctgaacccgttgagcctaccagacctcacgtgcgggcccctcctcctgccccaagcgccaggggccccctgagaagacggcggagctggcgggccgtgaaaagcagagcctccgccacaggccctacctacatcagagtggccgacagcgaactgtacgctgattggtcttctgatagcgagggagaacgcgacggcagcctgtggcaggacccccctgagcggcctgatagccctagcaccaacggcagcggcttcgagatcctgtcgcctacagccccctccgtgtacccccactctgagggcagaaagagccggagacctctgaccacctttggttctggatctcccggcagaagacacagccaggccagctacagcagcgtgctgtgg

SEQ ID NO: 68 - RNA, 2190 nt, synthetic construct
  source 1..2190: mol_type = other RNA
atgcggggcggcggcctgatctgcgccctggtggtgggcgccctggtcgctgccgtggccagcgccgcccctgccgcccctgccgctcctagagccagcggcggagtggctgccaccgtggccgccaatggcggccctgctagcagacctcctcctgtgcccagccctgccactaccaaggcccggaagagaaagaccaagaagcctccaaagcggccagaagccacacctccaccagatgccaacgccacagtggccgctggccacgccacactgcgggcccacctgagagagatcaaggtggaaaacgccgacgcccagttctacgtgtgccccccacctaccggcgccaccgtggtacaatttgagcagcctagaaggtgtcctaccagacctgagggccagaactacaccgagggcatcgccgtggtctttaaggaaaacatcgccccctacaagttcaaggccaccatgtactacaaggacgtgacagttagccaagtgtggttcggccatagatacagccagttcatgggcatcttcgaggacagagcacccgtgcctttcgaggaagtgattgataagatcaacgccaagggcgtgtgtagaagcacagccaagtatgtgcggaacaacatggaaaccaccgccttccaccgggacgaccacgagacagacatggaacttaaacctgctaaagtcgccacccgcaccagcagaggctggcacacaaccgatctgaagtataatccttctagagtggaagcttttcacagatacggcaccaccgttaactgcatcgtggaagaagtggacgcccgcagcgtgtatccttacgacgagttcgtgctggccaccggcgacttcgtgtacatgagccccttctacggctacagagaaggcagccacaccgagcacacgagctacgccgccgaccggttcaagcaggtcgatggattttacgcgcgtgatctgacaaccaaggctcgggccacatcccctacaacaagaaacctgctgacgacccctaaattcacagtggcctgggactgggtccccaaacggcccgccgtgtgcaccatgacaaagtggcaggaggtagatgagatgctgcgggccgagtacggcgggagcttcagattcagcagcgacgccatcagcaccacattcaccaccaacctgacacagtattctctgagccgggtggacctgggcgactgcatcggaagagacgccagagaggctatcgacagaatgttcgccagaaagtacaacgccacccacatcaaagtgggacaacctcagtactacctggccacaggcggattcctgatcgcctaccagcctctgctgtccaacaccctggctgaactgtacgtgcgggaatacatgcgagaacaggacagaaagcccagaaatgctacccctgcccctctgcgggaagcccctagcgccaacgcctctgtggagagaatcaaaaccacctctagcatcgagttcgcccggctgcagtttacctacaaccacatccagcggcacgtgaacgacatgctgggcagaatcgccgtggcctggtgcgagctgcaaaatcatgagctgaccctgtggaatgaggccagaaagctgaaccccaacgccatcgccagcgccaccgtgggccggagagtgagcgcgagaatgctgggcgatgtgatggccgtgtccacctgtgtgcccgtggctcctgacaacgtgatcgtgcagaacagcatgcgggtgtcttctagacctggaacctgctactccaggcccctggtgtcttttcggtacgaggatcagggccccctgatcgagggccagctgggcgagaacaatgagctgagactgaccagagatgctctggaaccctgcacagtgggccaccgaagatacttcatcttcggcggaggatacgtgtacttcgaggaatatgcttacagccaccagctgtccagagccgacgtgaccactgtgtctaccttcatcgacctcaacattacaatgctggaagatcacgaattcgtgccactgggagtttacaccagacacgagatcaaagacagcggcctgctggactacaccgaggtgcagagacggaaccagttgcacgacctgagatttgccgacatcgatacagtgatcagagccgatgctaatgccgctatgttcgct

SEQ ID NO: 69 - RNA, 1021 nt, synthetic construct
  source 1..1021: mol_type = other RNA
atgggccgcctcactagtggggttggtactgctgcgttactggtcgtggctgtcgggcttagagtggtttgtgctaagtatgcacttgctgatccatcactgaaaatggcagacccaaaccgcttcaggggtaagaatttgcccgtgctggatcaattgacagatccaccgggcgttaaaagagtctatcatatccaacccagtctcgaagatccttttcaacccccttccattcccattaccgtatattatgccgtattagagcgggcttgtcgttccgtacttttgcacgcgccatccgaagcacctcaaattgtcagaggagcatccgatgaagctagaaaacatacctataatctcacaatagcttggtacagaatgggcgataactgtgccattccaattacagtcatggagtatacggaatgtccttataataaaagtctcggagtatgtccaattagaacccaaccacggtggtcatattacgattccttcagtgcggtttctgaagacaatctcgggtttctcatgcatgctccagcttttgaaactgcaggcacatatcttagactggttaaaattaatgattggaccgaaataacccagttcattttggaacatcgagctagggcaagctgtaaatatgccctgcctctccggattccacctgccgcttgtttgactagcaaagcttatcagcaaggagtaaccgtagattctattggtatgctgcctaggttcattccagagaatcaacggactgttgctctctattccctgaagattgcgggatggcatgggcctaaaccaccctatacatcaactcttttaccgcccgaactctctgatacgacaaatgctacacagccagagctggtgcccgaggatccagaagatagtgctctgctggaagacccggctggcacagtctccagccaaattccacccaattggcatattccctcaattcaagatgtggcccctcatcatgctcctgctgcaccgagtaatccaggtt

SEQ ID NO: 70 - DNA, 294 bp, Human alphaherpesvirus 2
  source 1..294: mol_type = other DNA
atgggtttggcttttagtggagctaggccttgttgttgtaggcataatgtgattattactgatggtggcgaagtggtgtcactcactgctcatgagttcgatgttgttgatatagaaagtgaggaagaagggaatttctatgtcccgccagatatgcgggtagtgactagagcccccggcccacaatatagaagagccagcgatccaccctctcgacatactcgacgacgagatccagatgtagcacggcccccagcgaccttgaccccacccctgtccgattccgagtgataa

SEQ ID NO: 71 - DNA, 1122 bp, Human alphaherpesvirus 2
  source 1..1122: mol_type = other DNA
atggctcaaagagccctgtggagaccacaagccacccctggaccacccggcgctgccgctccacctggacatagaggtgctcccccagatgctagagctcctgatccaggtccggaagcagatttggttgccagaattgcgaatagcgtgtttgtctggagagttgtgcgcggtgatgaaaggctgaaaatcttccgctgtctgactgtacttacggaacccttatgtcaagtcgcattacccgatcctgatcctgaaagagctctgttttgtgaaatctttctgtatctcacccgcccgaaagctctccggctcccgtctaataccttcttcgcgatcttcttcttcaatagagaaaggcggtattgtgcaacagtgcatcttcggtctgttacccatccgagaacaccactcctctgtacactggcttttggacatctcgaggcggcttctccacccgaagagactcctgatccagcagctgagcaactggcagatgaaccagttgcacatgaacttgatggggcatatctcgttccaactgagccaccaccaaacccaggtgcctgttgcgcgttgggaccaggagcttggtggcatctgccaggtgggcggatctattgttgggctatggacgacgatctcggtagtctgtgcccacccgggagtcgtgctagacacctcggctggcttctttcacgcatcacggatccacccggggggggcggggcatgtgctcccacagctcatattgacagtgctaatgccctttggcgagctccagcagttgctgaagcttgcccgtgtgtggctccttgtatgtggagcaacatggcgcaacggacacttgctgtgcgcggtgatgcctcactctgccaattgctgtttggtcatcctgtcgatgctgtgatacttcgtcaagccactagacgtcccaggattacagcacatctccatgaagtggtggtaggaagggatggagcagagagtgtgattaggcccacgtccgctggctggagactgtgtgtgttaagctcctacacaagtaggcttttcgctactagttgtccggccgttgcaagagctgtcgcaagggcatccagttctgactataagtagtaa

SEQ ID NO: 72 - DNA, 1602 bp, Human alphaherpesvirus 2
  source 1..1602: mol_type = other DNA
atggaactgtcctacgcaacaactcttcatcatagagatgtagtcttctatgtgaccgccgatcggaatagggcatatttcgtctgtggcggtagtgtctactctgtgggcagaccgagggacagccaacctggcgagatcgctaaattcgggctcgtcgtgaggggtaccggaccaaaggatagaatggtggcaaattatgtgcggtccgaattgaggcaaagaggtctccgcgatgttagacctgttggcgaagatgaagtatttctcgattccgtttgcttgttgaatccaaatgtttcctcagaacgcgatgtaatcaacactaatgatgtcgaggtcttggatgagtgtctcgctgagtattgtacaagcctcaggacgagtcccggtgttttagtcacaggagttagggtcagagcacgcgatagagtgattgaattattcgaacatcccgctattgtaaatatctctagccgatttgcatatacgccttctccatatgtgtttgctctcgctcaagcccatctgccacgtctgccatcctcactcgaaccgctcgtatctgggttgttcgatgggataccggcaccaagacaaccgctcgatgctcgagatcgccgtaccgatgtggttataaccgggacaagagcaccacggcctatggcgggaacaggcgcaggcggagccggcgctaaacgcgctacagtgtccgaatttgtacaggtcaaacatatagatagagtggtctcacctagtgtaagcagtgcgccaccccctagtgcgcctgatgcttcacttcccccgcccggactgcaagaagcggcaccaccaggaccacccctgagagaattatggtgggtcttttatgctggagatagggctcttgaagaacctcatgcagaaagcggcctgaccagggaagaagtgagggctgtccacggttttagagaacaagcctggaaattattcggctctgttggagcccccagggccttcctgggcgcagcattagcactctctcccacacagaaactggcagtgtattattacttgatacatcgcgaaaggagaatgtctccgtttccggcccttgtacgcttggtgggaagatatattcaaaggcatggactctatgtgcctgccccagatgagcccaccctcgctgacgctatgaatggtctgtttcgggatgctcttgctgcaggcacagttgcagaacaactgttaatgtttgatttgttaccccctaaagatgtaccagttggatctgatgcaagagctgatagtgctgcactcctcagattcgtcgatagccagcggctcacacccggtggcagcgtgagcccagaacatgtgatgtatctcggggcatttcttggtgttctttatgctgggcatggcagacttgctgccgcgactcacactgctcggttgacaggtgtaacaagcctcgttttgacagttggcgatgtagataggatgtcagccttcgatcgaggtccagccggcgcagcaggacgaactagaactgcaggatatctcgatgctctcttaacagtgtgtcttgcccgggctcaacatgggcaatccgtttagtaa

SEQ ID NO: 73 - DNA, 1251 bp, synthetic construct
  source 1..1251: mol_type = other DNA
atggccaggggcgccggcctggtgttcttcgtgggcgtgtgggtggtgagctgcctggccgccgcccccaggaccagctggaagagggtgaccagcggcgaggacgtggtgctgctgcccgcccccgccggccccgaggagaggaccagggcccacaagctgctgtgggccgccgagcccctggacgcctgcggccccctgaggcccagctgggtggccctgtggccccccaggagggtgctggagaccgtggtggacgccgcctgcatgagggcccccgagcccctggccatcgcctacagcccccccttccccgccggcgacgagggcctgtacagcgagctggcctggagggacagggtggccgtggtgaacgagagcctggtgatctacggcgccctggagaccgacagcggcctgtacaccctgagcgtggtgggcctgagcgacgaggccaggcaggtggccagcgtggtgctggtggtggagcccgcccccgtgcccacccccacccccgacgactacgacgaggaggacgacgccggcgtgagcgagaggacccccgtgagcgtgcccccccccaccccccccaggaggccccccgtggccccccccacccaccccagggtgatccccgaggtgagccacgtgaggggcgtgaccgtgcacatggagacccccgaggccatcctgttcgcccccggcgagaccttcggcaccaacgtgagcatccacgccatcgcccacgacgacggcccctacgccatggacgtggtgtggatgaggttcgacgtgcccagcagctgcgccgagatgaggatctacgaggcctgcctgtaccacccccagctgcccgagtgcctgagccccgccgacgccccctgcgccgtgagcagctgggcctacaggctggccgtgaggagctacgccggctgcagcaggaccacccccccccccaggtgcttcgccgaggccaggatggagcccgtgcccggcctggcctggctggccagcaccgtgaacctggagttccagcacgccagcccccagcacgccggcctgtacctgtgcgtggtgtacgtggacgaccacatccacgcctggggccacatgaccatcagcaccgccgcccagtacaggaacgccgtggtggagcagcacctgccccagaggcagcccgagcccgtggagcccaccaggccccacgtgagggcccccccccccgcccccagcgccaggggccccctgagg

SEQ ID NO: 74 - DNA, 2192 bp, Human alphaherpesvirus 2
  source 1..2192: mol_type = other DNA
atgcgcggtggcggattgatctgcgccctggtggtgggtgcactggtggctgctgtcgcttcagctgctcctgctgctcctgcagctcctcgcgctagtggtggcgttgctgccaccgtggcagcgaacggaggtccagctagccgtcctcccccagtcccgtctcctgcaaccactaaggcacgcaagcgtaaaaccaagaaaccgcctaagaggcctgaagcaactcccccaccggacgcaaacgctactgtggctgctggtcacgctactctcagggctcatttgagagagatcaaggttgaaaacgctgacgcccagttctacgtgtgccctcccccaacaggtgcaactgttgtgcagttcgaacagcctcgcaggtgtcctaccaggcctgagggtcaaaactacactgaaggcatcgctgtcgttttcaaggagaacattgccccatacaagttcaaagcaaccatgtactacaaagacgtgactgtctcgcaagtgtggttcggacaccgttactcccaattcatgggtatcttcgaggacagggctcccgttccattcgaggaagtgatcgataagattaacgccaaaggcgtgtgcaggtccacagcaaagtacgtcagaaacaacatggagacaacggctttccaccgcgacgatcatgagaccgacatggaattgaagcctgcgaaagtggctacaaggacgtcgagaggctggcacaccactgatctgaagtacaacccctccagggttgaagccttccatagatacggaacaacggtgaactgtatcgttgaggaagtggatgcccgctcagtgtacccctacgacgagttcgtcctggctaccggagatttcgtttacatgagtccattctacggatacagggagggttcgcacaccgaacatacttcctacgcagcggacagattcaagcaggtggatggtttctacgctcgcgacctgaccactaaagcacgtgcgacttctcctacaacgaggaacctgctcaccactcccaagttcacagtcgcttgggactgggttccaaagaggccggccgtctgcaccatgactaaatggcaggaggttgacgaaatgctgagagctgaatacggcggaagcttccgcttctccagcgatgccatctctacaacgttcaccactaacctgactcaatactccctcagccgtgtggacctgggtgactgtatcggacgcgatgcccgtgaggcaattgaccgcatgttcgcgcgtaagtacaacgctacacacatcaaagtgggccagccacaatactacctcgcgacgggtggcttcttgattgcttaccagcctttgctgtcaaacactctcgctgaattgtacgtcagggagtacatgagagaacaagacaggaagccaagaaacgcgacaccggctcctctgagagaggctccgtctgctaacgcctcagtggaacgcatcaagacaacctcctccattgagttcgctcgtctccagttcacctacaaccacatccaacgtcatgtgaacgacatgctgggaaggattgcagtcgcgtggtgcgagctccagaaccacgaactgaccctctggaacgaggccaggaagctcaacccaaacgctatcgcctcagcaactgtcggcaggagagttagtgcgagaatgttgggagacgtcatggctgttagcacctgcgttcctgtggcccccgataacgtcattgttcaaaactctatgcgcgtcagttcgcgtccaggaacttgttactcccgcccgctcgttagcttccgttacgaggaccagggtccactgatcgaaggtcaactcggcgagaacaacgaattgcgcctgacacgtgatgctctggagccttgtacggtcggtcaccgccgttacttcattttcggaggtggctacgtgtacttcgaggaatacgcatacagtcaccagttgtcgcgcgctgacgtgaccactgtctccacattcatcgacttgaacattacgatgctggaggatcacgaattcgtgcccctgggtgtctacacacgtcatgaaatcaaggacagcggcctcttggattacaccgaggtccagaggagaaaccaactgcacgacctcagattcgctgacatcgatactgtgattcgcgcggatgctaacgctgccatgttcgctgg

SEQ ID NO: 75 - DNA, 1182 bp, Human alphaherpesvirus 2
  source 1..1182: mol_type = other DNA
atggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgttaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggcgctggtcatcggcggtattgcgttttgggtacgccgccgcgctcagatggcccccaagcgcctacgtctcccccacatccgggatgacgacgcgcccccctcgcaccagccattgttttactag

SEQ ID NO: 76 - DNA, 1572 bp, Human alphaherpesvirus 2
  source 1..1572: mol_type = other DNA
atggccaggggcgccggcctggtgttcttcgtgggcgtgtgggtggtgagctgcctggccgccgcccccaggaccagctggaagagggtgaccagcggcgaggacgtggtgctgctgcccgcccccgccggccccgaggagaggaccagggcccacaagctgctgtgggccgccgagcccctggacgcctgcggccccctgaggcccagctgggtggccctgtggccccccaggagggtgctggagaccgtggtggacgccgcctgcatgagggcccccgagcccctggccatcgcctacagcccccccttccccgccggcgacgagggcctgtacagcgagctggcctggagggacagggtggccgtggtgaacgagagcctggtgatctacggcgccctggagaccgacagcggcctgtacaccctgagcgtggtgggcctgagcgacgaggccaggcaggtggccagcgtggtgctggtggtggagcccgcccccgtgcccacccccacccccgacgactacgacgaggaggacgacgccggcgtgagcgagaggacccccgtgagcgtgcccccccccaccccccccaggaggccccccgtggccccccccacccaccccagggtgatccccgaggtgagccacgtgaggggcgtgaccgtgcacatggagacccccgaggccatcctgttcgcccccggcgagaccttcggcaccaacgtgagcatccacgccatcgcccacgacgacggcccctacgccatggacgtggtgtggatgaggttcgacgtgcccagcagctgcgccgagatgaggatctacgaggcctgcctgtaccacccccagctgcccgagtgcctgagccccgccgacgccccctgcgccgtgagcagctgggcctacaggctggccgtgaggagctacgccggctgcagcaggaccacccccccccccaggtgcttcgccgaggccaggatggagcccgtgcccggcctggcctggctggccagcaccgtgaacctggagttccagcacgccagcccccagcacgccggcctgtacctgtgcgtggtgtacgtggacgaccacatccacgcctggggccacatgaccatcagcaccgccgcccagtacaggaacgccgtggtggagcagcacctgccccagaggcagcccgagcccgtggagcccaccaggccccacgtgagggcccccccccccgcccccagcgccaggggccccctgaggcgcaggcgctcctggcgggcggttaaaagccgggcctcggcgacgggccccacttacattcgcgtggcggacagcgagctgtacgcggactggagttcggacagcgagggggagcgcgacgggtccctgtggcaggaccctccggagagacccgactctccctccacaaatggatccggctttgagatcttatcaccaacggctccgtctgtatacccccatagcgaggggcgtaaatctcgccgcccgctcaccacctttggttcgggaagcccgggccgtcgtcactcccaggcctcctattcgtccgtcctctggtaa

SEQ ID NO: 77 - DNA, 324 bp, synthetic construct
  source 1..324: mol_type = other DNA
atgcgcaggcgctcctggcgggcggttaaaagccgggcctcggcgacgggccccacttacattcgcgtggcggacagcgagctgtacgcggactggagttcggacagcgagggggagcgcgacgggtccctgtggcaggaccctccggagagacccgactctccctccacaaatggatccggctttgagatcttatcaccaacggctccgtctgtatacccccatagcgaggggcgtaaatctcgccgcccgctcaccacctttggttcgggaagcccgggccgtcgtcactcccaggcctcctattcgtccgtcctctggtaa

SEQ ID NO: 78 - RNA, 318 nt, synthetic construct
  source 1..318: mol_type = other RNA
cggagaaggtcctggcgggccgtgaagtccagagcttctgctacaggacctacctatatcagagtggccgacagcgagctgtacgccgattggtccagcgacagcgaaggcgagcgggacggcagcctgtggcaggacccccccgagagacctgattctccaagcaccaacggctctggcttcgagatcctgagccctacagcccctagcgtctacccccactctgaaggaagaaagagcagacggcctctgaccaccttcggcagcggcagccctggcagaagacacagccaggccagctacagcagcgtgctgtgg

SEQ ID NO: 79 - RNA, 321 nt, synthetic construct
  source 1..321: mol_type = other RNA
atgcggagaaggtcctggcgggccgtgaagtccagagcttctgctacaggacctacctatatcagagtggccgacagcgagctgtacgccgattggtccagcgacagcgaaggcgagcgggacggcagcctgtggcaggacccccccgagagacctgattctccaagcaccaacggctctggcttcgagatcctgagccctacagcccctagcgtctacccccactctgaaggaagaaagagcagacggcctctgaccaccttcggcagcggcagccctggcagaagacacagccaggccagctacagcagcgtgctgtgg

SEQ ID NO: 80 - RNA, 637 nt, synthetic construct
  source 1..637: mol_type = other RNA
taatacgactcactataaggactcactatttgttttcgcgcccagttgcaaaaagtgtcgtcaccatgcggagaaggtcctggcgggccgtgaagtccagagcttctgctacaggacctacctatatcagagtggccgacagcgagctgtacgccgattggtccagcgacagcgaaggcgagcgggacggcagcctgtggcaggacccccccgagagacctgattctccaagcaccaacggctctggcttcgagatcctgagccctacagcccctagcgtctacccccactctgaaggaagaaagagcagacggcctctgaccaccttcggcagcggcagccctggcagaagacacagccaggccagctacagcagcgtgctgtgggagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 81 - RNA, 1251 nt, synthetic construct
  source 1..1251: mol_type = other RNA
atggccagaggcgccggactggttttcttcgtgggcgtttgggtggtgagctgtctggctgcagctccccggacaagctggaagcgggtgaccagcggcgaggacgtggtgctgctcccagctccagccggccctgaagagagaaccagagcccataagctgctgtgggccgccgaacccctggacgcctgtggccctctgagacctagctgggtggccctgtggcctcccagaagagtgctggagacagtggtggacgctgcttgcatgagggcccccgagcccctggccatcgcctacagcccccccttccctgcaggcgacgaaggcctgtactccgagctggcatggcgagacagagtggccgtggtgaacgagagcctggtgatctacggcgctctggaaaccgacagcggcctgtatacactgtctgtggtgggactgagcgatgaggctcgtcaggtggcctctgtggtcctggtggtcgagccagctcctgtgcccacccctacacctgacgactacgatgaggaagatgatgccggcgtgtctgagcggacccctgtgagcgtgccacctcctacccctcctcggagacctccagtcgccccacctacacaccctagagtgatccccgaagtgagccacgtgcgcggagtgaccgtgcacatggaaacccctgaggccatcctgttcgccccaggcgagaccttcggaacaaacgtgtccatccacgctattgcccacgacgacggcccttacgccatggacgtggtgtggatgcggttcgacgtgccctccagctgcgccgagatgagaatctacgaggcctgtctgtaccacccccagctgcctgagtgcctatctcctgctgacgccccgtgcgccgtgtccagctgggcctacagactggccgttagaagctacgccggctgctctcggaccacccctcctcctagatgctttgccgaggccaggatggaacccgtgcctggcctggcctggctggccagcaccgtgaatctggaatttcagcacgccagccctcaacacgctggcctgtatctgtgcgtggtgtacgtcgatgatcacatccacgcctggggacacatgaccatcagcacagccgcccagtaccggaacgccgtggtggagcagcatctgcctcagagacagcctgaacctgtggaacccacaagaccccacgtccgggcccctccgcccgccccaagcgccagaggccctctgaga

SEQ ID NO: 82 - RNA, 1567 nt, synthetic construct
  source 1..1567: mol_type = other RNA
taatacgactcactataaggactcactatttgttttcgcgcccagttgcaaaaagtgtcgtcaccatggccagaggcgccggactggttttcttcgtgggcgtttgggtggtgagctgtctggctgcagctccccggacaagctggaagcgggtgaccagcggcgaggacgtggtgctgctcccagctccagccggccctgaagagagaaccagagcccataagctgctgtgggccgccgaacccctggacgcctgtggccctctgagacctagctgggtggccctgtggcctcccagaagagtgctggagacagtggtggacgctgcttgcatgagggcccccgagcccctggccatcgcctacagcccccccttccctgcaggcgacgaaggcctgtactccgagctggcatggcgagacagagtggccgtggtgaacgagagcctggtgatctacggcgctctggaaaccgacagcggcctgtatacactgtctgtggtgggactgagcgatgaggctcgtcaggtggcctctgtggtcctggtggtcgagccagctcctgtgcccacccctacacctgacgactacgatgaggaagatgatgccggcgtgtctgagcggacccctgtgagcgtgccacctcctacccctcctcggagacctccagtcgccccacctacacaccctagagtgatccccgaagtgagccacgtgcgcggagtgaccgtgcacatggaaacccctgaggccatcctgttcgccccaggcgagaccttcggaacaaacgtgtccatccacgctattgcccacgacgacggcccttacgccatggacgtggtgtggatgcggttcgacgtgccctccagctgcgccgagatgagaatctacgaggcctgtctgtaccacccccagctgcctgagtgcctatctcctgctgacgccccgtgcgccgtgtccagctgggcctacagactggccgttagaagctacgccggctgctctcggaccacccctcctcctagatgctttgccgaggccaggatggaacccgtgcctggcctggcctggctggccagcaccgtgaatctggaatttcagcacgccagccctcaacacgctggcctgtatctgtgcgtggtgtacgtcgatgatcacatccacgcctggggacacatgaccatcagcacagccgcccagtaccggaacgccgtggtggagcagcatctgcctcagagacagcctgaacctgtggaacccacaagaccccacgtccgggcccctccgcccgccccaagcgccagaggccctctgagagagagctcgctttcttgctgtccaatttctattaaaggttcctttgttccctaagtccaactactaaactgggggatattatgaagggccttgagcatctggattctgcctaataaaaaacatttattttcattgctgcgtccaatcaacctctggattacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 83 - protein, 417 aa, synthetic construct
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLR

SEQ ID NO: 84 - protein, 1572 aa, Human alphaherpesvirus 2
AUGGCCAGGGGCGCCGGCCUGGUGUUCUUCGUGGGCGUGUGGGUGGUGAGCUGCCUGGCCGCCGCCCCCAGGACCAGCUGGAAGAGGGUGACCAGCGGCGAGGACGUGGUGCUGCUGCCCGCCCCCGCCGGCCCCGAGGAGAGGACCAGGGCCCACAAGCUGCUGUGGGCCGCCGAGCCCCUGGACGCCUGCGGCCCCCUGAGGCCCAGCUGGGUGGCCCUGUGGCCCCCCAGGAGGGUGCUGGAGACCGUGGUGGACGCCGCCUGCAUGAGGGCCCCCGAGCCCCUGGCCAUCGCCUACAGCCCCCCCUUCCCCGCCGGCGACGAGGGCCUGUACAGCGAGCUGGCCUGGAGGGACAGGGUGGCCGUGGUGAACGAGAGCCUGGUGAUCUACGGCGCCCUGGAGACCGACAGCGGCCUGUACACCCUGAGCGUGGUGGGCCUGAGCGACGAGGCCAGGCAGGUGGCCAGCGUGGUGCUGGUGGUGGAGCCCGCCCCCGUGCCCACCCCCACCCCCGACGACUACGACGAGGAGGACGACGCCGGCGUGAGCGAGAGGACCCCCGUGAGCGUGCCCCCCCCCACCCCCCCCAGGAGGCCCCCCGUGGCCCCCCCCACCCACCCCAGGGUGAUCCCCGAGGUGAGCCACGUGAGGGGCGUGACCGUGCACAUGGAGACCCCCGAGGCCAUCCUGUUCGCCCCCGGCGAGACCUUCGGCACCAACGUGAGCAUCCACGCCAUCGCCCACGACGACGGCCCCUACGCCAUGGACGUGGUGUGGAUGAGGUUCGACGUGCCCAGCAGCUGCGCCGAGAUGAGGAUCUACGAGGCCUGCCUGUACCACCCCCAGCUGCCCGAGUGCCUGAGCCCCGCCGACGCCCCCUGCGCCGUGAGCAGCUGGGCCUACAGGCUGGCCGUGAGGAGCUACGCCGGCUGCAGCAGGACCACCCCCCCCCCCAGGUGCUUCGCCGAGGCCAGGAUGGAGCCCGUGCCCGGCCUGGCCUGGCUGGCCAGCACCGUGAACCUGGAGUUCCAGCACGCCAGCCCCCAGCACGCCGGCCUGUACCUGUGCGUGGUGUACGUGGACGACCACAUCCACGCCUGGGGCCACAUGACCAUCAGCACCGCCGCCCAGUACAGGAACGCCGUGGUGGAGCAGCACCUGCCCCAGAGGCAGCCCGAGCCCGUGGAGCCCACCAGGCCCCACGUGAGGGCCCCCCCCCCCGCCCCCAGCGCCAGGGGCCCCCUGAGGCGCAGGCGCUCCUGGCGGGCGGUUAAAAGCCGGGCCUCGGCGACGGGCCCCACUUACAUUCGCGUGGCGGACAGCGAGCUGUACGCGGACUGGAGUUCGGACAGCGAGGGGGAGCGCGACGGGUCCCUGUGGCAGGACCCUCCGGAGAGACCCGACUCUCCCUCCACAAAUGGAUCCGGCUUUGAGAUCUUAUCACCAACGGCUCCGUCUGUAUACCCCCAUAGCGAGGGGCGUAAAUCUCGCCGCCCGCUCACCACCUUUGGUUCGGGAAGCCCGGGCCGUCGUCACUCCCAGGCCUCCUAUUCGUCCGUCCUCUGGUAA

SEQ ID NO: 85 - protein, 6 aa, synthetic construct
  MOD_RES 1: note = Amino acid residue is covalenty linked to C in position 13 in SEQ ID NO:55; note = other
MGBNFQ

SEQ ID NO: 86 - RNA, 291 nt, synthetic construct; identical to SEQ ID NO: 32
  source 1..291: mol_type = other RNA
  misc_feature 1..291: note = Codon-optimized UL11 of HSV-2 RNA sequence including UTRs and polyA, all uridine residues are 1-methyl-pseudouridine (m1f)

SEQ ID NO: 87 - RNA, 1119 nt, synthetic construct; identical to SEQ ID NO: 33
  source 1..1119: mol_type = other RNA
  misc_feature 1..1119: note = Codon-optimized UL16 of HSV-2 RNA sequence including UTRs and polyA, all uridine residues are 1-methyl-pseudouridine (m1f)

SEQ ID NO: 88 - RNA, 1599 nt, synthetic construct; identical to SEQ ID NO: 34
  source 1..1599: mol_type = other RNA
  misc_feature 1..1599: note = Codon-optimized UL21 of HSV-2 RNA sequence including UTRs and polyA, all uridine residues are 1-methyl-pseudouridine (m1f)